(19) United States
(12) Patent Application Publication
(10) Pub. No.: US 2026/0364813 A1
(43) Pub. Date: Nov. 19, 2026

(54) Behavior Control System

(71) Applicant: Softbank Group Corp., Tokyo (JP)

(72) Inventor: Masayoshi Son, Minato-ku,Tokyo (JP)

(21) Appl. No.: 19/475,766

(22) PCT Filed: Apr. 11, 2024
(86) PCT No.: PCT/JP2024/014728

§ 371 Date: Oct. 16, 2025

(30) Foreign Application Priority Data
Apr. 17, 2023 (JP) 2023-067475
Apr. 17, 2023 (JP) 2023-067476
Apr. 18, 2023 (JP) 2023-067886
Apr. 18, 2023 (JP) 2023-067888
Apr. 18, 2023 (JP) 2023-067890
Apr. 18, 2023 (JP) 2023-067892
Apr. 18, 2023 (JP) 2023-068148
Apr. 18, 2023 (JP) 2023067980
Apr. 19, 2023 (JP) 2023-068804
Apr. 19, 2023 (JP) 2023-068805
Apr. 19, 2023 (JP) 2023-068823
Apr. 19, 2023 (JP) 2023-068855
Apr. 20, 2023 (JP) 2023-069490
Apr. 20, 2023 (JP) 2023-069494
Apr. 20, 2023 (JP) 2023-069617
Apr. 20, 2023 (JP) 2023-069668
Apr. 20, 2023 (JP) 2023-069669

(51) Int. Cl. B25J 11/00; A01M 1/22; A61B 5/00; A61B 5/16; A61H 7/00; A63H 3/02; A63H 11/00; G06V 40/16; G06V 40/30; G09B 19/16; G10L 25/63

(52) U.S. Cl. CPC B25J 11/001; A61B 5/168; A61B 5/4836; A61H 7/007; A63H 3/02; A63H 11/00; A01M 1/226; A61H 2201/0149; A61H 2201/1659; A63H 2200/00; G06V 40/174; G06V 40/30; G09B 19/167; G10L 25/63

(57) ABSTRACT

A robot is caused to execute a behavior appropriate for a behavior of a user. A behavior control system includes an emotion determination unit that determines an emotion of a user or an emotion of a robot, and a behavior determination unit that generates a behavior content of a robot for a behavior of the user and the emotion of the user or the emotion of the robot and determines a behavior of the robot corresponding to the behavior content based on a sentence generation model having an interaction function of causing the user to interact with the robot. The behavior determination unit sets a behavior plan of the user based on an interaction between the user and the robot.

### TECHNICAL FIELD

The present disclosure relates to a behavior control system.

### BACKGROUND ART

Japanese Patent No. 6053847 discloses a technique for determining an appropriate behavior of a robot with respect to a state of a user. In the conventional technique of Japanese Patent No. 6053847, a reaction of a user when the robot executes a specific behavior is recognized, and in a case where a behavior of the robot with respect to the recognized reaction of the user cannot be determined, the behavior of the robot is updated by receiving information regarding the behavior suitable for the recognized state of the user from a server.

### SUMMARY OF INVENTION

### Technical Problem

However, in the related art, there is room for improvement in causing the robot to execute an appropriate behavior for the behavior of the user.

### Solution to Problem

According to a first aspect of the disclosure, a behavior control system is provided. The behavior control system includes a user state recognition unit that recognizes a state of a user including a behavior of the user, an emotion determination unit that determines at least one of an emotion of the user or an emotion of a robot, and a behavior determination unit that determines a behavior of the robot corresponding to the state of the user and the emotion of the user or a behavior of the robot corresponding to the state of the user and the emotion of the robot based on an interaction function of causing the user to interact with the robot, in which the robot is mounted on a conversational scheduler, and the behavior determination unit sets a behavior plan of the user based on an interaction between the user and the robot.

According to a second aspect of the disclosure, a behavior control system is provided. The behavior control system includes a user state recognition unit that recognizes a user state including a behavior of a user, an emotion determination unit that determines an emotion of the user or an emotion of a robot, and a behavior determination unit that determines a behavior of the robot corresponding to the user state and the emotion of the user or the emotion of the robot based on an interaction function that causes the user to interact with the robot, in which the robot is mounted on a ticket guidance system, and the behavior determination unit autonomously guides the user to an event while engaging in conversation with the user while observing a preference, a situation, and a reaction of the user.

According to a third aspect of the disclosure, a behavior control system is provided. The behavior control system includes a user state recognition unit that recognizes a user state including a behavior of a user, an emotion determination unit that determines an emotion of the user or an emotion of a robot, and a behavior determination unit that determines a behavior of the robot corresponding to the user state and the emotion of the user or the emotion of the robot based on a sentence generation model having an interaction function of causing the user to interact with the robot, in which the behavior determination unit determines a walking method to be proposed to the user based on an interaction result with the user by the sentence generation model.

According to a fourth aspect of the disclosure, a behavior control system is provided. The behavior control system includes a user state recognition unit that recognizes a user state including a behavior of a user, an emotion determination unit that determines an emotion of the user or an emotion of a robot, and a behavior determination unit that determines a behavior of the robot corresponding to the user state and the emotion of the user or the emotion of the robot based on a sentence generation model having an interaction function of causing the user to interact with the robot, in which the behavior determination unit presents the user with a condition of clothes or handling based on an interaction between the user and the robot.

According to a fifth aspect of the disclosure, a behavior control system is provided. The behavior control system includes a user state recognition unit that recognizes a user state including a behavior of a user, an emotion determination unit that determines an emotion of the user or an emotion of a robot, and a behavior determination unit that determines a behavior of the robot corresponding to the user state and the emotion of the user or the emotion of the robot based on a sentence generation model having an interaction function of causing the user to interact with the robot, in which the behavior determination unit determines processing content of an intercom based on the emotion of the user or the emotion of the robot.

The behavior determination unit may change at least one of an operation setting of the intercom, a response content to the user, or a response content to a visitor based on an emotion of the user or an emotion of the robot.

The robot may be mounted on a stuffed toy, or may be connected to a control target device mounted on the stuffed toy in a wireless or wired manner.

According to a sixth aspect of the disclosure, a behavior control system is provided. The behavior control system includes a user state recognition unit that recognizes a user state including a behavior of a user, an emotion determination unit that determines an emotion of the user or an emotion of a robot, and a behavior determination unit that determines a behavior of the robot corresponding to the user state and the emotion of the user or the emotion of the robot based on a sentence generation model having an interaction function of causing the user to interact with the robot, in which the behavior determination unit is capable of adjusting at least one of a temperature or elasticity of a mattress.

According to a seventh aspect of the disclosure, in the sixth aspect, the behavior determination unit is capable of individually adjusting a temperature for each part of the mattress.

According to an eighth aspect of the disclosure, in the sixth aspect, the behavior determination unit can individually adjust elasticity for each part of the mattress.

According to the eighth aspect of the disclosure, in the sixth aspect or the seventh aspect, the behavior determination unit may be configured to individually adjust elasticity for each part of the mattress.

According to a ninth aspect of the disclosure, in the sixth aspect, the temperature and the elasticity are maintained according to the user state.

In the ninth aspect of the disclosure, in any one aspect of the sixth to eighth aspects, the temperature and the elasticity may be maintained according to the user state.

According to a tenth aspect of the disclosure, in the sixth aspect, the robot is mounted on a stuffed toy, or is connected to a control target device mounted on the stuffed toy in a wireless or wired manner.

According to the tenth aspect of the disclosure, in any one aspect of the sixth to ninth aspects, the robot may be mounted on a stuffed toy, or may be connected to a control target device mounted on the stuffed toy in a wireless or wired manner.

According to an eleventh aspect of the disclosure, a behavior control system includes a user state recognition unit that recognizes a user state including a behavior of a user,
an emotion determination unit that determines an emotion of the user or an emotion of a robot, and
a behavior determination unit that determines a behavior of the robot corresponding to the user state and the emotion of the user or the emotion of the robot based on an interaction function of causing the user to interact with the robot, in which
the robot is mounted on a massage chair, and
the behavior determination unit changes at least one setting among kneading intensity, massage course, massage time, and massage part of the massage chair based on an emotion of the user or an emotion of the robot.

In addition, according to a twelfth aspect of the disclosure, the massage chair is connected to a control target device mounted on a stuffed toy in a wireless or wired manner.

According to a thirteenth aspect of the disclosure, a behavior control system is provided. The behavior control system includes a user state recognition unit that recognizes a user state including a behavior of a user, an emotion determination unit that determines an emotion of the user or an emotion of a robot, and a behavior determination unit that determines a behavior of the robot corresponding to the user state and the emotion of the user or the emotion of the robot based on a sentence generation model having an interaction function of causing the user to interact with the robot, in which the behavior determination unit determines processing content of a head-mounted display based on the emotion of the user or the emotion of the robot.

The behavior determination unit may change at least one of an operation time, image quality, or sound quality of the head-mounted display, and content to be reproduced on the head-mounted display based on the emotion of the user or the emotion of the robot.

In a case where a continuous use time of the head-mounted display by the user exceeds a set threshold value, the behavior determination unit may change at least one of the operation time, the image quality, and the sound quality of the head-mounted display, or the content to be reproduced on the head-mounted display to content more favorable to the health condition of the user as compared with before the threshold value was exceeded.

The robot may be mounted on a stuffed toy, or may be connected to a control target device mounted on the stuffed toy in a wireless or wired manner.

According to a fourteenth aspect of the disclosure, a behavior control system is provided. The behavior control system includes a user state recognition unit that recognizes a user state including a behavior of a user, an emotion determination unit that determines an emotion of the user or an emotion of a robot, and a behavior determination unit that determines a behavior of the robot corresponding to the user state and the emotion of the user or the emotion of the robot based on a sentence generation model having an interaction function of causing the user to interact with the robot, in which the behavior determination unit proposes a parameter to be set to an AI bath device to the user.

According to a fifteenth aspect of the disclosure, a behavior control system is provided. The behavior control system includes a user state recognition unit that recognizes a user state including a behavior of a user, an emotion determination unit that determines an emotion of the user or an emotion of a robot, and a behavior determination unit that determines a behavior of the robot corresponding to the user state and the emotion of the user or the emotion of the robot based on a sentence generation model having an interaction function of causing the user to interact with the robot, in which the behavior determination unit determines a behavior of uttering content corresponding to the user state of the user who uses fitness equipment.

According to a sixteenth aspect of the disclosure, in the fifteenth aspect, the user state recognition unit recognizes a conversation preference of the user, as the user state, and the behavior determination unit determines a behavior of uttering content according to the conversation preference of the user.

According to a seventeenth aspect of the disclosure, in the fifteenth aspect, the user state recognition unit recognizes a facial expression of the user as the user state, and the behavior determination unit determines a behavior of uttering content according to the facial expression of the user.

According to the seventeenth aspect of the disclosure, in the fifteenth aspect or the sixteenth aspect, the user state recognition unit may recognize a facial expression of the user as the user state, and the behavior determination unit determine a behavior of uttering content according to the facial expression of the user.

According to an eighteenth aspect of the disclosure, in the fifteenth aspect, the user state recognition unit recognizes a heart rate of the user as the user state, and the behavior determination unit determines a behavior of uttering content according to the heart rate of the user.

According to the eighteenth aspect of the disclosure, in any one aspect of the fifteenth to seventeenth aspects, the user state recognition unit may recognize a heart rate of the user as the user state, and the behavior determination unit may determine a behavior of uttering content according to the heart rate of the user.

According to a nineteenth aspect of the disclosure, in the fifteenth aspect, the user state recognition unit recognizes an exercise speed of the user as the user state, and the behavior determination unit determines a behavior of uttering content according to the exercise speed of the user.

According to the nineteenth aspect of the disclosure, in any one aspect of the fifteenth to eighteenth aspects, the user state recognition unit may recognize an exercise speed of the user as the user state, and the behavior determination unit may determine a behavior of uttering content according to the exercise speed of the user.

According to a twentieth aspect of the disclosure, in the fifteenth aspect, the robot is mounted on a stuffed toy, or is connected to a control target device mounted on the stuffed toy in a wireless or wired manner.

According to the twentieth aspect of the disclosure, in any one aspect of the fifteenth to nineteenth aspects, the robot may be mounted on a stuffed toy, or may be connected to a control target device mounted on the stuffed toy in a wireless or wired manner.

According to a twenty-first aspect of the disclosure, a behavior control system is provided. The behavior control system includes a user state recognition unit that recognizes a user state including a behavior of a user, an emotion determination unit that determines an emotion of the user or an emotion of a robot, and a behavior determination unit that determines a behavior of the robot corresponding to the user state and the emotion of the user or the emotion of the robot based on a sentence generation model having an interaction function of causing the user to interact with the robot, in which the behavior determination unit determines control on an actuator provided on a motorcycle as the behavior of the robot.

According to a twenty-second aspect of the disclosure, a behavior control system is provided. The behavior control system includes a user state recognition unit that recognizes a user state including a behavior of a user, an emotion determination unit that determines an emotion of the user or an emotion of a robot, and a behavior determination unit that determines a behavior of the robot corresponding to the user state and the emotion of the user or the emotion of the robot based on a sentence generation model having an interaction function of causing the user to interact with the robot, in which the behavior determination unit determines whether to generate an insect repelling ultrasonic wave around a fitting based on an interaction result with the user by the sentence generation model, or determines a response content to a visitor based on an interaction result with the user by the sentence generation model.

According to a twenty-third aspect of the disclosure, a behavior control system is provided. The behavior control system includes a user state recognition unit that recognizes a user state including a behavior of a user, an emotion determination unit that determines an emotion of the user or an emotion of a robot, and a behavior determination unit that determines a behavior of the robot corresponding to the user state and the emotion of the user or the emotion of the robot based on a sentence generation model having an interaction function of causing the user to interact with the robot, in which the behavior determination unit causes the robot to output a menu content based on the emotion of the user or the emotion of the robot.

Furthermore, according to a twenty-fourth aspect of the disclosure, the behavior control system includes an acquisition unit that acquires information regarding a temperature, humidity, or a season, and the behavior determination unit performs control to change content of a menu to be output by the robot, using the information regarding the temperature, the humidity, or the season.

Furthermore, according to a twenty-fifth aspect of the disclosure, the behavior control system includes an acquisition unit that acquires information regarding an ingredient, and the behavior determination unit performs control to change content of a menu to be output by the robot, using the information regarding the ingredient to be used.

Furthermore, according to a twenty-sixth aspect of the disclosure, the behavior control system includes an acquisition unit that acquires information regarding an inventory of a refrigerator, and the behavior determination unit performs control to change content of a menu to be output by the robot, using the information regarding the inventory of the refrigerator.

Furthermore, according to a twenty-seventh aspect of the disclosure, the behavior control system includes an acquisition unit that acquires information regarding a physical condition of a user, and the behavior determination unit performs control to change content of a menu to be output by the robot, using the information regarding the physical condition of the user.

Furthermore, according to a twenty-eighth aspect of the disclosure, the behavior determination unit performs control to change content of a menu to be output by the robot, using menu history data of a user.

Furthermore, according to a twenty-ninth aspect of the disclosure, the robot is mounted on a stuffed toy, or is connected to a control target device mounted on the stuffed toy in a wireless or wired manner.

According to a thirtieth aspect of the disclosure, a behavior control system is provided. The behavior control system includes a user state recognition unit that recognizes a user state including a behavior of a user, an emotion determination unit that determines an emotion of the user or an emotion of a robot, and a behavior determination unit that determines a behavior of the robot corresponding to the user state and the emotion of the user or the emotion of the robot based on a sentence generation model having an interaction function of causing the user to interact with the robot, in which the behavior determination unit determines, as the behavior, a response operation of the robot for the user whose numerical value of a body composition has been measured based on the sentence generation model.

According to a thirty-first aspect of the disclosure, a behavior control system is provided. The behavior control system includes a user state recognition unit that recognizes a user state including a behavior of a user, an emotion determination unit that determines an emotion of the user or an emotion of a robot, and a behavior determination unit that determines a behavior of the robot corresponding to the user state and the emotion of the user or the emotion of the robot based on a sentence generation model having an interaction function of causing the user to interact with the robot, in which the behavior determination unit determines, as a behavior of the robot, to identify preferred news of the user, to distribute the identified news, and to add a comment on the distributed news.

According to a thirty-second aspect of the disclosure, a behavior control system is provided. The behavior control system includes a user state recognition unit that recognizes a user state including a behavior of a user, an emotion determination unit that determines an emotion of the user or an emotion of a robot, and a behavior determination unit that determines a behavior of the robot corresponding to the user state and the emotion of the user or the emotion of the robot based on a sentence generation model having an interaction function of causing the user to interact with the robot, in which the behavior determination unit gives advice for improving a driving skill by engaging in conversation with a driver driving a vehicle based on at least one of the preference of the driver, the driving situation, or the reaction.

According to a thirty-third aspect of the disclosure, a behavior control system is provided. The behavior control system includes a user state recognition unit that recognizes a user state including a behavior of a user, an emotion determination unit that determines an emotion of the user or an emotion of a robot, and a behavior determination unit that determines a behavior of the robot corresponding to the user state and the emotion of the user or the emotion of the robot based on a sentence generation model having an interaction function of causing the user to interact with the robot, in which the behavior determination unit determines, as a behavior of the robot, providing a topic to a participant of a social gathering as the user, the topic facilitating interaction among the participants.

### BRIEF DESCRIPTION OF DRAWINGS

FIG. 1 schematically illustrates an example of a system 5 according to the present embodiment.

FIG. 2 schematically illustrates a functional configuration of a robot 100.

FIG. 3 schematically illustrates an example of an operation flow by the robot 100.

FIG. 4 schematically illustrates an example of a hardware configuration of a computer 1200.

FIG. 5 illustrates an emotion map 400 on which a plurality of emotions is mapped.

FIG. 6 illustrates an emotion map 900 on which a plurality of emotions is mapped.

FIG. 7(A) is an external view of a stuffed toy according to another embodiment, and FIG. 7(B) is an internal structural view of the stuffed toy.

FIG. 8 is a rear front view of a stuffed toy according to another embodiment.

### DESCRIPTION OF EMBODIMENTS

Hereinafter, embodiments of the disclosure will be described, but the following embodiments do not limit the invention according to the claims. In addition, not all combinations of features described in the embodiments are essential to the solution of the disclosure.

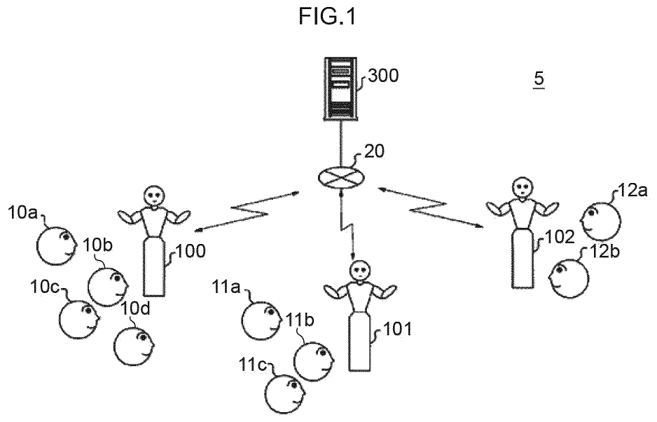

FIG. 1 schematically illustrates an example of a system 5 according to the present embodiment. The system 5 includes a robot 100, a robot 101, a robot 102, and a server 300. A user 10a, a user 10b, a user 10c, and a user 10d are users of the robot 100. A user 11a, a user 11b, and a user 11c are users of the robot 101. A user 12a and a user 12b are users of the robot 102. Note that, in the description of the present embodiment, the user 10a, the user 10b, the user 10c, and the user 10d may be collectively referred to as a user 10. Furthermore, the user 11a, the user 11b, and the user 11c may be collectively referred to as a user 11. Furthermore, the user 12a and the user 12b may be collectively referred to as a user 12. The robot 101 and the robot 102 have substantially the same functions as those of the robot 100. Therefore, the system 5 will be described focusing on the function of the robot 100.

The robot 100 has a conversation with the user 10 and provides an image to the user 10. At this time, the robot 100 provides a conversation with the user 10, an image to the user 10, and the like in cooperation with the server 300 and the like that can communicate via the communication network 20. For example, the robot 100 not only learns an appropriate conversation by itself, but also performs learning such that a conversation with the user 10 can be advanced more appropriately in cooperation with the server 300. Furthermore, the robot 100 causes the server 300 to record the captured image data and the like of the user 10, if necessary, requests the server 300 to provide the image data and the like, and provides image data and the like to the user 10.

Furthermore, the robot 100 has an emotion value indicating the type of its own emotion. For example, the robot 100 has emotion values representing the intensity of each emotion of “HAPPINESS”, “ANGER”, “GRIEF”, “ENJOYMENT”, “COMFORT”, “DISCOMFORT”, “RELIEF”, “ANXIETY”, “SADNESS”, “EXCITEMENT”, “CONCERN”, “SENSE OF RELIEF”, “SENSE OF FULFILLMENT”, “SENSE OF EMPTINESS”, and “ORDINARY”. For example, when the robot 100 has a conversation with the user 10 in a state where the emotion value of excitement is high, the robot outputs voice at a fast speed. In this regard, the robot 100 can express its own emotion through behavior.

Furthermore, the robot 100 may be configured to determine the behavior of the robot 100 corresponding to the emotion of the user 10 by matching a sentence generation model using artificial intelligence (AI) with an emotion engine. Specifically, the robot 100 may be configured to recognize the behavior of the user 10, determine an emotion of the user 10 for the behavior of the user, and determine the behavior of the robot 100 corresponding to the determined emotion.

More specifically, in a case where it is recognized of the behavior of the user 10, the robot 100 automatically generates the behavior content to be taken by the robot 100 with respect to the behavior of the user 10 using the preset sentence generation model. The sentence generation model may be interpreted as an algorithm and an operation for automatic interaction processing with characters. Since the sentence generation model is known as disclosed in, for example, Japanese Patent Application Laid-Open (JP-A) No. 2018-081444 and chatGPT (Internet search <URL: https://openai.com/blog/chatgpt>), a detailed description thereof will be omitted. Such a sentence generation model is configured using a large language model (LLM).

As described above, in the present embodiment, it is possible to reflect the emotions of the user 10 and the robot 100 and various linguistic information in the behavior of the robot 100 by combining the large language model and the emotion engine. That is, according to the present embodiment, a synergistic effect can be obtained by combining the sentence generation model and the emotion engine.

Furthermore, the robot 100 has a function of recognizing the behavior of the user 10. The robot 100 recognizes the behavior of the user 10 by analyzing the face image of the user 10 acquired by a camera function and the voice of the user 10 acquired by a microphone function. The robot 100 determines a behavior to be executed by the robot 100 based on the recognized behavior of the user 10 or the like.

The robot 100 stores a rule defining a behavior to be executed by the robot 100 based on the emotion of the user 10, the emotion of the robot 100, and the behavior of the user 10, and performs various behaviors according to the rule.

Specifically, the robot 100 has a reaction rule for determining the behavior of the robot 100 based on the emotion of the user 10, the emotion of the robot 100, and the behavior of the user 10. In the reaction rule, for example, in a case where the behavior of the user 10 is “LAUGHING”, the behavior of “LAUGHING” is defined as the behavior of the robot 100. Furthermore, in the reaction rule, in a case where the behavior of the user 10 is “GETTING ANGRY”, the behavior of “APOLOGIZING” is defined as the behavior of the robot 100. Furthermore, in the reaction rule, in a case where the behavior of the user 10 is “ASKING A QUESTION”, the behavior of “ANSWERING” is defined as the behavior of the robot 100. In the reaction rule, in a case where the behavior of the user 10 is “BEING SAD”, the behavior of “SPEAKING TO” is defined as the behavior of the robot 100.

In a case where it is recognized that the behavior of the user 10 is “GETTING ANGRY” based on the reaction rule, the robot 100 selects a behavior “APOLOGIZING” defined in the reaction rule as the behavior to be executed by the robot 100. For example, in a case where the behavior of “APOLOGIZING” is selected, the robot 100 performs an “APOLOGIZING” operation and outputs voice representing words of “APOLOGIZING”.

In addition, in a case where conditions that the emotion of the robot 100 is “ORDINARY” (that is, “HAPPINESS”=0, “ANGER”=0, “GRIEF”=0, and “ENJOYMENT”=0) and the state of the user 10 is “ALONE AND SEEMINGLY LONELY” are satisfied, it is defined that the change content of the emotion that the emotion of the robot 100 is “CONCERNED” and the behavior of “SPEAKING TO” can be executed.

In a case where the current emotion of the robot 100 is “ORDINARY” and the user 10 is recognized as being in a lonely state alone based on the reaction rule, the robot 100 increases the emotion value of “GRIEF” of the robot 100. In addition, the robot 100 selects a behavior of “SPEAKING TO” defined in the reaction rule as the behavior to be executed on the user 10. For example, in a case where the behavior of “SPEAKING TO” is selected, the robot 100 converts a word “WHAT'S WRONG?” which expresses concern into a concerned-sounding voice and outputs the voice.

Furthermore, the robot 100 transmits user reaction information indicating that a positive reaction has been obtained from the user 10 by this behavior to the server 300. The user reaction information includes, for example, a user behavior of “GETTING ANGRY”, a behavior of the robot 100 of “APOLOGIZING”, a positive reaction of the user 10, and an attribute of the user 10.

The server 300 stores the user reaction information received from the robot 100. The server 300 receives and stores the user reaction information not only from the robot 100 but also from each of the robot 101 and the robot 102. Then, the server 300 analyzes the user reaction information from the robot 100, the robot 101, and the robot 102, and updates the reaction rule.

The robot 100 receives the updated reaction rule from the server 300 by inquiring the server 300 about the updated reaction rule. The robot 100 incorporates the updated reaction rule into the reaction rule stored in the robot 100. As a result, the robot 100 can incorporate the reaction rule acquired by the robot 101, the robot 102, or the like into its own reaction rule.

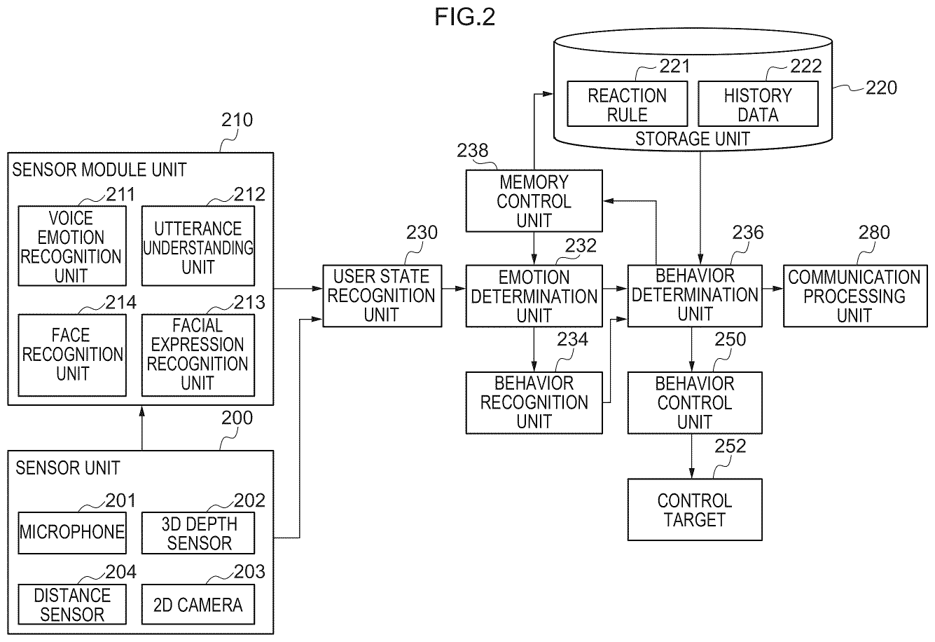

FIG. 2 schematically illustrates a functional configuration of the robot 100. The robot 100 includes a sensor unit 200, a sensor module unit 210, a storage unit 220, a user state recognition unit 230, an emotion determination unit 232, a behavior recognition unit 234, a behavior determination unit 236, a memory control unit 238, a behavior control unit 250, a control target 252, and a communication processing unit 280.

The control target 252 includes a display apparatus, a speaker, an LED of an eye portion, and a motor that drives an arm, a hand, a foot, and the like. The posture and the movement of the robot 100 are controlled by controlling motors of arms, hands, feet, and the like. Some of the emotions of the robot 100 can be expressed by controlling these motors. Furthermore, the facial expression of the robot 100 can be expressed by controlling the light emission state of the LED of the eye portion of the robot 100. The posture, movement, and facial expression of the robot 100 are examples of the attitude of the robot 100.

The sensor unit 200 includes a microphone 201, a 3D depth sensor 202, a 2D camera 203, and a distance sensor 204. The microphone 201 continuously detects a voice and outputs voice data. The microphone 201 may be provided on the head portion of the robot 100 and may have a function of performing binaural recording. The 3D depth sensor 202 detects the contour of the object by continuously radiating the infrared pattern and analyzing the infrared pattern from the infrared image continuously captured by the infrared camera. The 2D camera 203 is an example of an image sensor. The 2D camera 203 captures an image with visible light and generates image information of visible light. The distance sensor 204 detects a distance to an object by emitting, for example, a laser, an ultrasonic wave, or the like. The sensor unit 200 may further include a clock, a gyro sensor, a touch sensor, a sensor for motor feedback, and the like.

Note that, among the components of the robot 100 illustrated in FIG. 2, the components other than the control target 252 and the sensor unit 200 are examples of the components included in the behavior control system included in the robot 100. The behavior control system of the robot 100 controls the control target 252.

The storage unit 220 includes a reaction rule 221 and history data 222. The history data 222 includes a history of past emotion values and behaviors of the user 10. The history of the emotion values and the behaviors is recorded for each user 10 by associating it with identification information of the user 10, for example. At least a part of the storage unit 220 is implemented by a storage medium such as a memory. A person DB that stores a face image of the user 10, attribute information of the user 10, and the like may be included. Note that, among the components of the robot 100 illustrated in FIG. 2, the functions of the components other than the control target 252, the sensor unit 200, and the storage unit 220 can be realized by the CPU operating based on a program. For example, the functions of these components can be implemented as the operation of the CPU by basic software (OS) and a program operating on the OS.

The sensor module unit 210 includes a voice emotion recognition unit 211, an utterance understanding unit 212, a facial expression recognition unit 213, and a face recognition unit 214. Information detected by the sensor unit 200 is input to the sensor module unit 210. The sensor module unit 210 analyzes information detected by the sensor unit 200 and outputs an analysis result to the user state recognition unit 230.

The voice emotion recognition unit 211 of the sensor module unit 210 analyzes the voice of the user 10 detected by the microphone 201 to recognize the emotion of the user 10. For example, the voice emotion recognition unit 211 extracts a feature amount such as a frequency component of voice and recognizes the emotion of the user 10 based on the extracted feature amount. The utterance understanding unit 212 analyzes the voice of the user 10 detected by the microphone 201 and outputs character information representing the utterance content of the user 10.

The facial expression recognition unit 213 recognizes the facial expression of the user 10 and the emotion of the user 10 from the image of the user 10 captured by the 2D camera 203. For example, the facial expression recognition unit 213 recognizes the facial expression and the emotion of the user 10 based on the shapes, positional relationships, and the like of the eyes and the mouth.

The face recognition unit 214 recognizes the face of the user 10. The face recognition unit 214 recognizes the user 10 by matching a face image stored in a person DB (not illustrated) with a face image of the user 10 captured by the 2D camera 203.

The user state recognition unit 230 recognizes the state of the user 10 based on the information analyzed by the sensor module unit 210. For example, processing mainly related to perception is performed using the analysis result of the sensor module unit 210. For example, perception information such as “PAPA IS ALONE.” and “THERE IS A 90% PROBABILITY THAT PAPA IS NOT SMILING.” is generated. Processing of understanding the meaning of the generated perception information is performed. For example, semantic information such as “PAPA IS ALONE AND SEEMS LONELY.” is generated.

The emotion determination unit 232 determines an emotion value indicating the emotion of the user 10 based on the information analyzed by the sensor module unit 210 and the state of the user 10 recognized by the user state recognition unit 230. For example, the information analyzed by the sensor module unit 210 and the recognized state of the user 10 are input to a neural network learned in advance, and an emotion value indicating the emotion of the user 10 is acquired.

Here, the emotion value indicating the emotion of the user 10 is a value indicating whether the emotion of the user is positive or negative. For example, in a case where the emotion of the user is a bright emotion accompanied with a pleasant feeling or a sense of relief, such as “HAPPINESS”, “ENJOYMENT”, “COMFORT”, “RELIEF”, “EXCITEMENT”, “SENSE OF RELIEF”, or “SENSE OF FULFILLMENT”, a positive value is indicated, and the value becomes higher as the emotion is brighter. If the emotion of the user is an emotion that makes the user feel unpleasant, such as “ANGER”, “GRIEF”, “DISCOMFORT”, “ANXIETY”, “SADNESS”, “CONCERN”, and “SENSE OF EMPTINESS”, a negative value is indicated, and the absolute value of the negative value increases as the user feels unpleasant. In a case where the emotion of the user is not any of the above (“ORDINARY”), a value of 0 is indicated.

In addition, the emotion determination unit 232 determines an emotion value indicating the emotion of the robot 100 based on the information analyzed by the sensor module unit 210 and the state of the user 10 recognized by the user state recognition unit 230.

The emotion value of the robot 100 includes the emotion value for each of the plurality of emotion classifications, and is, for example, a value (0 to 5) indicating the intensity of each of “HAPPINESS”, “ANGER”, “GRIEF”, and “ENJOYMENT”.

Specifically, the emotion determination unit 232 determines an emotion value indicating the emotion of the robot 100 according to a rule for updating the emotion value of the robot 100 defined in association with the information analyzed by the sensor module unit 210 and the state of the user 10 recognized by the user state recognition unit 230.

For example, in a case where the user state recognition unit 230 recognizes that the user 10 looks lonely, the emotion determination unit 232 increases the emotion value of “GRIEF” of the robot 100. Furthermore, in a case where the user state recognition unit 230 recognizes that the user 10 has a smiling face, the emotion value of “HAPPINESS” of the robot 100 is increased.

The emotion determination unit 232 may determine the emotion value indicating the emotion of the robot 100 in further consideration of the state of the robot 100. For example, in a case where the remaining battery level of the robot 100 is low, a case where the surrounding environment of the robot 100 is dark, or the like, the emotion value of “GRIEF” of the robot 100 may be increased. Furthermore, in the case of the user 10 who continues to speak to the robot despite the remaining battery level being low, the emotion value of “ANGER” may be increased.

The behavior recognition unit 234 recognizes a behavior of the user 10 based on the information analyzed by the sensor module unit 210 and the state of the user 10 recognized by the user state recognition unit 230. For example, the information analyzed by the sensor module unit 210 and the recognized state of the user 10 are input to a neural network learned in advance, probabilities of a plurality of predetermined behavior classifications (for example, “LAUGHING”, “GETTING ANGRY”, “ASKING A QUESTION”, and “BEING SAD”) are acquired, and the behavior classification having the highest probability is recognized as the behavior of the user 10.

As described above, in the present embodiment, the robot 100 acquires the utterance content of the user 10 after identifying the user 10; however, in acquiring and using the utterance content, the behavior control system of the robot 100 according to the present embodiment considers protection of personal information and privacy of the user 10 in addition to acquiring necessary consent according to laws and regulations from the user 10.

The behavior determination unit 236 determines a behavior corresponding to the behavior of the user 10 recognized by the behavior recognition unit 234 based on the current emotion value of the user 10 determined by the emotion determination unit 232, the history data 222 of the past emotion values determined by the emotion determination unit 232 before the current emotion value of the user 10 is determined, and the emotion value of the robot 100. In the present embodiment, a case where the behavior determination unit 236 uses one most recent emotion value included in the history data 222 as the past emotion value of the user 10 will be described; however, the disclosed technique is not limited to this aspect. For example, the behavior determination unit 236 may use a plurality of most recent emotion values as the past emotion values of the user 10, or may use emotion values that are earlier by a unit period such as one day before. Furthermore, the behavior determination unit 236 may determine a behavior corresponding to the behavior of the user 10 in further consideration of the history of the past emotion values of the robot 100 in addition to the current emotion value of the robot 100. The behavior determined by the behavior determination unit 236 includes a gesture performed by the robot 100 or utterance content of the robot 100.

The behavior determination unit 236 according to the present embodiment determines the behavior of the robot 100 based on a combination of the past emotion value and the current emotion value of the user 10, the emotion value of the robot 100, the behavior of the user 10, and the reaction rule 221 as the behavior corresponding to the behavior of the user 10. For example, in a case where the past emotion value of the user 10 is a positive value and the current emotion value is a negative value, the behavior determination unit 236 determines a behavior for positively changing the emotion value of the user 10 as a behavior corresponding to the behavior of the user 10.

In the reaction rule 221, the behavior of the robot 100 according to the combination of the past emotion value and the current emotion value of the user 10, the emotion value of the robot 100, and the behavior of the user 10 is determined. For example, in a case where the past emotion value of the user 10 is a positive value, the current emotion value is a negative value, and the behavior of the user 10 is being sad, a combination of a gesture and utterance content to encourage the user 10 with a question, while incorporating the gesture, is determined as the behavior of the robot 100.

For example, in the reaction rule 221, the behavior of the robot 100 is determined for all combinations of the pattern of the emotion value of the robot 100 (1,296 patterns, which is obtained as the fourth power of the six values from “0” to “5” assigned respectively to “HAPPINESS”, “ANGER”, “GRIEF”, and “ENJOYMENT”), the pattern of the combination of the past emotion value and the current emotion value of the user 10, and the behavior pattern of the user 10. That is, for each pattern of the emotion value of the robot 100, the behavior of the robot 100 according to the behavior pattern of the user 10 is determined for each of a plurality of combinations such that the combination of the past emotion value and the current emotion value of the user 10 is a negative value and a negative value, a negative value and a positive value, a positive value and a negative value, a positive value and a positive value, a negative value and an ordinary, and an ordinary and an ordinary. Note that the behavior determination unit 236 may transition to the operation mode of determining the behavior of the robot 100 using the history data 222, for example, in a case where the user 10 has made an utterance that intends a conversation continued from a past topic such as “I WANT TO TALK ABOUT THAT TOPIC WE DISCUSSED BEFORE”.

Note that, in the reaction rule 221, for each of the patterns (1296 patterns) of the emotion value of the robot 100, at most one gesture and at most one statement content may be respectively defined as a behavior of the robot 100. Alternatively, in the reaction rule 221, at least one of a gesture or a statement content may be defined as a behavior of the robot 100 for each of the groups of the patterns of the emotion values of the robot 100.

The intensity of each gesture included in the behavior of the robot 100 defined in the reaction rule 221 is defined in advance. The intensity of the utterance content is defined in advance in each utterance content included in the behavior of the robot 100 defined in the reaction rule 221.

The memory control unit 238 determines whether to store data including the behavior of the user 10 in the history data 222 based on the intensity of the behavior determined in advance for the behavior determined by the behavior determination unit 236 and the emotion value of the robot 100 determined by the emotion determination unit 232.

Specifically, in a case where the sum of the emotion values for each of a plurality of emotion classifications of the robot 100 and a total value of the intensities, which is the sum of a predetermined intensity for a gesture included in the behavior determined by the behavior determination unit 236 and a predetermined intensity for utterance content included in the behavior determined by the behavior determination unit 236 is equal to or greater than a threshold value, it is determined to store data including the behavior of the user 10 in the history data 222.

In a case where it is determined that the data including the behavior of the user 10 is stored in the history data 222, the memory control unit 238 stores, in the history data 222, the behavior determined by the behavior determination unit 236, the information (for example, any peripheral information such as data such as voice, an image, and a smell of the place) analyzed by the sensor module unit 210 from the current point of time to a certain period of time before, and the state (for example, the facial expression, emotion, and the like of the user 10) of the user 10 recognized by the user state recognition unit 230.

The behavior control unit 250 controls the control target 252 based on the behavior determined by the behavior determination unit 236. For example, in a case where the behavior determination unit 236 has determined a behavior including an utterance, the behavior control unit 250 causes a speaker included in the control target 252 to output voice. At this time, the behavior control unit 250 may determine the utterance speed of the voice based on the emotion value of the robot 100. For example, the behavior control unit 250 determines a higher utterance speed as the emotion value of the robot 100 is larger. In this regard, the behavior control unit 250 determines an execution mode of the behavior determined by the behavior determination unit 236 based on the emotion value determined by the emotion determination unit 232.

The behavior control unit 250 may recognize a change in emotion of the user 10 with respect to execution of the behavior determined by the behavior determination unit 236. For example, the change in emotion may be recognized based on the voice or facial expression of the user 10. In addition, a change in emotion of the user 10 may be recognized based on detection of an impact by the touch sensor included in the sensor unit 200. In a case where an impact is detected by the touch sensor included in the sensor unit 200, it may be recognized that the emotion of the user 10 is worsened, or in a case where it is determined that the reaction of the user 10 is a laugh or happy from the detection result of the touch sensor included in the sensor unit 200, it may be recognized that the emotion of the user 10 is improved. Information indicating the reaction of the user 10 is output to the communication processing unit 280.

Furthermore, after the behavior control unit 250 executes the behavior determined by the behavior determination unit 236 in the execution mode determined according to the emotion of the robot 100, the emotion determination unit 232 further changes the emotion value of the robot 100 based on the reaction of the user to the execution of the behavior. Specifically, the emotion determination unit 232 increases the emotion value of “HAPPINESS” of the robot 100 in a case where the reaction of the user to the behavior determined by the behavior determination unit 236 performed on the user in the execution mode determined by the behavior control unit 250 is not poor, and the emotion determination unit 232 increases the emotion value of “GRIEF” of the robot 100 in a case where the reaction of the user to the behavior determined by the behavior determination unit 236 performed on the user in the execution mode determined by the behavior control unit 250 is poor.

Furthermore, the behavior control unit 250 expresses the emotion of the robot 100 based on the determined emotion value of the robot 100. For example, in a case where the emotion value of “HAPPINESS” of the robot 100 is increased, the behavior control unit 250 controls the control target 252 to cause the robot 100 to make a movement of joy. Furthermore, in a case where the emotion value of “GRIEF” of the robot 100 is increased, the behavior control unit 250 controls the control target 252 such that the posture of the robot 100 becomes a slumped posture.

The communication processing unit 280 is responsible for communication with the server 300. As described above, the communication processing unit 280 transmits the user reaction information to the server 300. Furthermore, the communication processing unit 280 receives the updated reaction rule from the server 300. When receiving the updated reaction rule from the server 300, the communication processing unit 280 updates the reaction rule 221.

The server 300 performs communication between the robot 100, the robot 101, and the robot 102 and the server 300, receives the user reaction information transmitted from the robot 100, and updates the reaction rule based on the reaction rule including the behavior for which a positive reaction has been obtained.

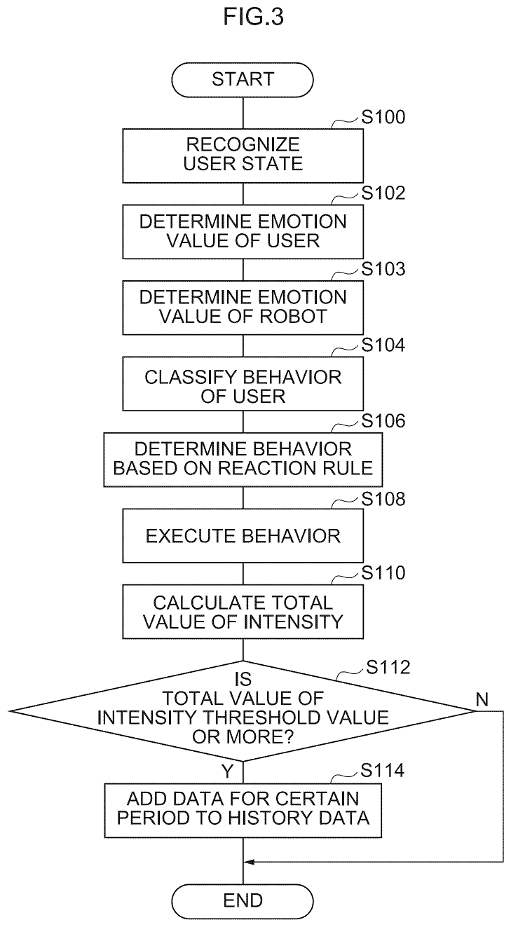

FIG. 3 schematically illustrates an example of an operation flow related to an operation of determining a behavior in the robot 100. The operation flow illustrated in FIG. 3 is repeatedly executed. At this time, it is assumed that information analyzed by the sensor module unit 210 is input. Note that “S” in the operation flow represents a step to be executed.

First, in step S100, the user state recognition unit 230 recognizes the state of the user 10 based on the information analyzed by the sensor module unit 210.

In step S102, the emotion determination unit 232 determines an emotion value indicating the emotion of the user 10 based on the information analyzed by the sensor module unit 210 and the state of the user 10 recognized by the user state recognition unit 230.

In step S103, the emotion determination unit 232 determines an emotion value indicating the emotion of the robot 100 based on the information analyzed by the sensor module unit 210 and the state of the user 10 recognized by the user state recognition unit 230. The emotion determination unit 232 adds the determined emotion value of the user 10 to the history data 222.

In step S104, the behavior recognition unit 234 recognizes a behavior classification of the user 10 based on the information analyzed by the sensor module unit 210 and the state of the user 10 recognized by the user state recognition unit 230.

In step S106, the behavior determination unit 236 determines the behavior of the robot 100 based on the combination of the current emotion value of the user 10 determined in step S102 and the past emotion value included in the history data 222, the emotion value of the robot 100, the behavior of the user 10 recognized by the behavior recognition unit 234, and the reaction rule 221.

In step S108, the behavior control unit 250 controls the control target 252 based on the behavior determined by the behavior determination unit 236.

In step S110, the memory control unit 238 calculates a total value of the intensities based on the intensity of the behavior predetermined for the behavior determined by the behavior determination unit 236 and the emotion value of the robot 100 determined by the emotion determination unit 232.

In step S112, the memory control unit 238 determines whether the total value of the intensities is equal to or greater than the threshold value. In a case where the total value of the intensities is less than the threshold value, the data including the behavior of the user 10 is not stored in the history data 222, and the processing ends. On the other hand, in a case where the total value of the intensities is equal to or greater than the threshold value, the processing proceeds to step S114.

In step S114, the behavior determined by the behavior determination unit 236, the information analyzed by the sensor module unit 210 from the current time point to a certain period before, and the state of the user 10 recognized by the user state recognition unit 230 are stored in the history data 222.

As described above, according to the robot 100, the emotion value indicating the emotion of the robot 100 is determined based on the user state, and whether to store data including the behavior of the user 10 in the history data 222 is determined based on the emotion value of the robot 100. As a result, the capacity of the history data 222 that stores data including the behavior of the user 10 can be suppressed. Then, for example, when the robot 100 determines that the user state 10 years later is the same as the state 10 years ago, the robot 100 can present to the user 10 the state of the user 10 at that time 10 years ago (for example, the facial expression and emotion of the user 10), and furthermore all peripheral information such as data of voices, images, and smells at that place, by reading out the history data 222 from 10 years ago.

Furthermore, according to the robot 100, it is possible to cause the robot 100 to execute an appropriate behavior with respect to the behavior of the user 10. Conventionally, a behavior of a user is classified to determine a behavior including a facial expression or an appearance of a robot. On the other hand, the robot 100 determines the current emotion value of the user 10, and executes a behavior on the user 10 based on the past emotion value and the current emotion value. Therefore, for example, in a case where the user 10 who was fine yesterday is depressed today, the robot 100 can make an utterance such as “YOU WERE FINE YESTERDAY. WHAT'S WRONG WITH YOU TODAY?”. Furthermore, the robot 100 can also make an utterance with a gesture. Furthermore, for example, in a case where the user 10 who was depressed yesterday is fine today, the robot 100 can make an utterance such as, “YOU WERE DEPRESSED YESTERDAY, BUT YOU LOOK FINE TODAY?”. Furthermore, for example, in a case where the user 10 who was fine yesterday is better today than yesterday, the robot 100 can make an utterance such as “YOU LOOK BETTER TODAY THAN YESTERDAY. DID SOMETHING BETTER HAPPEN THAN YESTERDAY?”. Furthermore, for example, the robot 100 can make an utterance such as “YOUR MOOD HAS BEEN STABLE RECENTLY, AND THAT'S NICE.” to the user 10 whose emotion value is 0 or more and whose state in which the fluctuation range of the emotion value is within a certain range continues.

Furthermore, for example, in a case where the robot 100 asks a question of “DID YOU FINISH THE HOMEWORK YOU MENTIONED YESTERDAY?” to the user 10 and an answer of “I DID IT” is obtained from the user 10, the robot can make an affirmative utterance such as “GOOD JOB!” and make an affirmative gesture such as applause or thumbs-up. Furthermore, for example, when the user 10 utters such as “THE PRESENTATION WE DISCUSSED THE DAY BEFORE YESTERDAY WAS SUCCESSFUL”, the robot 100 can make an affirmative utterance such as “WELL DONE!” and also make the above affirmative gesture. In this regard, the robot 100 performs a behavior based on the history of the state of the user 10, whereby the user 10 can be expected to feel a sense of closeness to the robot 100.

In the above embodiment, the case where the robot 100 recognizes the user 10 using the face image of the user 10 has been described; however, the disclosed technique is not limited to this aspect. For example, the robot 100 may recognize the user 10 using a voice uttered by the user 10, an e-mail address of the user 10, an ID of an SNS of the user 10, an ID card incorporating a wireless IC tag possessed by the user 10, or the like.

The robot 100 is an example of an electronic device including a behavior control system. The application target of the behavior control system is not limited to the robot 100, and the behavior control system can be applied to various electronic devices. Furthermore, the function of the server 300 may be implemented by one or more computers. At least some functions of the server 300 may be implemented by a virtual machine. Furthermore, at least a part of the functions of the server 300 may be implemented in a cloud.

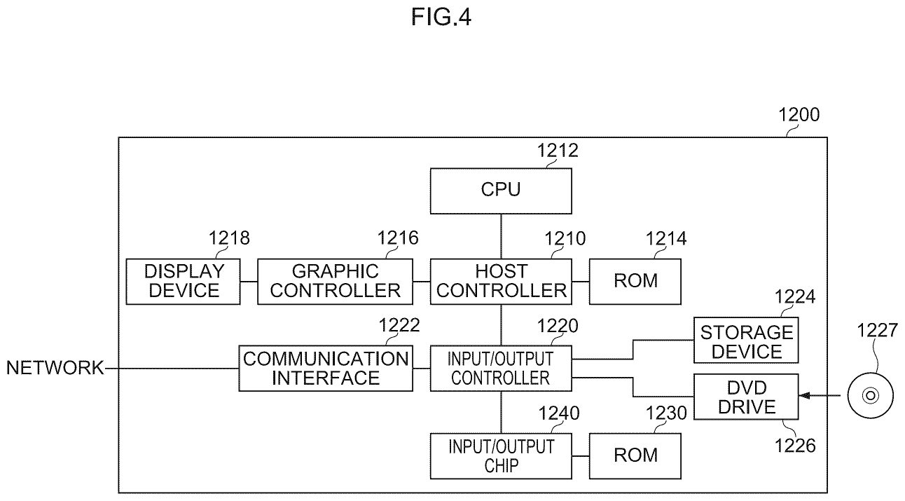

FIG. 4 schematically illustrates an example of a hardware configuration of a computer 1200 that functions as the robot 100 and the server 300. The program installed in the computer 1200 can cause the computer 1200 to function as one or more “units” of the device according to the present embodiment, or cause the computer 1200 to execute an operation associated with the device according to the present embodiment or one or more “units” thereof, and/or cause the computer 1200 to execute a process according to the present embodiment or a stage of the process. Such programs may be executed by the CPU 1212 to cause the computer 1200 to perform certain operations associated with some or all of the blocks in the flowcharts and block diagrams described in the present specification.

The computer 1200 according to the present embodiment includes a CPU 1212, a RAM 1214, and a graphic controller 1216, which are mutually connected by a host controller 1210. The computer 1200 also includes input/output units such as a communication interface 1222, a storage device 1224, a DVD drive 1226, and an IC card drive, which are connected to the host controller 1210 via the input/output controller 1220. The DVD drive 1226 may be a DVD-ROM drive, a DVD-RAM drive, or the like. The storage device 1224 may be a hard disk drive, a solid state drive, or the like. The computer 1200 also includes a ROM 1230 and legacy input/output units such as a keyboard, which are connected to an input/output controller 1220 via an input/output chip 1240.

The CPU 1212 operates according to programs stored in the ROM 1230 and the RAM 1214, thereby controlling each unit. The graphic controller 1216 acquires image data generated by the CPU 1212 in a frame buffer or the like provided in the RAM 1214 or itself, and causes the image data to be displayed on the display device 1218.

The communication interface 1222 communicates with other electronic devices via a network. The storage device 1224 stores programs and data used by the CPU 1212 in the computer 1200. The DVD drive 1226 reads out a program or data from a DVD-ROM 1227 or the like and provides the program or data to the storage device 1224. The IC card drive reads out the program and data from the IC card and/or writes the program and data to the IC card.

The ROM 1230 stores therein a boot program executed by the computer 1200 at the time of activation and/or a program depending on hardware of the computer 1200. The input/output chip 1240 may also connect various input/output units to the input/output controller 1220 via a USB port, a parallel port, a serial port, a keyboard port, a mouse port, or the like.

The program is provided by a computer-readable storage medium such as the DVD-ROM 1227 or an IC card. The program is read from a computer-readable storage medium, installed in the storage device 1224, the RAM 1214, or the ROM 1230, which is also an example of a computer-readable storage medium, and executed by the CPU 1212. The information processing described in these programs is read by the computer 1200 and provides cooperation between the programs and the various types of hardware resources. The device or method may be configured by implementing operation or processing of information according to use of the computer 1200.

For example, in a case where communication is performed between the computer 1200 and an external device, the CPU 1212 may execute a communication program loaded in the RAM 1214 and instruct the communication interface 1222 to perform communication processing based on processing described in the communication program. Under the control of the CPU 1212, the communication interface 1222 reads transmission data stored in a transmission buffer area provided in a recording medium such as the RAM 1214, the storage device 1224, the DVD-ROM 1227, or the IC card, transmits the read transmission data to the network, or writes reception data received from the network to a reception buffer area or the like provided on the recording medium.

In addition, the CPU 1212 may cause the RAM 1214 to read all or a necessary portion of a file or database stored in an external recording medium such as the storage device 1224, the DVD drive 1226 (DVD-ROM 1227), an IC card, or the like, and may execute various types of processing on data on the RAM 1214. Next, the CPU 1212 may write back the processed data to the external recording medium.

Various types of information such as various types of programs, data, tables, and databases may be stored in a recording medium and subjected to information processing. The CPU 1212 may execute various types of processing on the data read from the RAM 1214, including various types of operations, information processing, condition determination, conditional branching, unconditional branching, information retrieval/replacement, and the like, which are described throughout the disclosure and specified by a command sequence of a program, and writes back the results to the RAM 1214. In addition, the CPU 1212 may search for information in a file, a database, or the like in the recording medium. For example, in a case where a plurality of entries each having the attribute value of the first attribute associated with the attribute value of the second attribute is stored in the recording medium, the CPU 1212 may search for an entry in which the attribute value of the first attribute matches the specified condition from the plurality of entries, read the attribute value of the second attribute stored in the entry, and acquire the attribute value of the second attribute associated with the first attribute satisfying the predetermined condition.

The program or software module described above may be stored in a computer-readable storage medium on the computer 1200 or near the computer 1200. Furthermore, a recording medium such as a hard disk or a RAM provided in a server system connected to a dedicated communication network or the Internet can be used as a computer-readable storage medium, thereby providing a program to the computer 1200 via the network.

The blocks in the flowcharts and block diagrams in the present embodiment may represent stages of a process in which an operation is executed or “units” of a device that are responsible for performing the operation. Certain stages and “units” may be implemented by a dedicated circuit, a programmable circuit supplied with computer-readable instructions stored on a computer-readable storage medium, and/or a processor supplied with computer-readable instructions stored on a computer-readable storage medium. Dedicated circuits may include digital and/or analog hardware circuits, and may include integrated circuits (ICs) and/or discrete circuits. The programmable circuit may include reconfigurable hardware circuits including AND, OR, XOR, NAND, NOR, and other logical operations, flip-flops, registers, and memory elements, such as field programmable gate arrays (FPGAs) and programmable logic arrays (PLAs).

The computer-readable storage medium may include any tangible device capable of storing instructions for execution by a suitable device, such that a computer-readable storage medium having instructions stored thereon includes an article of manufacture including instructions that may be executed to create means for performing the operations specified in the flowcharts or block diagrams. Examples of the computer-readable storage medium may include an electronic storage medium, a magnetic storage medium, an optical storage medium, an electromagnetic storage medium, and a semiconductor storage medium. More specific examples of the computer-readable storage medium may include a floppy (registered trademark) disk, a diskette, a hard disk, a random access memory (RAM), a read only memory (ROM), an erasable programmable read only memory (EPROM or flash memory), an electrically erasable programmable read only memory (EEPROM), a static random access memory (SRAM), a compact disc read only memory (CD-ROM), a digital versatile disc (DVD), a Blu-ray (registered trademark) disc, a memory stick, and an integrated circuit card.

The computer-readable instructions may include either source code or object code written in any combination of one or more programming languages, including assembler instructions, instruction-set-architecture (ISA) instructions, machine instructions, machine-dependent instructions, microcode, firmware instructions, state-setting data, or an object oriented programming language such as Smalltalk, JAVA (registered trademark), C++, or the like, and conventional procedural programming languages, such as the “C” programming language or similar programming languages.

The computer-readable instructions may be provided, locally or via a wide area network (WAN) such as a local area network (LAN) or the Internet, to a processor of a general-purpose computer, a special-purpose computer, or another programmable data processing apparatus, or to a programmable circuit, such that the processor of the general-purpose computer, the special-purpose computer, or another programmable data processing apparatus, or the programmable circuit executes the computer-readable instructions to generate means for performing the operations specified in a flowchart or block diagram. Examples of the processor include a computer processor, a processing unit, a microprocessor, a digital signal processor, a controller, and a microcontroller.

Although the disclosure has been described using the embodiments, the technical scope of the disclosure is not limited to the scope described in the embodiments. It is apparent to those skilled in the art that various modifications or improvements can be made to the above embodiments. It is apparent from the description of the claims that embodiments to which such modifications or improvements have been made may also be included in the technical scope of the disclosure.

It should be noted that, in the apparatuses, systems, programs, and methods shown in the claims, the specification, and the drawings, the execution order of each processing such as operations, procedures, steps, and stages is not explicitly specified as “before,” “prior to,” and the like, and, unless the output of a previous process is used in subsequent processing, the processing procedures may be implemented in any order. Even if the operations in the operation flow in the claims, the specification, and the drawings are described using expressions such as “first” and “next” for convenience, this does not mean that it is essential to perform them in this order.

### Other Embodiment 1

As another embodiment, the robot 100 may be applied to a robot mounted on a device such as an electrical appliance, a computer, an automobile, a motorcycle, or a toy, or the robot 100 may control a device such as an electrical appliance, a computer, an automobile, a motorcycle, or a toy. Furthermore, the robot 100 may be mounted on a stuffed toy, a doll, or the like, or may be applied to a control device connected to a control target device (speaker or camera) mounted on a stuffed toy, a doll, or the like in a wireless or wired manner.

Other embodiments are specifically configured as follows. For example, the robot 100 may be applied to control of the conversational scheduler. In a case where the robot 100 is applied to control of a conversational scheduler, for example, the robot 100 may be configured to include, as the control target 252, a display panel, a speaker, an LED, a bell, and the like used for an alarm clock, for notifying a schedule to a user.

The robot 100 executes processing of setting a behavior plan (schedule) of the user according to the preference of the user, the situation of the user, and the reaction of the user by Steps 1 to 5-2 below.

(Step 1) The robot 100 acquires the state of the user 10, the emotion value of the user 10, the emotion value of the robot 100, and the history data 222. Specifically, processing similar to above steps S100 to S103 is performed to acquire the state of the user 10, the emotion value of the user 10, the emotion value of the robot 100, and the history data 222.

(Step 2) The robot 100 acquires a preference of the user 10 regarding a place, a sport, a hobby, and the like.

Specifically, the behavior determination unit 236 determines, as a behavior of the robot 100, an utterance of a question content regarding a favorite place, a favorite sport, a favorite hobby, or the like to the user 10, and the behavior control unit 250 controls the control target 252 and makes an utterance to the user 10 to ask a question about a favorite place, a favorite sport, a favorite hobby, or the like. The user state recognition unit 230 recognizes a preference of the user 10 regarding a place, a sport, a hobby, and the like based on the information (for example, the user's answer) analyzed by the sensor module unit 210.

It should be noted that the behavior control unit 250 is not limited to a case of making an utterance to the user 10 to directly ask a question about a favorite place, a favorite sport, or a favorite hobby. For example, the behavior control unit 250 may make an utterance to the user 10 of indirectly ask a question about a favorite place, a favorite sport, and a favorite hobby.

(Step 3) The robot 100 determines a behavior plan to be proposed to the user 10.

Specifically, the behavior determination unit 236 adds a predetermined fixed sentence to the text representing the preference of the user 10 regarding the places, sports, hobbies, and the like, the emotion of the user 10, the emotion of the robot 100, and the content stored in the history data 222, and inputs the text to a sentence generation model, thereby acquiring the content of preference of the user 10. For example, fixed sentences such as “WHERE IS YOUR RECOMMENDED PLACE?”, “WHAT IS YOUR RECOMMENDED SPORT?”, “WHAT IS YOUR RECOMMENDED DISH?”, “WHAT IS YOUR RECOMMENDED MUSIC?”, “WHAT IS YOUR RECOMMENDED MOVIE?”, and “WHERE IS YOUR RECOMMENDED TRAVEL DESTINATION?” may be added and input to a sentence generation model to acquire the recommended content of the user 10. At this time, it is possible to propose a behavior plan suitable for the user 10 by taking into consideration not only the preferences regarding the places, sports, hobbies, and the like of the user 10 but also the emotions of the user 10 and the history data 222. Furthermore, by taking into consideration the emotion of the robot 100, it is possible to make the user 10 feel that the robot 100 has an emotion.

(Step 4) The robot 100 proposes the behavior plan determined in Step 3 to the user 10 and acquires the reaction of the user 10.

Specifically, the behavior determination unit 236 determines, as a behavior of the robot 100, an utterance to the user 10 for proposing a behavior plan, and the behavior control unit 250 controls the control target 252 and makes an utterance to the user 10 for proposing the behavior plan. For example, in a case of recognizing that the user 10 likes mountain climbing, the robot 100 proposes “SHALL WE GO TO SEE THE FIRST SUNRISE OF THE YEAR ON MT. FUJI ON NEW YEAR'S DAY NEXT YEAR?” and acquires a reaction of the user 10. Furthermore, for example, in a case of recognizing that the user 10 likes baseball, the robot 100 proposes “SHALL WE GO TO WATCH BASEBALL ON THE WEEKEND?” and acquires the reaction of the user 10. Furthermore, for example, in a case where it is recognized that the user 10 likes a specific actor A, the robot 100 proposes “SHALL WE GO TO SEE A MOVIE STARRING ACTOR A TONIGHT?” and acquires the reaction of the user 10. The user state recognition unit 230 recognizes the state of the user 10 based on the information analyzed by the sensor module unit 210, and the emotion determination unit 232 determines an emotion value indicating the emotion of the user 10 based on the information analyzed by the sensor module unit 210 and the state of the user 10 recognized by the user state recognition unit 230. The behavior determination unit 236 determines whether the reaction of the user 10 is positive based on the state of the user 10 recognized by the user state recognition unit 230 and the emotion value indicating the emotion of the user 10, and determines whether to execute processing of setting a behavior plan proposed to the user 10 or to propose another behavior plan to the user 10 as the behavior of the robot 100.

(Step 5-1) In a case where the reaction of the user 10 is positive, the robot 100 executes processing of setting a proposed behavior plan.

Specifically, in a case where it is determined that the processing of setting the behavior plan proposed to the user 10 is executed as the behavior of the robot 100, the behavior control unit 250 controls the display panel, the speaker, the LED, the bell, and the like that are the control target 252 to execute the setting of the behavior plan proposed to the user 10.

Furthermore, the robot 100 may change the notification mode when notifying the user 10 of the behavior plan according to the state of the user 10 and the emotion of the user.

Specifically, the volume of the alarm sound output from the speaker and the type of the notification sound may be changed, and the color of turning on or blinking the LED may be changed.

(Step 5-2) In a case where the reaction of the user 10 is not positive, the robot 100 determines another behavior plan to be proposed to the user 10.

Specifically, in a case where it is determined to propose another behavior plan to the user 10 as the behavior of the robot 100, the behavior determination unit 236 adds fixed sentences such as “ARE THERE ANY OTHER RECOMMENDED PLACES?”, “ARE THERE ANY OTHER RECOMMENDED SPORTS?”, “ARE THERE ANY OTHER RECOMMENDED DISHES?”, “ARE THERE ANY OTHER RECOMMENDED MUSIC?”, “ARE THERE ANY OTHER RECOMMENDED MOVIES?”, and “ARE THERE ANY OTHER RECOMMENDED TRAVEL DESTINATIONS?” to text representing preferences of the user 10 regarding a place, sports, hobbies, and the like, emotions of the user 10, emotions of the robot 100, and content stored in the history data 222, inputs the text to a sentence generation model, and acquires the recommended content regarding the behavior plan. Then, the processing returns to Step 4 described above, and the processing of Steps 4 to 5-2 described above is repeated until it is determined to execute the processing of setting the behavior plan proposed to the user 10.

In this regard, the robot 100 can execute processing of setting a behavior plan according to the preference of the user 10, the situation of the user 10, and the reaction of the user 10.

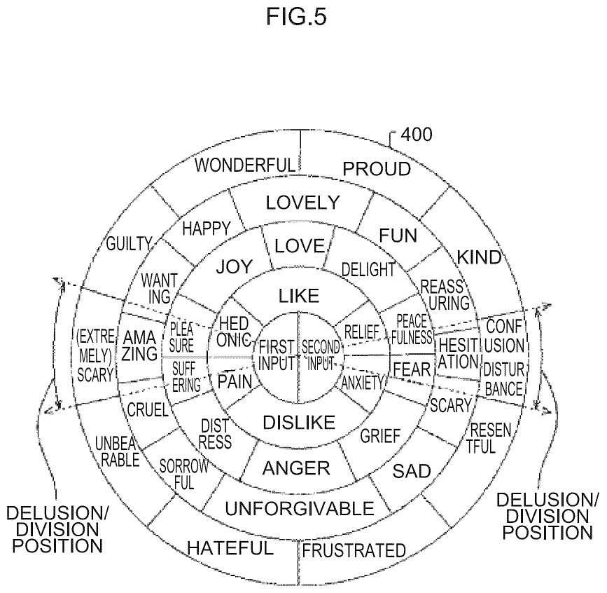

The emotion determination unit 232 may determine the emotion of the user according to a specific mapping. Specifically, the emotion determination unit 232 may determine the emotion of the user based on an emotion map (see FIG. 5) that is a specific mapping.

FIG. 5 is a diagram illustrating an emotion map 400 on which a plurality of emotions is mapped. In the emotion map 400, emotions are arranged concentrically in a radial manner from the center. The closer to the center of the concentric circles, the more primitive emotions are arranged. On the outer side of the concentric circles, emotions representing states and behaviors arising from a mental state are arranged. The emotion is a concept including an affective state and psychological conditions. On the left side of the concentric circle, emotions generated from reactions generally occurring in the brain are arranged. On the right side of the concentric circle, emotions induced by situation determination are generally arranged. In the upward and downward directions of the concentric circles, emotions generated from reactions generally occurring in the brain and induced by situation determination are arranged.

Furthermore, the emotion of “COMFORT” is arranged on the upper side of the concentric circle, and the emotion of “DISCOMFORT” is arranged on the lower side. In this regard, in the emotion map 400, a plurality of emotions is mapped based on a structure in which emotions are generated, and emotions that are likely to occur at the same time are mapped close to each other.

(1) For example, in a case where the emotion engine, which is the emotion determination unit 232 of the robot 100, detects the emotion in approximately 100 msec, the determination of the reaction operation (for example, backchannel response) of the robot 100 may be set at a timing at which the frequency is at least similar to the detection frequency (100 msec) of the emotion engine, or may be set at a timing earlier than the detection frequency. The detection frequency of the emotion engine may be interpreted as a sampling rate.

By detecting an emotion in approximately 100 msec and performing a reaction operation (for example, backchannel response) in immediate association therewith, it is possible to eliminate unnatural backchannel responses and realize a natural, context-aware interaction. The robot 100 performs a reaction operation (such as a backchannel response) according to the direction and the degree (intensity) of the mandala of the emotion map 400. Note that the detection frequency (sampling rate) of the emotion engine is not limited to 100 ms, and may be changed according to the situation (such as when playing sports), the age of the user, or the like.

(2) The direction of the emotion and the intensity of the degree thereof may be preset in correspondence with the emotion map 400, and the movement of the backchannel response and the strength of the backchannel response may be set. For example, in a case where the robot 100 feels stability or relief, the robot 100 continues to listen to the conversation while nodding. In a case where the robot 100 feels anxiety, hesitation, or suspicion, the robot 100 may tilt its head or stop shaking its head.

These emotions are distributed in the three o'clock direction of the emotion map 400 and usually move back and forth between the areas of relief and anxiety. In the right half of the emotion map 400, situation recognition is superior to internal sensation, and thus gives a calm impression.

(3) In a case where the robot 100 feels pleasant feeling from being praised, a filler such as “AH” may be inserted before the line, and in a case where it feels a twinge from receiving harsh words, a filler such as “UGH!” may be inserted before the line. It is also acceptable that the robot 100 includes a physical reaction such as a movement of crouching while uttering “UGH!”. These emotions are distributed around the 9 o'clock direction on the emotion map 400.

(4) In the left half of the emotion map 400, internal sensation (reaction) is superior to situation recognition. Therefore, an impression of unintentional reaction can be given.

In a case where the robot 100 has a favorable feeling in situation recognition while having an internal feeling (reaction) of satisfaction, the robot 100 may nod deeply while observing the other party and may also utter “UH-HUH”. In this regard, the robot 100 may generate a balanced favorable feeling to the other party, that is, a behavior such as acceptance or tolerance to the other party. Such emotions are distributed around the 12 o'clock direction on the emotion map 400.

Conversely, even in the situation recognition while the robot 100 remembers the internal feeling (reaction) of unpleasant feeling, the robot 100 may shake its head sideways when feeling antipathy, and may turn red the LED of the eye and look at the other party when feeling hatred. Such emotions are distributed around the 6 o'clock direction on the emotion map 400.

(5) Since the inner side of the emotion map 400 represents the inside of the mind and the outer side of the emotion map 400 represents a behavior, the emotion is more visible (appears in the behavior) toward the outer side of the emotion map 400.

(6) In a case of listening to a person while feeling a sense of relief distributed around the 3 o'clock position of the emotion map 400, the robot 100 may merely give a slight nod accompanied by uttering “HM-HM”. However, when the emotion shifts toward love around the 12 o'clock position, the robot 100 may give a strong, deep nod.

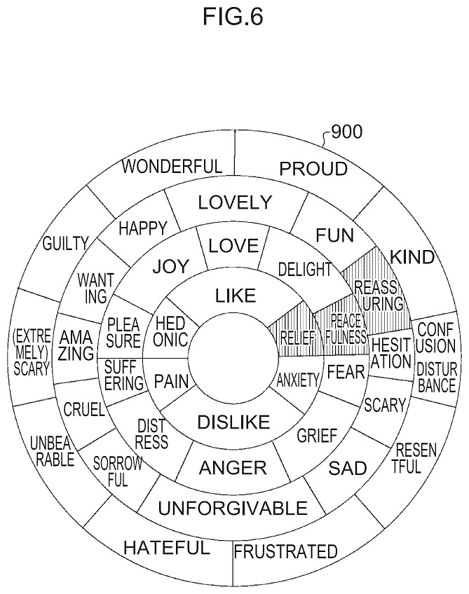

The emotion determination unit 232 inputs the information analyzed by the sensor module unit 210 and the recognized state of the user 10 to a neural network learned in advance, acquires an emotion value indicating each emotion indicated in the emotion map 400, and determines the emotion of the user 10. This neural network is learned in advance based on a plurality of pieces of learning data that is a combination of the information analyzed by the sensor module unit 210 and the recognized state of the user 10 and the emotion value indicating each emotion indicated in the emotion map 400. Furthermore, in this neural network, as in an emotion map 900 illustrated in FIG. 6, emotions arranged close to each other are learned to have close values. FIG. 6 illustrates an example in which a plurality of emotions such as “RELIEF”, “PEACEFULNESS”, and “REASSURING” has similar emotion values.

Furthermore, the emotion determination unit 232 may determine the emotion of the robot 100 according to a specific mapping. Specifically, the emotion determination unit 232 inputs the information analyzed by the sensor module unit 210, the state of the user 10 recognized by the user state recognition unit 230, and the state of the robot 100 to a neural network learned in advance, acquires an emotion value indicating each emotion indicated in the emotion map 400, and determines the emotion of the robot 100. This neural network is learned in advance based on a plurality of pieces of learning data that is a combination of the information analyzed by the sensor module unit 210, the recognized state of the user 10, the state of the robot 100, and the emotion value indicating each emotion indicated in the emotion map 400. For example, a neural network is learned based on the learning data representing that the emotion value of “HAPPY” becomes “3” in a case where it is recognized from the output of a touch sensor (not illustrated) that the robot 100 is being stroked by the user 10, and learning data representing that the emotion value of “ANGER” becomes “3” in a case where it is recognized from the output of an acceleration sensor (not illustrated) that the robot 100 is being hit by the user 10. Furthermore, in this neural network, as in an emotion map 900 illustrated in FIG. 6, emotions arranged close to each other are learned to have close values.

The behavior determination unit 236 adds a fixed sentence for inquiry about the behavior content of the robot corresponding to the behavior of the user to the text representing the behavior of the user, the emotion of the user, and the emotion of the robot, and inputs the text to a sentence generation model having the interaction function, thereby generating the behavior content of the robot.

For example, the behavior determination unit 236 acquires text representing the state of the robot 100 from the emotion of the robot 100 determined by the emotion determination unit 232 using the emotion table as illustrated in Table 1. Here, in the emotion table, an index number is assigned to each emotion value for each type of emotion, and text representing the state of the robot 100 is stored for each index number.

In a case where the emotion of the robot 100 determined by the emotion determination unit 232 corresponds to the index number “2”, text “VERY FUN STATE” is obtained. Note that, in a case where the emotion of the robot 100 corresponds to a plurality of index numbers, a plurality of pieces of text representing the state of the robot 100 is obtained.

Furthermore, the emotion table as illustrated in Table 2 is prepared for the emotion of the user 10.

Here, in a case where the behavior of the user speaks “I LIKE CLIMBING MT. FUJI”, the emotion of the robot 100 is the index number “2”, and the emotion of the user 10 is the index number “3”, “THE ROBOT IS IN A VERY FUN STATE. THE USER IS IN AN ORDINARILY FUN STATE. THE USER SAID, ‘I LIKE CLIMBING MT. FUJI,’ TO THE ROBOT. AS THE ROBOT, HOW WOULD YOU REPLY?” is input to the sentence generation model, and the behavior content of the robot is acquired. The behavior determination unit 236 determines a behavior of the robot from the behavior content.

TABLE 1

| Index number | Emotion type | Emotion value | State of robot |
|---|---|---|---|
| 1 | Fun | 5 | Extremely fun state |
| 2 | Fun | 4 | Very fun state |
| 3 | Fun | 3 | Ordinarily fun state |
| 4 | Fun | 2 | Slightly fun state |
| 5 | Fun | 1 | Just slightly fun state |
| . . . | . . . | . . . | . . . |

TABLE 2

| Index number | Emotion type | Emotion value | State of user |
|---|---|---|---|
| 1 | Fun | 5 | Extremely fun state |
| 2 | Fun | 4 | Very fun state |
| 3 | Fun | 3 | Ordinarily fun state |
| 4 | Fun | 2 | Slightly fun state |
| 5 | Fun | 1 | Just slightly fun state |
| . . . | . . . | . . . | . . . |

In this regard, the behavior determination unit 236 determines the behavior content of the robot 100 according to the state related to the emotion of the robot 100 determined in advance for each type of the emotion of the robot 100 and for each strength of the emotion, and the behavior of the user 10. In this embodiment, the utterance content of the robot 100 in a case where an interaction with the user 10 is performed can be branched according to the state related to the emotion of the robot 100. That is, since the robot 100 can change the behavior of the robot according to the index number according to the emotion of the robot, the user has an impression that the robot possesses emotions, and is promoted to take a behavior such as talking to the robot.

Furthermore, the behavior determination unit 236 may generate the behavior content of the robot by adding a fixed sentence for asking a question about the behavior content of the robot corresponding to the behavior of the user and inputting the text to the sentence generation model having the interaction function after adding not only the text representing the behavior of the user, the emotion of the user, and the emotion of the robot but also the text representing the content of the history data 222. As a result, the robot 100 can change the behavior of the robot according to the history data representing the emotion and behavior of the user, and thus, the user has an impression that the robot has personality, thereby promoting the user to take behaviors such as talking to the robot. Furthermore, the history data may further include the emotion and behavior of the robot.

Furthermore, the emotion determination unit 232 may determine the emotion of the robot 100 based on the behavior content of the robot 100 generated by the sentence generation model. Specifically, the emotion determination unit 232 inputs the behavior content of the robot 100 generated by the sentence generation model to the neural network learned in advance, acquires the emotion value indicating each emotion indicated in the emotion map 400, integrates the acquired emotion value indicating each emotion and the emotion value indicating each emotion of the current robot 100, and updates the emotion of the robot 100. For example, the acquired emotion value indicating each emotion and the emotion value indicating each emotion of the current robot 100 are averaged and integrated. This neural network is learned in advance based on a plurality of pieces of learning data that is a combination of text representing the behavior content of the robot 100 generated by the sentence generation model and the emotion value indicating each emotion illustrated in the emotion map 400.

For example, in a case where, as the content of a behavior of the robot 100 generated by the sentence generation model, utterance content of the robot 100, “THAT'S GOOD. YOU WERE LUCKY”, is obtained, when text representing the utterance content is input to a neural network, a high value is obtained as the emotion value of the emotion “HAPPY” and the emotion of the robot 100 is updated such that the emotion value of the emotion “HAPPY” becomes high.

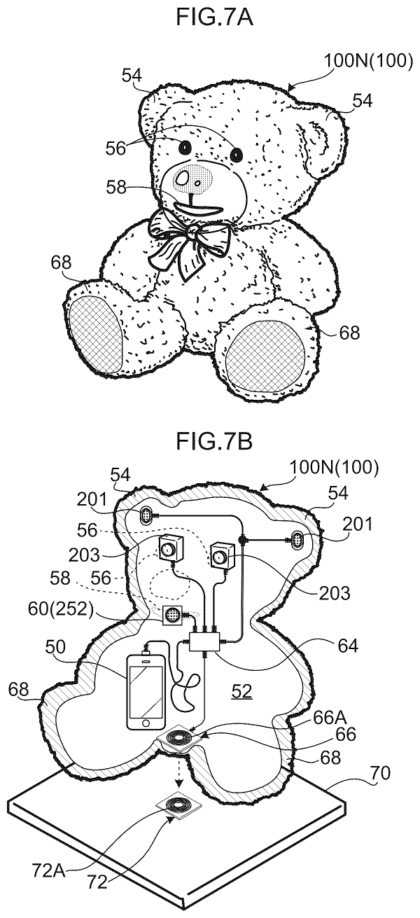

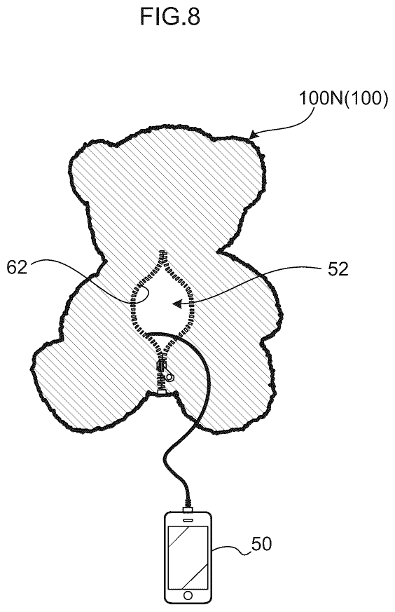

The robot 100 may be mounted on a stuffed toy, or may be applied to a control device connected to a control target device (speaker or camera) mounted on the stuffed toy in a wireless or wired manner. In this case, specifically, it is configured as follows. For example, the robot 100 may be applied to a companion (specifically, a stuffed toy 100N illustrated in FIGS. 7 and 8) who advances an interaction with the user 10 based on information regarding daily life while spending daily life with the user 10 or provides information matching the hobby of the user 10.

In the present embodiment (another embodiment), an example in which the control portion of the robot 100 is applied to a smartphone 50 will be described.

The stuffed toy 100N having a function as an input/output device of the robot 100 has a configuration in which the smartphone 50, which functions as a control portion of the robot 100, is detachably attached, and the input/output device and the accommodated smartphone 50 are connected inside the stuffed toy 100N.

As illustrated in FIG. 7(A), the stuffed toy 100N has the shape of a bear covered with a soft cloth fabric in the present embodiment (embodiment mounted on the stuffed toy). As illustrated in FIG. 7(B), in a space portion 52 formed inside the stuffed toy, as input/output devices, the microphone 201 (see FIG. 2) of the sensor unit 200 is disposed in a portion corresponding to an ear 54, the 2D camera 203 of the sensor unit 200 is disposed in a portion corresponding to eyes 56 (see FIG. 2), and a speaker 60, which constitutes a part of the control target 252 (see FIG. 2), is disposed in a portion corresponding to a mouth 58. The microphone 201 and the speaker 60 are not necessarily separated from each other, and may be an integrated unit. In the case of the unit, the unit may be disposed at a position where the utterance can be heard naturally, such as the position of the nose of the stuffed toy 100N. Although the case where the stuffed toy 100N has an animal shape has been described as an example, it is not limited thereto. The stuffed toy 100N may have a shape of a specific character.

The smartphone 50 has a function as the sensor module unit 210, a function as the storage unit 220, a function as the user state recognition unit 230, a function as the emotion determination unit 232, a function as the behavior recognition unit 234, a function as the behavior determination unit 236, a function as the memory control unit 238, a function as the behavior control unit 250, and a function as the communication processing unit 280 illustrated in FIG. 2.

As illustrated in FIG. 8, a fastener 62 is attached to apart (for example, the back portion) of the stuffed toy 100N, and the outside and the space portion 52 communicate with each other by opening the fastener 62.

Here, the smartphone 50 is accommodated in the space portion 52 from the outside and is connected to each input/output device in a USB via a USB hub 64 (see FIG. 7(B)), such that a function equivalent to that of the robot 100 illustrated in FIG. 1 can be provided.

In addition, a non-contact power receiving plate 66 is connected to the USB hub 64. Power receiving coil 66A is incorporated in the power receiving plate 66. The power receiving plate 66 is an example of a wireless power receiving unit that receives wireless power supply.

The power receiving plate 66 is disposed near root portions 68 of both feet of stuffed toy 100N, and is located closest to a mounting base 70 when the stuffed toy 100N is placed on the mounting base 70. The mounting base 70 is an example of an external wireless power transmitter.

The stuffed toy 100N placed on the mounting base 70 can be appreciated as an ornament in a natural state.

In addition, this root portion is formed to be thinner than the surface layer thickness of the stuffed toy 100N in other parts, and is held in a state closer to the mounting base 70.

The mounting base 70 includes a charging pad 72. Power transmitting coil 72A is incorporated in the charging pad 72. When the power transmitting coil 72A transmits a signal to search the power receiving coil 66A of the power receiving plate 66, and the power receiving coil 66A is found, a current flows through the power transmitting coil 72A to generate a magnetic field, and the power receiving coil 66A reacts to the magnetic field to start electromagnetic induction. As a result, current flows through the power receiving coil 66A, and power is stored in a battery (not illustrated) of the smartphone 50 via the USB hub 64.

That is, since the smartphone 50 is automatically charged by placing the stuffed toy 100N as an ornament on the mounting base 70, it is not necessary to take out the smartphone 50 from the space portion 52 of the stuffed toy 100N for charging.

In the present embodiment (the embodiment mounted on a stuffed toy), the smartphone 50 is accommodated in the space portion 52 of the stuffed toy 100N and connected by wire (USB connection), but it is not limited thereto. For example, a control device having a wireless function (for example, “Bluetooth (registered trademark)”) may be accommodated in the space portion 52 of the stuffed toy 100N, and the control device may be connected to the USB hub 64. In this case, the smartphone 50 and the control device wirelessly communicate with each other without inserting the smartphone 50 into the space portion 52, and the external smartphone 50 is connected to each input/output device via the control device, such that a function equivalent to that of the robot 100 illustrated in FIG. 1 can be provided. Furthermore, a control device accommodated in the space portion 52 of the stuffed toy 100N and an external smartphone 50 may be connected to each other in a wired manner.

Furthermore, in the present embodiment (embodiment mounted on a stuffed toy), the bear-shaped stuffed toy 100N has been exemplified, but may be another animal, a doll, or a shape of a specific character. Further, the clothes may be changeable. Furthermore, the material of the skin is not limited to the cloth fabric, and may be other materials such as soft vinyl, but is preferably a soft material.

Furthermore, a monitor may be attached to the skin of the stuffed toy 100N, and the control target 252 that provides information to the user 10 through vision may be added. For example, the eyes 56 may be used as a monitor to express happiness, anger, grief, and enjoyment by images displayed in the eyes, or a window through which the monitor of the built-in smartphone 50 is visible may be provided in the abdomen. Furthermore, the eyes 56 may be used as a projector to express happiness, anger, grief, and enjoyment by an image projected on a wall surface.

According to another embodiment, the existing smartphone 50 is placed in the stuffed toy 100N, and the camera 203, the microphone 201, the speaker 60, and the like are extended from there to appropriate positions via a USB connection.

Further, for wireless charging, the smartphone 50 and the power receiving plate 66 are connected via the USB, and the power receiving plate 66 is disposed to be as far as possible from the inside of the stuffed toy 100N.

In order to use the wireless charging of the smartphone 50, it is necessary to dispose the smartphone 50 on the outside as much as possible when viewed from the inside of the stuffed toy 100N, and the stuffed toy 100N is rough when touched from the outside.

Therefore, the smartphone 50 is disposed at the center of the stuffed toy 100N as much as possible, and the wireless charging function (power receiving plate 66) is disposed outside as viewed from the inside of the stuffed toy 100N as much as possible. The camera 203, the microphone 201, the speaker 60, and the smartphone 50 receive wireless power supply via the power receiving plate 66.

### Other Embodiment 2

As another embodiment, the robot 100 may be applied to a robot mounted on a device such as an electrical appliance, a computer, an automobile, a motorcycle, or a toy, or the robot 100 may control a device such as an electrical appliance, a computer, an automobile, a motorcycle, or a toy. Furthermore, the robot 100 may be mounted on a stuffed toy, or may be applied to a control device connected to a control target device (speaker or camera) mounted on the stuffed toy in a wireless or wired manner. Other embodiments are specifically configured as follows. For example, the robot 100 may be applied to control the event guidance system.

The event guidance system has a function (event guidance function) of guiding the user 10 to an event. In other words, the event guidance system has a conversation with the user 10 while observing the preference, situation, and reaction of the user 10. Then, the event guidance system autonomously guides the user 10 to the event. The guidance of the event includes, for example, guiding the user 10 to a specific event. The guidance of the event includes a case of guiding purchase of event tickets and a case of urging purchase of event tickets. For example, a method of purchasing a ticket is displayed on a display device 1218 of the robot 100. The method of purchasing the ticket includes a release period, a release place, and a purchase method (which may include a payment method) of the ticket. In addition, the speaker of the robot 100 may provide guidance by voice on a release period, a release place, and a purchase method of the ticket.

The information related to the guided event, that is, the event-related information may be further guided to the user 10. The event-related information includes, for example, past publications and sales of event parties (organizers, co-hosts, supporters, performers, artists, and the like), information sources on the Internet, and the like. The event-related information includes, for example, transportation means to a venue of the event, and guidance on means of travel.

Furthermore, for example, information regarding ticket issuance for an event may be stored in the storage device 1224 or the like, and the information regarding ticket issuance may be transmitted to the ticket issuing device. For example, in a state where the robot 100 is near the ticket issuing device, the robot 100 transmits information regarding the ticket issuance to the ticket issuing device. Then, the ticket issuing device that has received the information regarding ticket issuance automatically issues a ticket. As a result, the user 10 is spared the trouble of operating the ticket issuing device.

Furthermore, the robot 100 may include a ticket function unit. This ticket function unit is a part that functions as a ticket for an event guided by the event guidance system to the user 10. For example, the fact that the user 10 completes the registration of participation in the event or the payment of the fee is stored in the storage device 1224 or the like. Then, the ticket function unit wirelessly communicates with a check-in terminal (ticket inspection device) at the event venue, such that it is confirmed that the user 10 is an authorized entering person, and the user 10 can enter the event venue.

The type of the event is not limited. Examples thereof include a movie, a play, a music concert, a lecture, an exposition, an exhibition, a trade show, various consultation meetings, a product exhibition, a goods sale, and the like. These events include paid events and free events.

The robot 100 as the ticket guidance system includes various functional units for executing the above operations. Hereinafter, as an example, a case where the robot 100 exhibits an event guidance function will be described as an example.

The robot 100 executes processing of selecting an event to be guided to the user 10 according to the preference of the user 10, the situation of the user 10, and the reaction of the user 10 by Steps 1 to 5-2 below.

(Step 1) The robot 100 acquires the state of the user 10, the emotion value of the user 10, the emotion value of the robot 100, and the history data 222. Specifically, processing similar to above steps S100 to S103 is performed to acquire the state of the user 10, the emotion value of the user 10, the emotion value of the robot 100, and the history data 222.

(Step 2) The robot 100 acquires a preference of the user 10 regarding the type of the event.

Specifically, the behavior determination unit 236 determines, as a behavior of the robot 100, an utterance to the user 10 to ask a question about a preference regarding a type of an event, and the behavior control unit 250 controls the control target 252 and makes an utterance to the user 10 to ask a question about the preference regarding the type of the event. The user state recognition unit 230 recognizes the preference of the user 10 regarding the type of the event based on the information (for example, the user's answer) analyzed by the sensor module unit 210.

(Step 3) The robot 100 determines a specific example of an event to be proposed to the user 10.

Specifically, the behavior determination unit 236 adds a fixed sentence such as “AT THIS TIME, WHAT IS THE TYPE OF EVENT RECOMMENDED TO THE USER?” to the text representing the preference of the user 10 regarding the type of event, the emotion of the user 10, the emotion of the robot 100, and the content stored in the history data 222, inputs the text to a sentence generation model, and acquires the recommended content regarding the type of event. At this time, it is possible to propose a specific example of an event suitable for the user 10 by taking into consideration not only the preference regarding the type of the event of the user 10 but also the emotion of the user 10 and the history data 222. Furthermore, by taking into consideration the emotion of the robot 100, it is possible to make the user 10 feel that the robot 100 has an emotion. More specifically, for example, in the case of a movie, the preferred field of the user 10 is whether it is a foreign movie or a Japanese movie, and also is any one of the following: Mystery, Suspense, Horror, Action, SF, Adventure, Fantasy, Comedy, Documentary, and Animation.

(Step 4) The robot 100 proposes a specific example of the event determined in Step 3 to the user 10 and acquires the reaction of the user 10.

Specifically, the behavior determination unit 236 determines, as a behavior of the robot 100, an utterance to the user 10 for proposing a specific example of an event, and the behavior control unit 250 controls the control target 252 and makes an utterance to the user 10 for proposing the specific example of the event. The user state recognition unit 230 recognizes the state of the user 10 based on the information analyzed by the sensor module unit 210, and the emotion determination unit 232 determines an emotion value indicating the emotion of the user 10 based on the information analyzed by the sensor module unit 210 and the state of the user 10 recognized by the user state recognition unit 230. The behavior determination unit 236 determines whether the reaction of the user 10 is positive based on the state of the user 10 recognized by the user state recognition unit 230 and the emotion value indicating the emotion of the user 10, and determines whether to execute processing of guiding the user 10 to a specific example of an event proposed to the user 10 or to propose a specific example of another event to the user 10 as the behavior of the robot 100.

(Step 5-1) In a case where the reaction of the user 10 is positive, the robot 100 executes processing of guiding the user 10 to a specific example of the proposed event.

Specifically, in a case where it is determined to execute processing of guiding a specific example of the event proposed to the user 10 as the behavior of the robot 100, the behavior control unit 250 controls various functional units which are the control target 252 to execute the proposal of the specific example to the user 10. More specifically, the display device 1218 displays a name of an event to be guided, a location and date and time of holding (including screening and performance), participation directions, a fee, and the like.

(Step 5-2) In a case where the reaction of the user 10 is not positive, the robot 100 determines a specific example of another event to be proposed to the user 10.

Specifically, in a case where it is determined to propose a specific example of another event to the user 10 as the behavior of the robot 100, the behavior determination unit 236 adds a fixed sentence such as “ARE THERE ANY OTHER TYPES OF EVENTS RECOMMENDED TO THE USER?” to the text representing the preference of the user 10 regarding the type of event, the emotion of the user 10, the emotion of the robot 100, and the content stored in the history data 222, inputs the text to a sentence generation model, and acquires the recommended content regarding the type of event. Then, the processing returns to Step 4 described above, and the processing of Steps 4 to 5-2 described above is repeated until it is determined to execute the processing of guiding a specific example of the event proposed to the user 10.

In this regard, the robot 100 can execute processing of guiding specific examples of the event according to the preference of the user 10, the situation of the user 10, and the reaction of the user 10.

With regard to the above embodiments, the following supplementary notes are further disclosed.

### Supplementary Note 1

A behavior control system including
a user state recognition unit that recognizes a user state including a behavior of a user,
an emotion determination unit that determines an emotion of the user or an emotion of a robot,
and a behavior determination unit that determines a behavior of the robot corresponding to the user state and the emotion of the user or the emotion of the robot based on an interaction function that causes the user to interact with the robot, in which
the robot is mounted on a ticket guidance system, and
the behavior determination unit autonomously guides an event while engaging in conversation with the user while observing a preference, a situation, and a reaction of the user.

### Supplementary Note 2

The behavior control system according to Supplementary Note 1, in which the behavior determination unit presents the user with a method of purchasing a ticket for the event.

### Supplementary Note 3

The behavior control system according to Supplementary Note 1, in which the behavior determination unit stores information regarding ticket issuance in a ticket issuing device for the event.

### Supplementary Note 4

The behavior control system according to Supplementary Note 1, further including a ticket function unit that functions as a ticket for the event guided to the user by the behavior determination unit.

### Supplementary Note 5

The behavior control system according to Supplementary Note 1, in which the behavior determination unit guides the user with event-related information related to the event guided to the user.

### Supplementary Note 6

The behavior control system according to Supplementary Note 1, in which the robot is mounted on a stuffed toy or is connected to a control target device mounted on the stuffed toy in a wireless or wired manner.

### Other Embodiment 3

As another embodiment, the robot 100 may be applied to a robot mounted on a device such as an electrical appliance, a computer, an automobile, a motorcycle, or a toy, or the robot 100 may control a device such as an electrical appliance, a computer, an automobile, a motorcycle, or a toy. Furthermore, the robot 100 may be mounted on a stuffed toy, or may be applied to a control device connected to a control target device (speaker or camera) mounted on the stuffed toy in a wireless or wired manner. Other embodiments are specifically configured as follows. For example, the robot 100 may be applied to a walking assist device (AI walking assist device).

The walking assist device is a device that supports walking of a user when the user such as an elderly person or a patient with impaired legs walks, and examples thereof include a walker such as a handcart for an aged person, a cane, a clutch, and the like.

The robot 100 executes processing of determining a walking method to be proposed to the user according to the preference of the user, the situation of the user, and the reaction of the user by Steps 1 to 5-2 below. The walking method (manner of walking) described herein includes, for example, a walking pace, a pace distribution, a distance, a stride, a way of raising a foot, a hand swing width, and the like.

(Step 1) The robot 100 acquires the state of the user 10, the emotion value of the user 10, the emotion value of the robot 100, and the history data 222. Specifically, processing similar to above steps S100 to S103 is performed to acquire the state of the user 10, the emotion value of the user 10, the emotion value of the robot 100, and the history data 222.

(Step 2) The robot 100 acquires the preference of the user 10 regarding the walking pace or the pace distribution.

Specifically, the behavior determination unit 236 determines, as the behavior of the robot 100, an utterance to the user 10 to ask a question about a preference regarding the walking pace or the pace distribution, and the behavior control unit 250 controls the control target 252 and makes an utterance to the user 10 to ask the question about the walking pace or the pace distribution. The user state recognition unit 230 recognizes the preference of the user 10 regarding a walking pace and a pace distribution based on information (for example, the user's answer) analyzed by the sensor module unit 210.

(Step 3) The robot 100 determines a walking method proposed to the user 10.

Specifically, the behavior determination unit 236 adds a fixed sentence such as “AT THIS TIME, WHAT IS THE WALKING PACE OR PACE DISTRIBUTION RECOMMENDED TO THE USER?” to the text representing the preference of the user 10 regarding the walking pace or the pace distribution, the emotion of the user 10, the emotion of the robot 100, and the content stored in the history data 222, inputs the text to a sentence generation model, and acquires the recommended content regarding the walking pace or the pace distribution. At this time, it is possible to propose a reinforcement method suitable for the user 10 by taking into consideration not only the preference of the user 10 regarding the walking pace or the pace distribution but also the emotion of the user 10 and the history data 222.

Furthermore, by taking into consideration the emotion of the robot 100, it is possible to make the user 10 feel that the robot 100 has an emotion.

(Step 4) The robot 100 proposes the walking method determined in Step 3 to the user 10 and acquires the reaction of the user 10.

Specifically, the behavior determination unit 236 determines, as a behavior of the robot 100, an utterance to the user for proposing a walking method to the user 10, and the behavior control unit 250 controls the control target 252 and makes an utterance to the user 10 for proposing the walking method. The user state recognition unit 230 recognizes the state of the user 10 based on the information analyzed by the sensor module unit 210, and the emotion determination unit 232 determines an emotion value indicating the emotion of the user 10 based on the information analyzed by the sensor module unit 210 and the state of the user 10 recognized by the user state recognition unit 230.

The behavior determination unit 236 determines whether the reaction of the user 10 is positive based on the state of the user 10 recognized by the user state recognition unit 230 and the emotion value indicating the emotion of the user 10, and determines whether to execute processing of assisting a walking method proposed to the user 10 or to propose another walking method to the user 10 as the behavior of the robot 100.

(Step 5-1) In a case where the reaction of the user 10 is positive, the robot 100 executes processing of assisting a proposed walking method.

Specifically, in a case where it is determined to execute processing of assisting the walking method proposed to the user 10 as the behavior of the robot 100, the behavior control unit 250 makes an utterance to the user 10 regarding points of cautions, manners, and the like of the proposed walking method.

(Step 5-2) In a case where the reaction of the user 10 is not positive, the robot 100 determines another walking method proposed to the user 10.

Specifically, in a case where it is determined to propose another walking method to the user 10 as a behavior of the robot 100, the behavior determination unit 236 adds a fixed sentence such as “ARE THERE ANY OTHER WALKING PACE AND PACE DISTRIBUTION RECOMMENDED TO THE USER?”, to text representing a preference of the user 10 regarding a walking pace and a pace distribution, an emotion of the user 10, an emotion of the robot 100, and content stored in the history data 222, inputs the text to a sentence generation model, and acquires the recommended content regarding the walking pace and pace distribution. Then, the processing returns to Step 4 described above, and the processing of Steps 4 to 5-2 described above is repeated until it is determined to execute the processing of assisting the walking method proposed to the user 10.

In this regard, the robot 100 can execute processing of determining a walking method to be proposed to the user according to the preference of the user, the situation of the user, and the reaction of the user.

With regard to the above embodiments, the following supplementary notes are further disclosed.

### Supplementary Note 1

A behavior control system including
a user state recognition unit that recognizes a user state including a behavior of a user, an emotion determination unit that determines an emotion of the user or an emotion of a robot,
and a behavior determination unit that determines a behavior of the robot corresponding to the user state and the emotion of the user or the emotion of the robot based on a sentence generation model having an interaction function of causing the user to interact with the robot, in which
the behavior determination unit determines a walking method to be proposed to the user based on an interaction result with the user by the sentence generation model.

### Supplementary Note 2

The behavior control system according to Supplementary Note 1, in which the behavior determination unit determines a walking method to be proposed to the user based on the interaction result with the user by the sentence generation model and the emotion of the user.

### Supplementary Note 3

The behavior control system according to Supplementary Note 1 or 2, in which the robot is mounted on a stuffed toy or is connected to a control target device mounted on the stuffed toy in a wireless or wired manner.

### Other Embodiment 4

As another embodiment, the robot 100 may be applied to a robot mounted on a device such as an electrical appliance, a computer, an automobile, a motorcycle, or a toy, or the robot 100 may control a device such as an electrical appliance, a computer, an automobile, a motorcycle, or a toy. Furthermore, the robot 100 may be mounted on a stuffed toy, or the robot 100 may control a control target device (for example, a speaker or a camera) mounted on the stuffed toy. In a case where the robot 100 controls the control target device, for example, the robot 100 is connected to the control target device in a wireless or wired manner.

Other embodiments are specifically configured as follows. For example, the robot 100 may be mounted on a storage container that stores clothes, or the robot 100 may control a control target device (for example, a speaker or a camera) mounted on the storage container. In a case where the robot 100 controls the control target device, for example, the robot 100 is connected to the control target device in a wireless or wired manner. Examples of the storage container include a chest, a closet, and a wardrobe case.

The robot 100 executes processing of presenting the condition or handling of the clothes to the user based on the interaction with the user by Steps 1 to 5 below. Specifically, the robot 100 executes processing of presenting the condition or handling of the clothes to the user according to the preference of the user, the situation of the user, and the reaction of the user by Steps 1 to 5 below. In other words, the robot 100 presents the condition or handling of the clothes to the user while having a conversation while observing the preference, the situation, and the reaction of the user.

(Step 1) The robot 100 acquires the state of the user 10, the emotion value of the user 10, the emotion value of the robot 100, and the history data 222. Specifically, processing similar to above steps S100 to S103 is performed to acquire the state of the user 10, the emotion value of the user 10, the emotion value of the robot 100, and the history data 222.

The robot 100 may acquire information regarding the clothes stored in the storage container (hereinafter, referred to as clothes information) by a detection unit (for example, a sensor including a camera) provided in the storage container. Examples of the clothes information include the presence or absence of clothes, the type of clothes, the place where the clothes are stored, and the condition of the clothes (for example, whether the clothes are folded or not, the number of times the clothes have been taken in and out, and the like).

(Step 2) The robot 100 acquires the preference of the user 10 regarding the clothes.

Specifically, the behavior determination unit 236 determines, as a behavior of the robot 100, an utterance to the user 10 to ask a question about a preference regarding clothes, and the behavior control unit 250 controls the control target 252 and makes an utterance to the user 10 to ask the question about the preference regarding the clothes. The user state recognition unit 230 recognizes the preference of the user 10 regarding the clothes based on the information (for example, the user's answer) analyzed by the sensor module unit 210. Examples of preferences regarding clothes include preferences for types of clothes (for example, formal or casual), commitment to clothes, clothing style, methods of storing clothes, and frequency of cleaning clothes.

(Step 3) The robot 100 determines a presentation content to be presented to the user 10.

Specifically, the behavior determination unit 236 adds a fixed sentence such as “AT THIS CASE, WHAT IS THE PRESENTATION CONTENT RECOMMENDED TO THE USER?” to the text representing the preference of the user 10 regarding clothes, the emotion of the user 10, the emotion of the robot 100, and the content stored in the history data 222, inputs the text into the sentence generation model, and acquires recommended presentation content regarding a condition or handling of the clothes. At this time, it is possible to present presentation content suitable for the user 10 by taking into consideration not only the preference of the user 10 regarding clothes but also the emotion of the user 10 and the history data 222. Furthermore, by taking into consideration the emotion of the robot 100, it is possible to make the user 10 feel that the robot 100 has an emotion. In a case where the clothes information is acquired, the robot 100 may determine the presentation content in consideration of the clothes information. As the presentation content, specifically, for example, there is content regarding a condition of clothes or the content regarding handling of clothes. Examples of the content regarding the condition of the clothes include content indicating whether the clothes are in a state of being scattered, necessity of cleaning the clothes, and the like. The content regarding the handling of clothes includes, for example, content for prompting the user 10 to store scattered clothes in a storage container, content for prompting the user 10 to clean clothes, and the like. The state where clothes are scattered corresponds to, for example, a state where clothes are taken out of a storage container and clothes are left in a room in which the storage container is installed.

(Step 4) The robot 100 presents the presentation content determined in Step 3 to the user 10 and acquires the reaction of the user 10.

Specifically, the behavior determination unit 236 determines, as a behavior of the robot 100, an utterance to the user 10 for presenting a presentation content, and the behavior control unit 250 controls the control target 252 and makes an utterance to the user 10 for presenting the presentation content. The user state recognition unit 230 recognizes the state of the user 10 based on the information analyzed by the sensor module unit 210, and the emotion determination unit 232 determines an emotion value indicating the emotion of the user 10 based on the information analyzed by the sensor module unit 210 and the state of the user 10 recognized by the user state recognition unit 230.

The behavior determination unit 236 determines whether the reaction of the user 10 is positive based on the state of the user 10 recognized by the user state recognition unit 230 and the emotion value indicating the emotion of the user 10.

(Step 5) In a case where the reaction of the user 10 is not positive, the robot 100 determines another presentation content presented to the user 10.

Specifically, in a case where it is determined, as the behavior of the robot 100, to present another presentation content to the user 10, the behavior determination unit 236 adds a fixed sentence such as “ARE THERE ANY OTHER PRESENTATION CONTENT RECOMMENDED TO THE USER?” to the text representing the preference regarding the clothes, the emotion of the user 10, the emotion of the robot 100, and the content stored in the history data 222, inputs the text to a sentence generation model, and acquires recommended presentation content regarding the condition or the handling of the clothes. Then, the processing returns to Step 4 above, the presentation content is presented to the user 10, and the processing of Steps 4 to 5 is repeated until it is determined that the reaction of the user 10 is positive.

In this regard, the robot 100 can execute processing of presenting the condition or handling of the clothes according to the preference of the user, the situation of the user, and the reaction of the user.

With regard to the above embodiments, the following supplementary notes are further disclosed.

### Supplementary Note 1

A behavior control system including
a user state recognition unit that recognizes a user state including a behavior of a user,
an emotion determination unit that determines an emotion of the user or an emotion of a robot,
and a behavior determination unit that determines a behavior of the robot corresponding to the user state and the emotion of the user or the emotion of the robot based on a sentence generation model having an interaction function of causing the user to interact with the robot, in which
the behavior determination unit presents the user with a condition or handling of clothes based on an interaction between the user and the robot.

### Supplementary Note 2

The behavior control system according to Supplementary Note 1, in which
the behavior determination unit presents necessity of cleaning of the clothes to the user as the condition based on an interaction between the user and the robot.

### Supplementary Note 3

The behavior control system according to Supplementary Note 1, in which the robot is mounted on a storage container that stores clothes or controls a control target device mounted on a storage container that stores clothes.

### Supplementary Note 4

The behavior control system according to Supplementary Note 1, in which the robot is mounted on a stuffed toy or controls a control target device mounted on the stuffed toy.

### Other Embodiment 5

As another embodiment, the robot 100 may be applied to a robot mounted on a device such as an electrical appliance, a computer, an automobile, a motorcycle, or a toy, or the robot 100 may control a device such as an electrical appliance, a computer, an automobile, a motorcycle, or a toy. Furthermore, the robot 100 may be mounted on a stuffed toy, or may be applied to a control device connected to a control target device (speaker or camera) mounted on the stuffed toy in a wireless or wired manner. Other embodiments are specifically configured as follows. For example, the robot 100 may be applied to control the intercom.

The intercom controlled by the robot 100 includes, for example, a master unit installed in a room and a slave unit installed at, for example, a gate or an entrance. The master unit includes a response button, a monitor, a microphone, a speaker, and the like. The slave unit includes a call button, a camera, a microphone, a speaker, and the like. The master unit and the slave unit are connected to each other in a wireless or wired manner. Furthermore, the robot 100 and the master unit, and the robot 100 and the slave unit, are also connected to each other in a wireless or wired manner.

For example, when the call button of the slave unit is pressed by a visitor, the master unit notifies that a call has been made. At this time, the robot 100 may also notify that a call has been made by uttering, for example, “A VISITOR HAS ARRIVED”.

The robot 100 executes processing of determining the processing content of the intercom according to the preference of the user, the situation of the user, and the reaction of the user by Steps 1 to 5-2 below.

(Step 1) The robot 100 acquires the state of the user 10, the emotion value of the user 10, the emotion value of the robot 100, and the history data 222. Specifically, processing similar to above steps S100 to S103 is performed to acquire the state of the user 10, the emotion value of the user 10, the emotion value of the robot 100, and the history data 222.

(Step 2) The robot 100 acquires the preference of the user 10 regarding the intercom. Here, the “preference of the user 10 regarding the intercom” is information regarding the operation of the intercom.

For example, the information may be information regarding the operation setting of the intercom, such as the volume of the speaker of the master unit or the slave unit, the ringing tone of the intercom, the color and light amount of a light emitting diode (LED) indicator, and the brightness and contrast of the monitor.

Furthermore, the information may be information regarding response content to the user, such as an attitude when the robot 100 speaks to the user via the master unit (for example, wanting the robot to speak slowly in a cheerful tone), or the conversation content when the robot 100 speaks to the user via the master unit (for example, wanting the robot to be concerned about the physical condition of the user).

Furthermore, the information may be information regarding response content to a visitor, such as an attitude when the robot 100 speaks to a visitor via the slave unit (for example, wanting the robot to speak slowly in a cheerful tone to familiar visitors, and wanting the robot to speak rapidly in a polite tone to undesired visitors such as salespersons), or the conversation content when the robot 100 speaks to a visitor via the slave unit (for example, wanting the robot to greet familiar visitors, and wanting the robot to refuse undesired visitors such as salespersons).

Specifically, the behavior determination unit 236 determines, as a behavior of the robot 100, an utterance to the user 10 to ask a question about a preference regarding the intercom, and the behavior control unit 250 controls the control target 252 and makes an utterance to the user 10 to ask the question about the preference regarding the intercom. The user state recognition unit 230 recognizes the preference of the user 10 regarding the intercom based on the information (for example, the user's answer) analyzed by the sensor module unit 210.

(Step 3) The robot 100 determines the processing content of the intercom proposed to the user 10. The processing content of the intercom may be, for example, content of any item such as an item regarding operation setting of the intercom, an item related to response content to the user, and an item regarding response content to a visitor.

Hereinafter, as an example, a case where the robot 100 determines an attitude when responding to the user will be described. Here, the “attitude” is a feature consistently appearing in an utterance mode, a size of a gesture, and the like by the robot 100, and may be any item such as a bright attitude, a dark attitude, a lively attitude, and a calm attitude. Furthermore, a plurality of attitudes may be set in combination. With the content of this “attitude”, the robot 100 changes the wording in conversation, the emotion included in the voice, the magnitude of the voice, the speed of utterance, the magnitude of gesture, and the like.

Specifically, the behavior determination unit 236 adds a fixed sentence such as “AT THIS TIME, WHAT IS THE ATTITUDE RECOMMENDED TO THE USER?” to the text representing the preference of the user 10 regarding the attitude, the emotion of the user 10, the emotion of the robot 100, and the content stored in the history data 222, inputs the text into the sentence generation model, and acquires the recommended content regarding the attitude. At this time, it is possible to propose the attitude suitable for the user 10 by taking into consideration not only the preference of the user 10 regarding the attitude but also the emotion of the user 10 and the history data 222. Furthermore, by taking into consideration the emotion of the robot 100, it is possible to make the user 10 feel that the robot 100 has an emotion.

(Step 4) The robot 100 proposes the attitude determined in Step 3 to the user 10 and acquires the reaction of the user 10. Specifically, the behavior determination unit 236 determines, as a behavior of the robot 100, an utterance to the user 10 for proposing the attitude, and the behavior control unit 250 controls the control target 252 and makes an utterance to the user 10 for proposing the attitude. The utterance that proposes an attitude is, for example, an utterance such as “I WILL RESPOND CHEERFULLY AND ENERGETICALLY, IS THAT OKAY?”.

The user state recognition unit 230 recognizes the state of the user 10 based on the information analyzed by the sensor module unit 210, and the emotion determination unit 232 determines an emotion value indicating the emotion of the user 10 based on the information analyzed by the sensor module unit 210 and the state of the user 10 recognized by the user state recognition unit 230.

The behavior determination unit 236 determines whether the reaction of the user 10 is positive based on the state of the user 10 recognized by the user state recognition unit 230 and the emotion value indicating the emotion of the user 10, and determines whether to execute processing according to the attitude proposed to the user 10 or to propose another attitude to the user 10 as the behavior of the robot 100.

(Step 5-1) In a case where the reaction of the user 10 is positive, the robot 100 executes processing according to the proposed attitude. Specifically, in a case where it is determined that the processing according to the attitude proposed to the user 10 is executed as the behavior of the robot 100, the behavior control unit 250 controls the intercom as the control target 252 to execute the processing (for example, a conversation according to a cheerful and energetic attitude) according to the attitude proposed to the user 10.

(Step 5-2) In a case where the reaction of the user 10 is not positive, the robot 100 determines another attitude proposed to the user 10. Specifically, in a case where it is determined to propose another attitude to the user 10 as the behavior of the robot 100, the behavior determination unit 236 adds a fixed sentence such as “ARE THERE ANY OTHER ATTITUDES RECOMMENDED TO THE USER?” to the text representing the preference of the user 10 regarding the attitude, the emotion of the user 10, the emotion of the robot 100, and the content stored in the history data 222, inputs the text to a sentence generation model, and acquires the recommended content regarding the attitude. Then, the processing returns to Step 4 described above, and the processing of Steps 4 to 5-2 described above is repeated until it is determined to execute the processing according to the attitude proposed to the user 10.

In this regard, the robot 100 can execute processing of determining the processing content of the intercom according to the preference of the user, the situation of the user, and the reaction of the user.

With regard to the above embodiments, the following supplementary notes are further disclosed.

### Supplementary Note 1

A behavior control system including
a user state recognition unit that recognizes a user state including a behavior of a user,
an emotion determination unit that determines an emotion of the user or an emotion of a robot,
and a behavior determination unit that determines a behavior of the robot corresponding to the user state and the emotion of the user or the emotion of the robot based on a sentence generation model having an interaction function of causing the user to interact with the robot, in which
the behavior determination unit determines processing content of an intercom based on the emotion of the user or the emotion of the robot.

### Supplementary Note 2

The behavior control system according to Supplementary Note 1, in which
the behavior determination unit changes at least one of an operation setting of the intercom, a response content to the user, or a response content to a visitor based on the emotion of the user or the emotion of the robot.

### Supplementary Note 3

The behavior control system according to Supplementary Note 1 or 2, in which
the robot is mounted on a stuffed toy or is connected to a control target device mounted on the stuffed toy in a wireless or wired manner.

### Other Embodiment 6

As another embodiment, the robot 100 may be applied to a robot mounted on a device such as an electrical appliance, a computer, an automobile, a motorcycle, or a toy, or the robot 100 may control a device such as an electrical appliance, a computer, an automobile, a motorcycle, or a toy. Furthermore, the robot 100 may be mounted on a stuffed toy, or may be applied to a control device connected to a control target device (speaker or camera) mounted on the stuffed toy in a wireless or wired manner. Other embodiments are specifically configured as follows. For example, the robot 100 may be applied to mattress control.

The mattress controlled by the robot 100 includes a mattress main body, a temperature adjustment mechanism capable of adjusting the temperature of the mattress main body, and a hardness adjustment mechanism capable of adjusting the hardness of the mattress main body. The mattress main body is bedding on which a user lies down. As the mattress main body, various types such as a Bonnell coil mattress, a pocket coil mattress, a urethane mattress, and a latex mattress can be adopted. In addition, the mattress main body may be one on which one user lies down, or may be one on which a plurality of users lies down simultaneously.

An example of the temperature adjustment mechanism includes a sheet-shaped heater laid on a surface of the mattress main body. This heater is disposed over substantially the entire surface of the mattress main body, and the temperature can be individually adjusted (heated) for each of the plurality of portions. For example, in a case where the size of the mattress main body is such that two users can lie down on their side at the same time, the heater may be able to individually adjust the temperature on the left and right sides of the mattress main body.

In addition, the heater can individually adjust the temperature of the upper portion, the lower portion, and the central portion (that is, between the upper part and the lower part,) of the mattress main body regardless of the number of users lying down on the mattress main body. The upper portion of the mattress main body is, for example, a place where the head of a lying user is disposed. The lower portion of the mattress main body is, for example, a place where the legs of a lying user are disposed. The central portion of the mattress main body is, for example, a place where the waist or abdomen of a lying user is disposed.

As the temperature adjustment mechanism, for example, a cooling device such as a water-cooling type may be provided in addition to the heater. In addition, as the temperature adjustment mechanism, an air conditioner in a room in which the mattress main body is installed may be used. In this case, the mattress main body may be provided with an air chamber, and conditioned air may be introduced into the air chamber via a duct. The air-conditioned air introduced into the air chamber can heat or cool the mattress main body. Alternatively, an air outlet may be provided in the air chamber to discharge the conditioned air toward the user lying down on the mattress main body.

The hardness adjustment mechanism is used in combination with the mattress main body. The hardness adjustment mechanism is, for example, a bag-shaped sealed container having flexibility and an air moving device. The air moving device can supply air into the sealed container or can draw air from the sealed container.

The configuration of the hardness adjustment mechanism can be appropriately changed according to the form of the mattress main body. For example, in a case where the mattress main body is a Bonnell coil mattress or a pocket coil mattress, the coil is enclosed inside the sealed container. When air is supplied to the sealed container in this state, the internal pressure increases, and the hardness of the mattress main body can be increased. On the other hand, even when air is drawn from the sealed container, the coil is compressed, and the hardness of the mattress main body can be increased.

For example, when the mattress main body is a urethane mattress, urethane is enclosed inside the sealed container. When air is supplied to the sealed container in this state, the internal pressure increases and the hardness of the mattress main body increases. On the other hand, even when air is drawn from the sealed container, the mattress main body is compressed and the hardness increases. As described above, since the principle of increasing the hardness of the mattress main body varies depending on whether air is supplied or drawn, it is possible to provide an environment according to the preference of the lying user.

The robot 100 performs processing of adjusting the temperature and hardness of the mattress main body according to the preference of the user, the situation of the user, and the reaction of the user by Steps 1 to 5-2 below.

(Step 1) The robot 100 acquires the state of the user 10, the emotion value of the user 10, the emotion value of the robot 100, and the history data 222. Specifically, processing similar to above steps S100 to S103 is performed to acquire the state of the user 10, the emotion value of the user 10, the emotion value of the robot 100, and the history data 222.

(Step 2) The robot 100 acquires the preference of the user 10 regarding the temperature and the hardness of the mattress main body.

Specifically, the behavior determination unit 236 determines, as a behavior of the robot 100, an utterance to the user 10 to ask a question about a preference regarding the temperature and the hardness of the mattress main body, and the behavior control unit 250 controls the control target 252 and makes an utterance to the user 10 to ask the question about the preference regarding the temperature and the hardness of the mattress main body. The user state recognition unit 230 recognizes the preference of the user 10 regarding the temperature and the hardness of the mattress main body based on the information (for example, the user's answer) analyzed by the sensor module unit 210.

(Step 3) The robot 100 determines the temperature and the hardness of the mattress main body proposed to the user 10.

Specifically, the behavior determination unit 236 adds a fixed sentence such as “AT THIS TIME, WHAT ARE THE TEMPERATURE AND HARDNESS OF THE MATTRESS MAIN BODY RECOMMENDED TO THE USER?” to the text representing the preference of the user 10 regarding the temperature and the hardness of the mattress main body, the emotion of the user 10, the emotion of the robot 100, and the content stored in the history data 222, inputs the text to a sentence generation model, and acquires the recommended content regarding the temperature and the hardness of the mattress main body. At this time, it is possible to propose a sleep environment suitable for the user 10 by taking into consideration not only the preference regarding the temperature and hardness of the mattress main body of the user 10 but also the emotion of the user 10 and the history data 222. Furthermore, by taking into consideration the emotion of the robot 100, it is possible to make the user 10 feel that the robot 100 has an emotion.

(Step 4) The robot 100 proposes the temperature and the hardness of the mattress main body determined in Step 3 to the user 10, and acquires the reaction of the user 10.

Specifically, the behavior determination unit 236 determines, as a behavior of the robot 100, an utterance to the user 10 for proposing the temperature and the hardness of the mattress main body, and the behavior control unit 250 controls the control target 252 and makes an utterance to the user 10 for proposing the temperature and the hardness of the mattress main body. The user state recognition unit 230 recognizes the state of the user 10 based on the information analyzed by the sensor module unit 210, and the emotion determination unit 232 determines an emotion value indicating the emotion of the user 10 based on the information analyzed by the sensor module unit 210 and the state of the user 10 recognized by the user state recognition unit 230.

The behavior determination unit 236 determines whether the reaction of the user 10 is positive based on the state of the user 10 recognized by the user state recognition unit 230 and the emotion value indicating the emotion of the user 10, and determines whether to execute processing of adjusting the temperature and the hardness of the mattress main body proposed to the user 10 or to propose another temperature and hardness to the user 10 as the behavior of the robot 100.

(Step 5-1) In a case where the reaction of the user 10 is positive, the robot 100 executes processing of adjusting the temperature and the hardness of the mattress main body to the proposed temperature and hardness.

Specifically, in a case where it is determined to execute processing of adjusting the temperature and the hardness of the mattress main body to the temperature and the hardness proposed to the user 10 as the behavior of the robot 100, the behavior control unit 250 controls the temperature adjustment mechanism and the hardness adjustment mechanism as the control target 252 to adjust the temperature and the hardness of the mattress main body to the temperature and the hardness proposed to the user 10.

(Step 5-2) In a case where the reaction of the user 10 is not positive, the robot 100 determines another temperature and hardness to be proposed to the user 10.

Specifically, in a case where it is determined to propose another temperature and hardness to the user 10 as the behavior of the robot 100, the behavior determination unit 236 adds a fixed sentence such as “ARE THERE ANY OTHER TEMPERATURE AND HARDNESS OF THE MATTRESS MAIN BODY RECOMMENDED TO THE USER?” to the text representing the preference of the user 10 regarding the temperature and the hardness of the mattress main body, the emotion of the user 10, the emotion of the robot 100, and the content stored in the history data 222, inputs the text to a sentence generation model, and acquires the recommended content regarding the temperature and the hardness of the mattress main body.

Then, the processing returns to Step 4, and the processing of Steps 4 to 5-2 is repeated until it is determined to execute processing of adjusting the temperature and the hardness of the mattress main body to the temperature and the hardness proposed to the user 10.

In this regard, the robot 100 can execute processing of adjusting the temperature and the hardness of the mattress main body according to the preference of the user, the situation of the user, and the reaction of the user.

Note that the robot 100 may include a sensor that detects the sleep state of the user as the user state. As the sensor, a sensor that detects whether the user is sleeping from the motion of the body of the user, a sensor that detects whether the user is sleeping from the heart rate, pulse, or the like of the user, or the like can be used. Such a sensor can also be provided in a wearable terminal worn by the user. When the sensor of the robot 100 detects that the user has fallen asleep, the robot 100 regards that a temperature and a hardness of a mattress main body are in a comfortable state for the user, and maintains the temperature and the hardness.

Alternatively, the temperature and the hardness of the mattress main body may be changed according to the depth of sleep.

The mattress may include any one of the temperature adjustment mechanism and the hardness adjustment mechanism instead of both the temperature adjustment mechanism and the hardness adjustment mechanism. In this case, in the above step, the robot 100 adjusts any one of the temperature and the hardness of the mattress main body.

With regard to the above embodiments, the following supplementary notes are further disclosed.

### Supplementary Note 1

A behavior control system including
a user state recognition unit that recognizes a user state including a behavior of a user,
an emotion determination unit that determines an emotion of the user or an emotion of a robot,
and a behavior determination unit that determines a behavior of the robot corresponding to the user state and the emotion of the user or the emotion of the robot based on a sentence generation model having an interaction function of causing the user to interact with the robot, in which
the behavior determination unit is capable of adjusting at least one of a temperature or elasticity of a mattress.

### Supplementary Note 2

The behavior control system according to Supplementary Note 1, in which the behavior determination unit is capable of individually adjusting a temperature for each part of the mattress.

### Supplementary Note 3

The behavior control system according to Supplementary Note 1 or 2, in which the behavior determination unit is capable of individually adjusting elasticity for each part of the mattress.

### Supplementary Note 4

The behavior control system according to Supplementary Note 1, in which the behavior determination unit holds the temperature and elasticity according to the user state.

### Supplementary Note 5

The behavior control system according to Supplementary Note 1, in which the robot is mounted on a stuffed toy or is connected to a control target device mounted on the stuffed toy in a wireless or wired manner.

### Other Embodiment 7

As another embodiment, the robot 100 may be applied to a robot mounted on a device such as an electrical appliance, a computer, an automobile, a motorcycle, or a toy, or the robot 100 may control a device such as an electrical appliance, a computer, an automobile, a motorcycle, or a toy. Furthermore, the robot 100 may be mounted on a stuffed toy, or may be applied to a control device connected to a control target device (speaker or camera) mounted on the stuffed toy in a wireless or wired manner. Furthermore, the device in which the robot 100 is mounted may be mounted on a stuffed toy, or the robot 100 may be connected to the control device connected to a control target device (speaker or camera) mounted on the stuffed toy in a wireless or wired manner. Other embodiments are specifically configured as follows. For example, the robot 100 may be applied to a massage chair.

The robot 100 as a massage chair includes a chair main body having a reclining function for holding the body of the user, a massage mechanism for massaging various parts such as a shoulder, a neck, a back, a waist, legs, and arms of the user held by the chair main body, and the like.

The robot 100 executes processing of changing the setting of at least one of the kneading intensity of the massage chair, the massage course, the massage time, or the massage part according to the preference of the user, the situation of the user, and the reaction of the user by Steps 1 to 5-2 below.

(Step 1) The robot 100 acquires the state of the user 10, the emotion value of the user 10, the emotion value of the robot 100, and the history data 222. Specifically, processing similar to above steps S100 to S103 is performed to acquire the state of the user 10, the emotion value of the user 10, the emotion value of the robot 100, and the history data 222.

(Step 2) The robot 100 acquires the preference of the user 10 regarding a massage.

Specifically, the behavior determination unit 236 determines, as a behavior of the robot 100, an utterance to the user 10 to ask a question about a preference regarding the massage, and the behavior control unit 250 controls the control target 252 and makes an utterance to the user 10 to ask the question about the preference regarding the massage. The user state recognition unit 230 recognizes the preference of the user 10 regarding the massage based on the information (for example, the user's answer) analyzed by the sensor module unit 210. For example, the robot 100 utters a question to the user 10, such as “WHICH PART OF YOUR BODY FEELS STIFF?” or “DO YOU PREFER A STRONGER MASSAGE, OR A WEAKER ONE?” Then, the robot 100 recognizes the preference of the user 10 regarding massage based on the answer such as “MY SHOULDERS FEEL STIFF TODAY.” or “MY CALVES ARE TIRED TODAY.” from the user 10.

(Step 3) The robot 100 determines the kneading intensity (strong or weak) of the massage chair, the massage time, the massage part, and the massage course (hereinafter referred to as a massage course or the like.) proposed to the user 10.

Specifically, the behavior determination unit 236 adds a fixed sentence such as “AT THIS TIME, WHAT IS THE MASSAGE RECOMMENDED TO THE USER?” to the text representing the preference of the user 10 regarding the massage, the emotion of the user 10, the emotion of the robot 100, and the content stored in the history data 222, inputs the text into the sentence generation model, and acquires the recommended content regarding the massage. At this time, it is possible to propose a massage course or the like suitable for the emotion of the user 10 at that time by taking into consideration not only the preference of the user 10 regarding the massage but also the emotion of the user 10 and the history data 222. Furthermore, by taking into consideration the emotion of the robot 100, it is possible to make the user 10 feel that the robot 100 has an emotion. For example, even if the desire of the massage of the user 10 is the shoulder massage course, in a case where it is determined that it is better to energize the user 10, the robot 100 performs control to propose addition of the whole-body course.

(Step 4) The robot 100 proposes the massage course or the like determined in Step 3 to the user 10, and acquires the reaction of the user 10.

Specifically, the behavior determination unit 236 determines, as a behavior of the robot 100, an utterance to the user 10 for proposing the selected massage course or the like, and the behavior control unit 250 controls the control target 252 and makes an utterance to the user 10 for proposing the selected massage course or the like.

The user state recognition unit 230 recognizes the state of the user 10 based on the information analyzed by the sensor module unit 210, and the emotion determination unit 232 determines an emotion value indicating the emotion of the user 10 based on the information analyzed by the sensor module unit 210 and the state of the user 10 recognized by the user state recognition unit 230.

The behavior determination unit 236 determines whether the reaction of the user 10 is positive based on the state of the user 10 recognized by the user state recognition unit 230 and the emotion value indicating the emotion of the user 10, and determines whether to confirm the massage course or the like proposed to the user 10 or to propose another massage course or the like to the user 10 as the behavior of the robot 100.

(Step 5-1) In a case where the reaction of the user 10 is positive, the robot 100 executes processing for causing the massage chair to execute the proposed massage course.

Specifically, in a case where it is determined that the massage course or the like proposed to the user 10 is executed as the behavior of the robot 100, the behavior control unit 250 controls the massage chair which is the control target 252 such that the massage course or the like proposed to the user 10 is executed.

(Step 5-2) In a case where the reaction of the user 10 is not positive, the robot 100 determines another massage course or the like to be proposed to the user 10.

Specifically, in a case where it is determined to propose another massage course or the like to the user 10 as the behavior of the robot 100, the behavior determination unit 236 adds a fixed sentence such as “ARE THERE ANY OTHER MASSAGES RECOMMENDED TO THE USER?” to the text representing the preference of the user 10 regarding the massage, the emotion of the user 10, the emotion of the robot 100, and the content stored in the history data 222, inputs the text to a sentence generation model, and acquires the recommended content regarding the massage. Then, the processing returns to Step 4 described above, and the processing of Steps 4 to 5-2 described above is repeated until it is determined to execute the massage course or the like proposed to the user 10.

In this regard, the robot 100 can execute processing of selecting the massage course or the like to be executed in the massage chair according to the preference of the user, the situation of the user, and the reaction of the user, and controlling the massage chair such that the selected massage course or the like is executed.

Although the case where the behavior determination unit 236 changes the massage course or the like in the massage chair based on the emotion of the user 10 or the emotion of the robot 100 has been described, various settings regarding the massage may be changed in addition to the massage course or the like. That is, the behavior determination unit 236 may control the massage chair by changing various settings such as the kneading intensity (strong or weak) of the massage chair, the massage time, and the massage part based on the emotion of the user 10 or the emotion of the robot 100.

With regard to the above embodiments, the following supplementary notes are further disclosed.

### Supplementary Note 1

A behavior control system including
a user state recognition unit that recognizes a user state including a behavior of a user,
an emotion determination unit that determines an emotion of the user or an emotion of a robot,
and a behavior determination unit that determines a behavior of the robot corresponding to the user state and the emotion of the user or the emotion of the robot based on an interaction function of causing the user to interact with the robot, in which
the robot is mounted on a massage chair, and
the behavior determination unit changes at least one setting among kneading strength of the massage chair, massage course, massage time, and massage part based on the emotion of the user or the emotion of the robot.

### Supplementary Note 2

The behavior control system according to Supplementary Note 1, in which the massage chair is connected to a control target device mounted on the stuffed toy in a wireless or wired manner.

### Other Embodiment 8

As another embodiment, the robot 100 may be applied to a robot mounted on a device such as an electrical appliance, a computer, an automobile, a motorcycle, or a toy, or the robot 100 may control a device such as an electrical appliance, a computer, an automobile, a motorcycle, or a toy. Furthermore, the robot 100 may be mounted on a stuffed toy, or may be applied to a control device connected to a control target device (speaker or camera) mounted on the stuffed toy in a wireless or wired manner. Other embodiments are specifically configured as follows. For example, the robot 100 may be applied to control of a head-mounted display.

The head-mounted display controlled by the robot 100 includes a display, a speaker, a content reproduction unit, and the like. The robot 100 and the head-mounted display are connected to each in a wireless or wired manner.

The robot 100 executes processing of determining the processing content of the head-mounted display according to the preference of the user, the situation of the user, and the reaction of the user by Steps 1 to 5-2 below.

(Step 1) The robot 100 acquires the state of the user 10, the emotion value of the user 10, the emotion value of the robot 100, and the history data 222. Specifically, processing similar to above steps S100 to S103 is performed to acquire the state of the user 10, the emotion value of the user 10, the emotion value of the robot 100, and the history data 222.

(Step 2) The robot 100 acquires a preference of the user 10 regarding the head-mounted display. Here, the “preference of the user 10 regarding the head-mounted display” is, for example, information regarding image quality, sound quality, content to be reproduced on the head-mounted display, and the like.

Specifically, the behavior determination unit 236 determines, as a behavior of the robot 100, an utterance to the user 10 to ask a question about a preference regarding the head-mounted display, and the behavior control unit 250 controls the control target 252, and makes an utterance to the user 10 to ask the question about the preference regarding the head-mounted display. The user state recognition unit 230 recognizes the preference of the user 10 regarding the head-mounted display based on the information (for example, the user's answer) analyzed by the sensor module unit 210.

(Step 3) The robot 100 determines the processing content of the head-mounted display proposed to the user 10. The processing content of the head-mounted display is, for example, processing of changing at least one of the operation time, the image quality, and the sound quality of the head-mounted display, or the content to be reproduced on the head-mounted display.

Here, the operation time of the head-mounted display is a time until the power of the head-mounted display is forcibly turned off. For example, the operation time may be a time from when the power of the head-mounted display is turned on until when the power is turned off, or may be a time from when the continuous use time of the head-mounted display by the user exceeds a set threshold value until when the power is turned off.

In addition, the image quality of the head-mounted display is, for example, characteristics such as luminance and contrast of the display. Furthermore, the sound quality of the head-mounted display is, for example, characteristics such as volume and frequency characteristics of reproduced sound.

In the following description, as an example, it is assumed that the robot 100 determines the image quality of the head-mounted display.

Specifically, the behavior determination unit 236 adds a fixed sentence such as “AT THIS TIME, WHAT IS THE IMAGE QUALITY RECOMMENDED TO THE USER?” to the text representing the preference of the user 10 regarding the image quality, the emotion of the user 10, the emotion of the robot 100, and the content stored in the history data 222, inputs the text into the sentence generation model, and acquires the recommended content regarding the image quality. At this time, it is possible to propose the image quality suitable for the user 10 by taking into consideration not only the preference of the user 10 regarding the image quality but also the emotion of the user 10 and the history data 222. Furthermore, by taking into consideration the emotion of the robot 100, it is possible to make the user 10 feel that the robot 100 has an emotion.

(Step 4) The robot 100 proposes the image quality determined in Step 3 to the user 10 and acquires the reaction of the user 10. Specifically, the behavior determination unit 236 determines, as a behavior of the robot 100, an utterance to the user 10 for proposing an image quality, and the behavior control unit 250 controls the control target 252 and makes an utterance to the user 10 for proposing the image quality. For the proposal of the image quality, after the image quality of the head-mounted display is changed to the proposed image quality, for example, an utterance such as “IS CURRENT BRIGHTNESS AND CONTRAST ACCEPTABLE?” may be made.

The user state recognition unit 230 recognizes the state of the user 10 based on the information analyzed by the sensor module unit 210, and the emotion determination unit 232 determines an emotion value indicating the emotion of the user 10 based on the information analyzed by the sensor module unit 210 and the state of the user 10 recognized by the user state recognition unit 230.

The behavior determination unit 236 determines whether the reaction of the user 10 is positive based on the state of the user 10 recognized by the user state recognition unit 230 and the emotion value indicating the emotion of the user 10, and determines whether to execute processing of the setting the image quality proposed to the user 10 or to propose another image quality to the user 10 as the behavior of the robot 100.

(Step 5-1) In a case where the reaction of the user 10 is positive, the robot 100 executes processing of setting the proposed image quality. Specifically, in a case where it is determined that the processing of setting the image quality proposed to the user 10 is executed as the behavior of the robot 100, the behavior control unit 250 controls the head-mounted display as the control target 252 to execute the processing of setting the image quality proposed to the user 10.

(Step 5-2) In a case where the reaction of the user 10 is not positive, the robot 100 determines another image quality to be proposed to the user 10. Specifically, in a case where it is determined to propose another image quality to the user 10 as the behavior of the robot 100, the behavior determination unit 236 adds a fixed sentence such as “ARE THERE ANY OTHER IMAGE QUALITIES RECOMMENDED TO THE USER?” to the text representing the preference of the user 10 regarding the image quality, the emotion of the user 10, the emotion of the robot 100, and the content stored in the history data 222, inputs the text to a sentence generation model, and acquires the recommended content regarding the image quality. Then, the processing returns to Step 4 described above, and the processing of Steps 4 to 5-2 described above is repeated until it is determined to execute the processing of setting the image quality proposed to the user 10.

In this regard, the robot 100 can execute processing of determining the processing content of the head-mounted display according to the preference of the user, the situation of the user, and the reaction of the user.

Furthermore, in addition to the above processing, in a case where a continuous use time of the head-mounted display by the user exceeds a set threshold value, the robot 100 may change at least one of the operation time, the image quality, and the sound quality of the head-mounted display, or the content to be reproduced on the head-mounted display to content more favorable to the health condition of the user as compared with before the threshold value was exceeded.

Here, “changing to content favorable to the health condition of the user” refers, for example, in the case of the operating time of the head-mounted display, to processing of setting the operation time to a shorter time than the current setting value. In addition, in the case of the image quality of the head-mounted display, it is processing of setting the luminance and/or the contrast to a value lower than the current setting value. In addition, in the case of the sound quality of the head-mounted display, it is processing of setting the volume to a value lower than the current setting value and/or setting the frequency characteristic to a value lower in stimulation than the current setting value (for example, a value obtained by lowering the volume in the low frequency region and the high frequency region). Furthermore, in the case of content to be reproduced on the head-mounted display, for example, it is processing of changing to content with less stimulation such as healing music video (MV).

For example, in a case where the continuous use time of the head-mounted display by the user exceeds the threshold value of two hours, the fatigue of the eyes and ears of the user can be reduced by lowering the luminance and the volume of the head-mounted display. Furthermore, in a case where the continuous use time of the head-mounted display by the user exceeds the threshold value of two hours, the content to be reproduced on the head-mounted display is changed to the healing MV, thereby reducing the fatigue of the brain of the user. In this regard, the robot 100 can also manage the health condition of the user who uses the head-mounted display.

With regard to the above embodiments, the following supplementary notes are further disclosed.

### Supplementary Note 1

A behavior control system including
a user state recognition unit that recognizes a user state including a behavior of a user,
an emotion determination unit that determines an emotion of the user or an emotion of a robot,
and a behavior determination unit that determines a behavior of the robot corresponding to the user state and the emotion of the user or the emotion of the robot based on a sentence generation model having an interaction function of causing the user to interact with the robot, in which
the behavior determination unit determines processing content of a head-mounted display based on the emotion of the user or the emotion of the robot.

### Supplementary Note 2

The behavior control system according to Supplementary Note 1, in which
the behavior determination unit changes at least one of an operation time, image quality, or sound quality of the head-mounted display, and content to be reproduced on the head-mounted display based on the emotion of the user or the emotion of the robot.

### Supplementary Note 3

The behavior control system according to Supplementary Note 1, in which
in a case where a continuous use time of the head-mounted display by the user exceeds a set threshold value, the behavior determination unit changes at least one of the operation time, the image quality, and the sound quality of the head-mounted display, or the content to be reproduced on the head-mounted display to content more favorable to the health condition of the user as compared with before the threshold value was exceeded.

### Supplementary Note 4

The behavior control system according to any one of Supplementary Notes 1 to 3, in which
the robot is mounted on a stuffed toy or is connected to a control target device mounted on the stuffed toy in a wireless or wired manner.

### Other Embodiment 9

As another embodiment, the robot 100 may be applied to a robot mounted on a device such as an electrical appliance, a computer, an automobile, a motorcycle, or a toy, or the robot 100 may control a device such as an electrical appliance, a computer, an automobile, a motorcycle, or a toy. Furthermore, the robot 100 may be mounted on a stuffed toy, or may be applied to a control device connected to a control target device (speaker or camera) mounted on the stuffed toy in a wireless or wired manner. Other embodiments are specifically configured as follows. For example, the robot 100 may be applied to control of an AI bath device.

The AI bath device controlled by the robot 100 includes, for example, a water supply unit, a heat source unit, and an air conditioner. The heat source unit includes a water temperature sensor and a water amount sensor. The air conditioner adjusts the temperature and humidity in the bathroom. An air conditioner for adjusting the temperature and humidity outside the bathroom, for example, in the dressing room may be further provided.

The robot 100 executes processing of setting parameters in the AI bath device according to the preference of the user, the situation of the user, and the reaction of the user by Steps 1 to 5-2 below.

(Step 1) The robot 100 acquires the state of the user 10, the emotion value of the user 10, the emotion value of the robot 100, and the history data 222. Specifically, processing similar to above steps S100 to S103 is performed to acquire the state of the user 10, the emotion value of the user 10, the emotion value of the robot 100, and the history data 222.

(Step 2) The robot 100 acquires the preference of the user 10 regarding parameters such as the water temperature and the water amount of the bathtub, and the temperature and the humidity of the bathroom.

Specifically, the behavior determination unit 236 determines, as a behavior of the robot 100, an utterance to the user 10 to ask a question about a preference regarding parameters such as the water temperature and water amount of the bathtub and the temperature and humidity of the bathroom, and the behavior control unit 250 controls the control target 252 and makes an utterance to the user 10 to ask the question about the preference regarding parameters such as the water temperature and water amount of the bathtub and the temperature and humidity of the bathroom. The user state recognition unit 230 recognizes the preference of the user 10 regarding parameters such as the water temperature and the water amount of the bathtub, and the temperature and the humidity of the bathroom based on the information (for example, the user's answer) analyzed by the sensor module unit 210.

(Step 3) The robot 100 determines parameters to be proposed to the user 10.

Specifically, the behavior determination unit 236 adds, for example, a fixed sentence such as “AT THIS TIME, WHAT ARE THE WATER TEMPERATURE AND WATER AMOUNT OF THE BATHTUB AND THE TEMPERATURE AND HUMIDITY OF THE BATHROOM RECOMMENDED TO THE USER?” to the text representing the preference of the user 10 regarding parameters such as the water temperature and water amount of the bathtub and the temperature and the humidity of the bathroom, the emotion of the user 10, the emotion of the robot 100, and the content stored in the history data 222, inputs the text into the sentence generation model, and acquires the recommended content regarding parameters such as the water temperature and water amount of the bathtub and the temperature and the humidity of the bathroom. At this time, it is possible to propose parameters suitable for the user 10 by taking into consideration not only the preference of the user 10 regarding the parameters such as the water temperature and water amount of the bathtub and the temperature and the humidity of the bathroom but also the emotion of the user 10 and the history data 222. Furthermore, by taking into consideration the emotion of the robot 100, it is possible to make the user 10 feel that the robot 100 has an emotion.

(Step 4) The robot 100 proposes the parameters determined in Step 3 to the user 10 and acquires the reaction of the user 10.

Specifically, the behavior determination unit 236 determines, as a behavior of the robot 100, an utterance to the user 10 for proposing parameters, and the behavior control unit 250 controls the control target 252 and makes an utterance to the user 10 for proposing the parameters. The user state recognition unit 230 recognizes the state of the user 10 based on the information analyzed by the sensor module unit 210, and the emotion determination unit 232 determines an emotion value indicating the emotion of the user 10 based on the information analyzed by the sensor module unit 210 and the state of the user 10 recognized by the user state recognition unit 230.

The behavior determination unit 236 determines whether the reaction of the user 10 is positive based on the state of the user 10 recognized by the user state recognition unit 230 and the emotion value indicating the emotion of the user 10, and determines whether to execute processing of setting the parameters proposed to the user 10 in the AI bath device, or to propose other parameters to the user 10 as the behavior of the robot 100.

(Step 5-1) In a case where the reaction of the user 10 is positive, the robot 100 executes processing of setting the proposed parameters in the AI bath device.

Specifically, in a case where it is determined to execute processing of setting the parameters proposed to the user 10 in the AI bath device as the behavior of the robot 100, the behavior control unit 250 controls, for example, the water supply unit, the heat source unit, and the air conditioner, which are the control target 252, to execute the parameters proposed to the user 10.

(Step 5-2) In a case where the reaction of the user 10 is not positive, the robot 100 determines other parameters to be proposed to the user 10.

Specifically, in a case where it is determined to propose other parameters to the user 10 as the behavior of the robot 100, the behavior determination unit 236 adds, for example, a fixed sentence such as “ARE THERE OTHER WATER TEMPERATURE AND WATER AMOUNT OF THE BATHTUB, AND TEMPERATURE AND HUMIDITY OF THE BATHROOM RECOMMENDED TO THE USER?” to text representing a preference of the user 10 regarding parameters such as the water temperature and the water amount of the bathtub, the temperature and the humidity of the bathroom, the emotion of the user 10, the emotion of the robot 100, and the content stored in the history data 222, inputs the text to a sentence generation model, and acquires the recommended content regarding the parameters such as the water temperature and the water amount of the bathtub, or the temperature and the humidity of the bathroom. Then, the processing returns to Step 4 described above, and the processing of Steps 4 to 5-2 described above is repeated until it is determined to execute the processing of setting the parameters proposed to the user 10 in the AI bath device.

In this regard, the robot 100 can execute processing of setting the parameters in the AI bath device according to the preference of the user, the situation of the user, and the reaction of the user.

With regard to the above embodiments, the following supplementary notes are further disclosed.

### Supplementary Note 1

A behavior control system including
a user state recognition unit that recognizes a user state including a behavior of a user,
an emotion determination unit that determines an emotion of the user or an emotion of a robot,
and a behavior determination unit that determines a behavior of the robot corresponding to the user state and the emotion of the user or the emotion of the robot based on a sentence generation model having an interaction function of causing the user to interact with the robot, in which
the behavior determination unit proposes a parameter to be set to an AI bath device to the user.

### Supplementary Note 2

The behavior control system according to Supplementary Note 1, in which the robot is mounted on a stuffed toy or is connected to a control target device mounted on the stuffed toy in a wireless or wired manner.

### Other Embodiment 10

As another embodiment, the robot 100 may be applied to a robot mounted on a device such as an electrical appliance, a computer, an automobile, a motorcycle, or a toy, or the robot 100 may control a device such as an electrical appliance, a computer, an automobile, a motorcycle, or a toy. Furthermore, the robot 100 may be mounted on a stuffed toy, or may be applied to a control device connected to a control target device (speaker or camera) mounted on the stuffed toy in a wireless or wired manner. Other embodiments are specifically configured as follows. For example, the robot 100 may be mounted on fitness equipment. Furthermore, the robot 100 may be applied to control of the fitness equipment.

The fitness equipment on which the robot 100 is mounted is, for example, equipment that does not include a load-variable mechanism. Examples of the equipment not provided with the load-variable mechanism include equipment such as a training bench, a dumbbell, a barbell, or a sandbag, which can be used as an abdominal muscle stand or the like.

Further, the robot 100 may be mounted on fitness equipment including a load-variable mechanism. Examples of the equipment including the load-variable mechanism include equipment such as a chest press machine, a shoulder press machine, a lat pulldown machine, and a leg curl machine. The load-variable mechanism includes a plurality of weights lifted by the motion of the user and a conversion tool that changes the number of weights lifted by the motion of the user. The weights are stacked, for example, in the vertical direction, and an insertion hole into which a pin as a conversion tool can be inserted is formed in each of the weights. The training load applied to the user can be changed depending on which weight insertion hole the pin is inserted into. For example, if a pin is inserted into the insertion hole of the weight below in the stacking direction, the load can be increased. On the other hand, if the pin is inserted into the insertion hole of the weight above in the stacking direction, the load can be reduced.

The robot 100 mounted on these pieces of equipment makes an utterance, as encouragement, of the content corresponding to a user state (such as conversation preference, facial expression, heart rate, exercise speed, and the like to be described later) of the user who uses the fitness equipment. Furthermore, the robot 100 mounted on the fitness equipment provided with a load-variable mechanism can also change the training load applied to the user by controlling the load-variable mechanism of the machine (for example, by moving a pin).

The robot 100 executes processing of encouraging the user who is training by using the fitness equipment according to the preference of the user, the situation of the user, and the reaction of the user by Steps 1 to 5-2 below.

(Step 1) The robot 100 acquires the state of the user 10, the emotion value of the user 10, the emotion value of the robot 100, and the history data 222. Specifically, processing similar to above steps S100 to S103 is performed to acquire the state of the user 10, the emotion value of the user 10, the emotion value of the robot 100, and the history data 222.

(Step 2) The robot 100 acquires the preference of the user 10 regarding encouragement during the training. The encouragement is an utterance content of the robot 100 during training in which the user uses the fitness equipment.

Specifically, the behavior determination unit 236 determines, as a behavior of the robot 100, an utterance of asking the user 10 a question about a preference regarding the encouragement, and the behavior control unit 250 controls the control target 252 and makes an utterance for asking the user 10 a question about a preference (conversation preference) regarding the encouragement (for example, which is preferable between being praised with “GREAT!” and being encouraged with “YOU CAN DO MORE!”, or the like). The user state recognition unit 230 recognizes the preference of the user 10 regarding the encouragement based on the information (for example, the user's answer) analyzed by the sensor module unit 210.

(Step 3) The robot 100 determines the content to encourage the user 10.

Specifically, the behavior determination unit 236 adds a fixed sentence such as “AT THIS TIME, WHAT IS THE ENCOURAGEMENT RECOMMENDED TO THE USER?” to the text representing the preference of the user 10 regarding the encouragement, the emotion of the user 10, the emotion of the robot 100, and the content stored in the history data 222, inputs the text into the sentence generation model, and acquires the recommended content regarding the encouragement. At this time, it is possible to provide the encouragement suitable for the user 10 by taking into consideration not only the preference of the user 10 regarding the encouragement but also the emotion of the user 10 and the history data 222. Furthermore, by taking into consideration the emotion of the robot 100, it is possible to make the user 10 feel that the robot 100 has an emotion.

(Step 4) The robot 100 utters the encouragement determined in Step 3 to the user 10 during training, and acquires the reaction of the user 10.

Specifically, the behavior determination unit 236 determines, as a behavior of the robot 100, an utterance for encouraging the user 10 during training, and the behavior control unit 250 controls the control target 252 and makes an utterance for encouraging the user 10. The user state recognition unit 230 recognizes the state of the user 10 based on the information analyzed by the sensor module unit 210, and the emotion determination unit 232 determines an emotion value indicating the emotion of the user 10 based on the information analyzed by the sensor module unit 210 and the state of the user 10 recognized by the user state recognition unit 230. The behavior determination unit 236 determines whether the reaction of the user 10 is positive based on the state of the user 10 (such as facial expression, heart rate, or exercise speed) recognized by the user state recognition unit 230 and an emotion value indicating the emotion of the user 10, and determines, as a behavior of the robot 100, whether to execute processing of continuing the uttered encouragement to the user 10 or to make another utterance of encouragement to the user 10.

(Step 5-1) In a case where the reaction of the user 10 is positive, the robot 100 executes processing of continuing the uttered encouragement.

Specifically, in a case where it is determined to execute processing of continuing the uttered encouragement to the user 10 as the behavior of the robot 100, the behavior control unit 250 controls the robot 100 to continue the uttered encouragement to the user 10.

(Step 5-2) In a case where the reaction of the user 10 is not positive, the robot 100 determines another encouragement to utter to the user 10.

Specifically, in a case where it is determined to utter another encouragement to the user 10 as the behavior of the robot 100, the behavior determination unit 236 adds a fixed sentence such as “ARE THERE ANY OTHER ENCOURAGEMENTS RECOMMENDED TO THE USER?” to the text representing the preference of the user 10 regarding the encouragement, the emotion of the user 10, the emotion of the robot 100, and the content stored in the history data 222, inputs the text to a sentence generation model, and acquires the recommended content regarding the encouragement. Then, the processing returns to Step 4 described above, and the processing of Steps 4 to 5-2 described above is repeated until it is determined to execute the processing of continuing the encouragement proposed to the user 10.

The case where the reaction (facial expression) of the user 10 is positive is, for example, a case where the facial expression during the training interval is a smile. On the other hand, a case where the reaction is negative is, for example, a case where the facial expression during the training interval is angry.

Furthermore, the case where the reaction (heart rate) of the user 10 is positive is, for example, a case where the heart rate is a predetermined value or less. If the heart rate of the user 10 is equal to or less than the predetermined value, the robot 100 continues the encouragement and makes the user 10 continue the training. On the other hand, a case where the reaction is the negative is, for example, a case where the heart rate is greater than a predetermined value. If the heart rate of the user 10 is greater than a predetermined value, the robot 100 changes the encouragement content and stops the training.

Furthermore, the case where the reaction (exercise speed) of the user 10 is positive is, for example, a case where the exercise speed has increased. In a case where the exercise speed increases, the robot 100 continues the encouragement and makes the user 10 maintain the training speed. On the other hand, a case where the reaction is negative is, for example, a case where the exercise speed becomes slow. In a case where the exercise speed becomes slow, the robot 100 changes the encouragement content and makes the user 10 increase the training speed.

In this regard, the robot 100 can execute processing of encouraging according to the preference of the user, the situation of the user, and the reaction of the user.

With regard to the above embodiments, the following supplementary notes are further disclosed.

### Supplementary Note 1

A behavior control system including
a user state recognition unit that recognizes a user state including a behavior of a user, an emotion determination unit that determines an emotion of the user or an emotion of a robot,
and a behavior determination unit that determines a behavior of the robot corresponding to the user state and the emotion of the user or the emotion of the robot based on a sentence generation model having an interaction function of causing the user to interact with the robot, in which
the behavior determination unit determines a behavior of uttering content corresponding to the user state of the user who uses fitness equipment.

### Supplementary Note 2

The behavior control system according to Supplementary Note 1, in which the user state recognition unit recognizes a conversation preference of the user, as the user state, and
the behavior determination unit determines a behavior of uttering content according to the conversation preference of the user.

### Supplementary Note 3

The behavior control system according to Supplementary Note 1, in which the user state recognition unit recognizes a facial expression of the user as the user state, and
the behavior determination unit determines a behavior of uttering content according to the facial expression of the user.

### Supplementary Note 4

The behavior control system according to Supplementary Note 1, in which the user state recognition unit recognizes a heart rate of the user as the user state, and
the behavior determination unit determines a behavior of uttering content according to the heart rate of the user.

### Supplementary Note 5

The behavior control system according to Supplementary Note 1, in which the user state recognition unit recognizes an exercise speed of the user as the user state, and
the behavior determination unit determines a behavior of uttering content according to the exercise speed of the user.

### Supplementary Note 6

The behavior control system according to Supplementary Note 1, in which the robot is mounted on a stuffed toy or is connected to a control target device mounted on the stuffed toy in a wireless or wired manner.

### Other Embodiment 11

As another embodiment, the robot 100 may be applied to a robot mounted on a device such as an electrical appliance, a computer, an automobile, a motorcycle, or a toy, or the robot 100 may control a device such as an electrical appliance, a computer, an automobile, a motorcycle, or a toy. Furthermore, the robot 100 may be mounted on a stuffed toy, or may be applied to a control device connected to a control target device (speaker or camera) mounted on the stuffed toy in a wireless or wired manner. Other embodiments are specifically configured as follows. For example, the robot 100 may also be applied to control of a motorcycle (also referred to as a motorized two-wheeled vehicle).

The motorcycle, on which the robot 100 is mounted or which is connected to the robot 100 in such a manner as to be controllable by the robot 100, includes, for example, front and rear wheels, a prime mover including an internal combustion engine or a motor and driving at least a rear wheel, front and rear suspensions, a suspension adjustment unit provided to adjust a hardness of the suspensions (a spring preload, a compression-side damping force of a damper, and a rebound-side damping force of the damper) by a first actuator, a handlebar adjustment unit provided to adjust a height of handlebars by a second actuator, and a seat height adjustment unit provided to adjust a height of a seat by a third actuator. The robot 100 includes actuators of a suspension adjustment unit, a handlebar adjustment unit, and a seat height adjustment unit of the motorcycle in the control target 252, and can adjust the hardness of the suspension, the height of the handlebar, and the seat height of the motorcycle.

The robot 100 executes processing of controlling the hardness of the suspension, the height of the handlebar, and the seat height of the motorcycle according to the preference of the user, the situation of the user, and the reaction of the user by Steps 1 to 5-2 below.

(Step 1) The robot 100 acquires the state of the user 10, the emotion value of the user 10, the emotion value of the robot 100, and the history data 222. Specifically, processing similar to above steps S100 to S103 is performed to acquire the state of the user 10, the emotion value of the user 10, the emotion value of the robot 100, and the history data 222. The state of the user 10 also includes the physique of the user 10 and the presence or absence of tandem rider.

(Step 2) The robot 100 acquires preference of the user 10 regarding control of the motorcycle.

Specifically, the behavior determination unit 236 determines, as a behavior of the robot 100, an utterance to the user 10 to ask a question about a preference regarding the control of the motorcycle, and the behavior control unit 250 controls a speaker, which is one of the control targets 252 and makes an utterance to the user 10 to ask the question about the preference regarding the control of the motorcycle. The user state recognition unit 230 recognizes the preference of the user 10 regarding the control of the motorcycle based on the information (for example, the user's answer) analyzed by the sensor module unit 210. Note that an example of the preference of the user regarding the control of the hardness of the suspension, the height of the handlebar, and the seat height of the motorcycle is such as “I WANT TO IMPROVE THE FOOT REACHABILITY WHEN THE MOTORCYCLE IS STOPPED”.

(Step 3) The robot 100 determines specific control regarding the hardness of the suspension, the height of the handlebar, and the seat height of the motorcycle proposed to the user 10.

Specifically, the behavior determination unit 236 adds a fixed sentence such as “AT THIS TIME, WHAT IS THE CONTROL OF THE MOTORCYCLE RECOMMENDED TO THE USER?” to the text representing the preference of the user 10 regarding the control of the motorcycle, the emotion of the user 10, the emotion of the robot 100, and the content stored in the history data 222, inputs the text into the sentence generation model, and acquires the recommended content regarding the control of the motorcycle. At this time, it is possible to propose a specific control on the motorcycle suitable for the user 10 by taking into consideration not only the preference of the user 10 regarding the control of the motorcycle but also the emotion of the user 10 and the history data 222. Furthermore, by taking into consideration the emotion of the robot 100, it is possible to make the user 10 feel that the robot 100 has an emotion.

An example of the specific control on the motorcycle is “RELATIVELY SOFTENING THE SUSPENSION AND RELATIVELY LOWERING THE SEAT HEIGHT WHEN THE MOTORCYCLE IS STOPPED”.

(Step 4) The robot 100 proposes the specific control on the motorcycle determined in Step 3 to the user 10, and acquires the reaction of the user 10.

Specifically, the behavior determination unit 236 determines, as a behavior of the robot 100, an utterance to the user 10 for proposing the specific control on the motorcycle, and the behavior control unit 250 controls a speaker, which is one of the control targets 252 and makes an utterance to the user 10 for proposing the specific control on the motorcycle. Note that, in Step 4, in addition to the utterance, specific control on the motorcycle, for example, “RELATIVELY SOFTENING THE SUSPENSION AND RELATIVELY LOWERING THE SEAT HEIGHT WHEN THE MOTORCYCLE IS STOPPED” or the like may be executed.

The user state recognition unit 230 recognizes the state of the user 10 based on the information analyzed by the sensor module unit 210, and the emotion determination unit 232 determines an emotion value indicating the emotion of the user 10 based on the information analyzed by the sensor module unit 210 and the state of the user 10 recognized by the user state recognition unit 230.

The behavior determination unit 236 determines whether the reaction of the user 10 is positive based on the state of the user 10 recognized by the user state recognition unit 230 and the emotion value indicating the emotion of the user 10, and determines whether to execute the specific control on the motorcycle proposed to the user 10 or to propose another control on the motorcycle to the user 10 as the behavior of the robot 100.

(Step 5-1) In a case where the reaction of the user 10 is positive, the robot 100 executes the specific proposed control related to the motorcycle.

Specifically, in a case where it is determined to execute the specific control on the motorcycle proposed to the user 10 as the behavior of the robot 100, the behavior control unit 250 controls the actuators of the suspension adjustment unit, the handlebar adjustment unit, and
the seat height adjustment unit of the motorcycle, which are one of the control targets 252, to execute the specific control on the motorcycle proposed to the user 10. In a case where the specific control on the motorcycle is executed in Step 4 described above, Step 5-1 may be skipped.

(Step 5-2) In a case where the reaction of the user 10 is not positive, the robot 100 determines another control on the motorcycle to be proposed to the user 10.

Specifically, in a case where it is determined to propose another control on the motorcycle to the user 10 as the behavior of the robot 100, the behavior determination unit 236 adds a fixed sentence such as “ARE THERE ANY OTHER CONTROLS ON THE MOTORCYCLE RECOMMENDED TO THE USER?” to the text representing the preference of the user 10 regarding the control on the motorcycle, the emotion of the user 10, the emotion of the robot 100, and the content stored in the history data 222, inputs the text to a sentence generation model, and acquires the recommended content regarding another control on the motorcycle. An example of another control on the motorcycle is, for example, “RAISING THE HEIGHT OF THE HANDLEBAR OF THE MOTORCYCLE”. Then, the processing returns to Step 4 described above, and the processing of Steps 4 to 5-2 described above is repeated until it is determined to execute the specific control on the motorcycle proposed to the user 10.

In this regard, the robot 100 can execute control on the motorcycle according to the preference of the user 10, the situation of the user 10, and the reaction of the user 10.

Note that, in the other embodiments described above, the aspect has been described in which the robot 100 has a conversation with the user 10, acquires the preference of the user 10 regarding the control on the motorcycle, and the like, and controls the motorcycle according to the preference of the user 10. However, after the preference of the user 10 regarding the control on the motorcycle is acquired once, the control on the motorcycle may be executed according to the preference of the user 10, the situation of the user 10, and the reaction of the user 10 without having to have a conversation with the user.

In the other embodiments described above, the aspect in which the suspension adjustment unit, the handlebar adjustment unit, and the seat height adjustment unit are provided in the motorcycle has been described. However, the disclosure is not limited to this, and the disclosure is also applicable to a motorcycle provided with at least one of the suspension adjustment unit, the handlebar adjustment unit, or the seat height adjustment unit.

With regard to the above embodiments, the following supplementary notes are further disclosed.

### Supplementary Note 1

A behavior control system including
a user state recognition unit that recognizes a user state including a behavior of a user,
an emotion determination unit that determines an emotion of the user or an emotion of a robot,
and a behavior determination unit that determines a behavior of the robot corresponding to the user state and the emotion of the user or the emotion of the robot based on a sentence generation model having an interaction function of causing the user to interact with the robot, in which
the behavior determination unit determines control on an actuator provided on a motorcycle as the behavior of the robot.

### Supplementary Note 2

The behavior control system according to Supplementary Note 1, in which the actuator is any of a first actuator capable of adjusting hardness of a suspension of the motorcycle, a second actuator capable of adjusting a height of a handlebar of the motorcycle, and a third actuator capable of adjusting a height of a seat of the motorcycle.

### Other Embodiment 12

As another embodiment, the robot 100 may be applied to a robot mounted on a device such as an electrical appliance, a computer, an automobile, a motorcycle, or a toy, or the robot 100 may control a device such as an electrical appliance, a computer, an automobile, a motorcycle, or a toy. Furthermore, the robot 100 may be mounted on a stuffed toy, or may be applied to a control device connected to a control target device (speaker or camera) mounted on the stuffed toy in a wireless or wired manner. Other embodiments are specifically configured as follows. For example, the robot 100 may be applied to control of fittings (AI fitting, interactive fitting) such as a door or a sash.

In one example, the fitting controlled by the robot 100 includes an opening-degree adjustment mechanism that adjusts the opening degree of the fitting, and an ultrasonic generator that generates insect repelling ultrasonic waves around the fitting.

In addition, in another example, the fitting controlled by the robot 100 includes an intercom capable of interacting with a visitor.

In one example, the robot 100 executes processing of adjusting the opening degree of a fitting and generating insect repelling ultrasonic waves around the fitting according to the preference of the user, the situation of the user, and the reaction of the user by Steps 1 to 5-2 below.

In addition, in another example, the robot 100 executes processing of responding to a visitor such as a door-to-door salesman according to preference of the user, the situation of the user, and the reaction of the user by Steps 1 to 5-2 below.

(Step 1) In one example and another example, the robot 100 acquires the state of the user 10, the emotion value of the user 10, the emotion value of the robot 100, and the history data 222. Specifically, processing similar to above steps S100 to S103 is performed to acquire the state of the user 10, the emotion value of the user 10, the emotion value of the robot 100, and the history data 222.

(Step 2) In one example, the robot 100 acquires the preference of the user 10 regarding the opening degree of the fitting and the insect repellent.

Specifically, the behavior determination unit 236 determines, as a behavior of the robot 100, an utterance to the user 10 to ask a question about a preference regarding the opening degree of the fitting and the insect repellent, and the behavior control unit 250 controls the control target 252 and makes an utterance to the user 10 to ask the question about the preference regarding the opening degree of the fitting and the insect repellent. The user state recognition unit 230 recognizes the preference of the user 10 regarding the opening degree of the fitting and the insect repellent based on the information (for example, the user's answer) analyzed by the sensor module unit 210.

In addition, in other examples, the robot 100 acquires preferences of the user 10 regarding visitors.

Specifically, the behavior determination unit 236 determines, as a behavior of the robot 100, an utterance to the user 10 to ask a question about a preference regarding the visitor, and the behavior control unit 250 controls the control target 252 and makes an utterance to the user 10 to ask the question about the preference regarding the visitor. The user state recognition unit 230 recognizes the preference of the user 10 regarding the visitor based on the information (for example, the user's answer) analyzed by the sensor module unit 210.

(Step 3) In one example, the robot 100 determines the opening degree of the fitting proposed to the user 10 and the necessity of insect repellent.

Specifically, the behavior determination unit 236 adds fixed sentences such as “AT THIS TIME, WHAT IS THE OPENING DEGREE OF THE FITTING RECOMMENDED TO THE USER?” and “AT THIS TIME, IS INSECT REPELLENT NECESSARY?” to the text representing the preference of the user 10 regarding the opening degree of the fitting and insect repellent, the emotion of the user 10, the emotion of the robot 100, and the content stored in the history data 222, inputs the text into a sentence generation model, and acquires the recommended content regarding the opening degree of the fitting and the content regarding the necessity of insect repellent. At this time, it is possible to propose an opening degree of the fitting and a necessity of insect repellent suitable for the user 10 by taking into consideration not only the preference of the user 10 regarding the opening degree of the fitting and insect repellent but also the emotion of the user 10 and the history data 222. Furthermore, by taking into consideration the emotion of the robot 100, it is possible to make the user 10 feel that the robot 100 has an emotion.

Furthermore, in another example, the robot 100 determines response content to a visitor to be proposed to the user 10.

Specifically, the behavior determination unit 236 adds a fixed sentence such as “AT THIS TIME, WHAT IS THE RESPONSE CONTENT TO A VISITOR RECOMMENDED TO THE USER?” to the text representing the preference of the user 10 regarding the visitor, the emotion of the user 10, the emotion of the robot 100, and the content stored in the history data 222, inputs the text into the sentence generation model, and acquires the recommended content regarding the response content to the visitor. At this time, it is possible to propose the response content to the visitor suitable for the user 10 by taking into consideration not only the preference of the user 10 regarding the visitor but also the emotion of the user 10 and the history data 222.

Furthermore, by taking into consideration the emotion of the robot 100, it is possible to make the user 10 feel that the robot 100 has an emotion.

(Step 4) In one example, the robot 100 proposes the opening degree of the fitting and the necessity of insect repellent determined in Step 3 to the user 10, and acquires the reaction of the user 10.

Specifically, the behavior determination unit 236 determines, as a behavior of the robot 100, an utterance to the user 10 for proposing the opening degree of the fitting and the necessity of insect repellent, and the behavior control unit 250 controls the control target 252 and makes an utterance to the user 10 for proposing the opening degree of the fitting and the necessity of insect repellent. The user state recognition unit 230 recognizes the state of the user 10 based on the information analyzed by the sensor module unit 210, and the emotion determination unit 232 determines an emotion value indicating the emotion of the user 10 based on the information analyzed by the sensor module unit 210 and the state of the user 10 recognized by the user state recognition unit 230.

The behavior determination unit 236 determines whether the reaction of the user 10 is positive based on the state of the user 10 recognized by the user state recognition unit 230 and the emotion value indicating the emotion of the user 10, adjusts the opening degree of the fitting proposed to the user 10 as the behavior of the robot 100, and determines whether to execute, if necessary, processing of generating an insect repelling ultrasonic waves around the fitting or to propose another opening degree of the fitting or the necessity of insect repellent to the user 10.

Furthermore, in another example, the robot 100 proposes the response content to the visitor determined in Step 3 to the user 10, and acquires the reaction of the user 10.

Specifically, the behavior determination unit 236 determines, as a behavior of the robot 100, an utterance to the user 10 for proposing the response content to the visitor, and the behavior control unit 250 controls the control target 252 and makes an utterance to the user 10 for proposing the response content to the visitor. The user state recognition unit 230 recognizes the state of the user 10 based on the information analyzed by the sensor module unit 210, and the emotion determination unit 232 determines an emotion value indicating the emotion of the user 10 based on the information analyzed by the sensor module unit 210 and the state of the user 10 recognized by the user state recognition unit 230.

The behavior determination unit 236 determines whether the reaction of the user 10 is positive based on the state of the user 10 recognized by the user state recognition unit 230 and the emotion value indicating the emotion of the user 10, and determines, as the behavior of the robot 100, whether to execute processing of responding to a visitor with a response content proposed to the user 10 or to propose a response content to another visitor to the user 10.

(Step 5-1) In one example, in a case where the reaction of the user 10 is positive, the robot 100 adjusts the proposed opening degree of the fitting, and executes processing of generating insect repelling ultrasonic waves around the fitting according to necessity.

Specifically, in a case where it is determined that the processing of adjusting the opening degree of the fitting proposed to the user 10 is executed as the behavior of the robot 100, the behavior control unit 250 controls the opening-degree adjustment mechanism as the control target 252 such that the opening degree of the fitting becomes the proposed opening degree for the user 10. In addition, in a case where it is determined that the processing of generating the insect repelling ultrasonic waves around the fitting proposed to the user 10 is executed as the behavior of the robot 100, the behavior control unit 250 controls the ultrasonic generator as the control target 252 such that the insect repelling ultrasonic waves are generated around the fitting proposed to the user 10.

Furthermore, in another example, in a case where the reaction of the user 10 is positive, the robot 100 executes processing of responding to the visitor with the proposed response content.

Specifically, in a case where it is determined to execute processing of responding to the visitor with the response content proposed to the user 10 as the behavior of the robot 100, the behavior control unit 250 controls the speaker of the intercom as the control target 252 to respond to the visitor with the response content proposed to the user 10.

(Step 5-2) In one example, in a case where the reaction of the user 10 is not positive, the robot 100 determines another opening degree of the fitting and necessity of insect repellent to be proposed to the user 10.

Specifically, in a case where it is determined, as a behavior of the robot 100, to propose to the user 10 another opening degree of the fitting and necessity of insect repellent, the behavior determination unit 236 adds fixed sentences such as “IS THERE ANOTHER OPENING DEGREE OF THE FITTING RECOMMENDED TO THE USER?” and “AT THIS TIME, IS INSECT REPELLENT NECESSARY?” to the text representing the preference of the user 10 regarding the opening degree of the fitting and insect repellent, the emotion of the user 10, the emotion of the robot 100, and the content stored in the history data 222, inputs the text into a sentence generation model, and acquires the recommended content regarding the opening degree of the fitting and the content regarding the necessity of insect repellent. Then, the processing returns to Step 4, and the processing of Steps 4 to 5-2 is repeated until it is determined that the opening degree of the fitting proposed to the user 10 is adjusted and the processing of generating the insect repelling ultrasonic waves around the fitting is executed according to necessity.

Furthermore, in another example, in a case where the reaction of the user 10 is not positive, the robot 100 determines the response content to another visitor to be proposed to the user 10.

Specifically, in a case where it is determined to propose another response content to another visitor to the user 10 as the behavior of the robot 100, the behavior determination unit 236 adds a fixed sentence such as “ARE THERE ANY OTHER RESPONSE CONTENT TO THE VISITOR RECOMMENDED TO THE USER?” to the text representing the preference of the user 10 regarding the visitor, the emotion of the user 10, the emotion of the robot 100, and the content stored in the history data 222, inputs the text to a sentence generation model, and acquires the recommended content regarding the response content to the visitor. Then, the processing returns to Step 4 described above, and the processing of Steps 4 to 5-2 described above is repeated until it is determined to execute the processing of responding to the visitor with the response content proposed to the user 10.

In this regard, in one example, the robot 100 can execute processing of adjusting the opening degree of a fitting and generating insect repelling ultrasonic waves around the fitting according to the preference of the user, the situation of the user, and the reaction of the user.

In addition, in another example, the robot 100 can execute processing of responding to a visitor such as a door-to-door salesman according to the preference of the user, the situation of the user, and the reaction of the user.

With regard to the above embodiments, the following supplementary notes are further disclosed.

### Supplementary Note 1

A behavior control system including
a user state recognition unit that recognizes a user state including a behavior of a user, an emotion determination unit that determines an emotion of the user or an emotion of a robot,
and a behavior determination unit that determines a behavior of the robot corresponding to the user state and the emotion of the user or the emotion of the robot based on a sentence generation model having an interaction function of causing the user to interact with the robot, in which
the behavior determination unit determines whether to generate an insect repelling ultrasonic wave around a fitting based on an interaction result with the user by the sentence generation model, or determines a response content to a visitor based on an interaction result with the user by the sentence generation model.

### Supplementary Note 2

The behavior control system according to Supplementary Note 1, in which the behavior determination unit determines an opening degree of the fitting and determines whether to generate an insect repelling ultrasonic wave around the fitting based on the interaction result with the user by the sentence generation model.

### Supplementary Note 3

The behavior control system according to Supplementary Note 1 or 2, in which the robot is mounted on a stuffed toy or is connected to a control target device mounted on the stuffed toy in a wireless or wired manner.

### Other Embodiment 13

As another embodiment, the robot 100 may be applied to a robot mounted on a device such as an electrical appliance, a computer, an automobile, a motorcycle, or a toy, or the robot 100 may control a device such as an electrical appliance, a computer, an automobile, a motorcycle, or a toy. Furthermore, the robot 100 may be mounted on a stuffed toy, or may be applied to a control device connected to a control target device (speaker or camera) mounted on the stuffed toy in a wireless or wired manner. Other embodiments are specifically configured as follows. For example, the robot 100 may be applied to control of a menu creation system that proposes content of a menu of a dish.

The menu creation system controlled by the robot 100 includes a storage unit that stores menu history data, an acquisition unit that acquires information regarding a temperature, humidity, or a season, information regarding ingredients, information regarding an inventory of a refrigerator, information regarding a physical condition or illness of a user, or the like, and a speaker that outputs a voice. As the acquisition unit, the communication processing unit 280 connected to an external device in a wired or wireless manner can be used. Furthermore, the robot 100 may include a sensor that detects temperature and humidity. In this case, a sensor can be used as the acquisition unit. Furthermore, the menu creation system may include a display unit that displays content of menus.

The robot 100 executes processing of creating a menu and outputting the content of the menu according to the preference of the user, the situation of the user, and the reaction of the user by Steps 1 to 5-2 below.

(Step 1) The robot 100 acquires the state of the user 10, the emotion value of the user 10, the emotion value of the robot 100, and the history data 222. Specifically, processing similar to above steps S100 to S103 is performed to acquire the state of the user 10, the emotion value of the user 10, the emotion value of the robot 100, and the history data 222.

(Step 2) The robot 100 acquires the preference of the user 10 regarding favorite ingredients, tastes, genres of cuisine, and the like.

Specifically, the behavior determination unit 236 determines, as a behavior of the robot 100, an utterance to the user 10 to ask a question about a preference regarding a favorite ingredient, a taste, a genre of cuisine, or the like, and the behavior control unit 250 controls the control target 252 and makes an utterance of asking the user 10 the question about the preference regarding a favorite ingredient, a taste, a genre of cuisine, and the like. The user state recognition unit 230 recognizes the preference of the user 10 regarding the favorite ingredients, tastes, genres of cuisine, and the like based on the information (for example, the user's answer) analyzed by the sensor module unit 210. For example, the robot 100 utters to the user 10 questions such as “WHAT IS YOUR FAVORITE INGREDIENT?”, “WHAT IS YOUR FAVORITE TASTE?”, and “WHAT IS YOUR FAVORITE GENRE OF CUISINE?”. Then, the robot 100 recognizes the preference of the user 10 regarding favorite ingredients, tastes, genres of cuisine, or the like based on answers from the user 10 such as “I LIKE TOMATOES”, “I LIKE SPICY FOOD”, or “I LIKE JAPANESE CUISINE”.

(Step 3) The robot 100 determines a content of a menu to be proposed to the user 10.

Specifically, the behavior determination unit 236 adds a fixed sentence such as “AT THIS TIME, WHAT IS THE MENU RECOMMENDED TO THE USER?” to the text representing the preference of the user 10 regarding the favorite ingredients, tastes, genres of cuisine, or the like, the emotion of the user 10, the emotion of the robot 100, and the content stored in the history data 222, inputs the text into the sentence generation model, and acquires the recommended content regarding the favorite ingredients, tastes, genres of cuisine, or the like. At this time, it is possible to propose the menu suitable for the emotion of the user 10 at that time by taking into consideration not only the preference of the user 10 regarding the favorite ingredients, tastes, genres of cuisine, or the like but also the emotion of the user 10 and the history data 222. Furthermore, by taking into consideration the emotion of the robot 100, it is possible to make the user 10 feel that the robot 100 has an emotion. For example, in a case where it is determined that the favorite ingredient of the user 10 is tomato and the user 10 is tired, the robot 100 performs control to create a menu having a fatigue recovery effect using tomato and output the content of the menu.

(Step 4) The robot 100 proposes the content of the menu determined in Step 3 to the user 10 and acquires the reaction of the user 10.

Specifically, the behavior determination unit 236 determines, as a behavior of the robot 100, an utterance to the user 10 for proposing the determined content of the menu, and the behavior control unit 250 controls the control target 252 and makes an utterance to the user 10 for proposing the determined content of the menu. The user state recognition unit 230 recognizes the state of the user 10 based on the information analyzed by the sensor module unit 210, and the emotion determination unit 232 determines an emotion value indicating the emotion of the user 10 based on the information analyzed by the sensor module unit 210 and the state of the user 10 recognized by the user state recognition unit 230.

The behavior determination unit 236 determines whether the reaction of the user 10 is positive based on the state of the user 10 recognized by the user state recognition unit 230 and the emotion value indicating the emotion of the user 10, and determines whether to execute processing of outputting the content of the menu proposed to the user 10 or to propose content of another menu to the user 10 as the behavior of the robot 100.

(Step 5-1) In a case where the reaction of the user 10 is positive, the robot 100 executes processing of outputting the proposed content of the menu.

Specifically, in a case where it is determined to execute processing of outputting the proposed content of the menu for the user 10 as the behavior of the robot 100, the behavior control unit 250 controls the menu creation system as the control target 252 to execute output of the content of the menu proposed to the user 10.

(Step 5-2) In a case where the reaction of the user 10 is not positive, the robot 100 determines output of the content of another menu to be proposed to the user 10. For example, in a case where it is determined that the tension of the emotion of the user 10 is not too high, the behavior determination unit 236 performs control to output the content of another menu.

Specifically, for example, the robot 100 utters a question such as “HOW ABOUT A HOT POT DISH?”. Then, the robot 100 performs control to output the content of another menu in a case where it is determined that a deliberate reaction such as “HMM.” from the user 10 or a reaction of the user 10 to an utterance of the robot 100 is slow.

In a case where it is determined to propose another content of the menu to the user 10 as the behavior of the robot 100, the behavior determination unit 236 adds a fixed sentence such as “ARE THERE ANY OTHER MENU RECOMMENDED TO THE USER?” to the text representing the preference of the user 10 regarding the favorite ingredients, tastes, genres of cuisine, or the like, the emotion of the user 10, the emotion of the robot 100, and the content stored in the history data 222, inputs the text to a sentence generation model, and acquires the recommended content regarding the favorite ingredients, tastes, genres of cuisine, or the like. Then, the processing returns to Step 4 described above, and the processing of Steps 4 to 5-2 described above is repeated until it is determined to execute the processing of outputting the content of the menu proposed to the user 10.

In this regard, the robot 100 can execute processing of creating a menu and outputting the content of the menu according to the preference of the user, the situation of the user, and the reaction of the user.

The behavior determination unit 236 may perform control such that the content of the menu to be output by the robot 100 is changed using the information regarding the temperature, the humidity, or the season. The information regarding the temperature, the humidity, or the season may be acquired via the communication processing unit 280 connected to an external device, or may be detected and acquired by a sensor provided in the robot 100.

Specifically, for example, in a case where it is determined that the season is winter and the temperature on that day is a cold day lower than a predetermined temperature, the behavior determination unit 236 performs control to propose the content of the menu of a dish that warms the body, such as a dish for thin sliced beef or a pot dish, according to the preference of the user, the situation of the user, and the reaction of the user. Furthermore, in a case where it is determined that the season is summer and the temperature on that day is a hot day higher than the predetermined temperature, the behavior determination unit 236 performs control to propose the content of a simple dish menu such as Somen according to the preference of the user, the situation of the user, and the reaction of the user. Furthermore, in a case where it is determined that the day on which the menu is output is Christmas, the behavior determination unit 236 performs control to propose a menu of a Christmas dish according to the preference of the user, the situation of the user, and the reaction of the user.

Furthermore, the behavior determination unit 236 may perform control such that the content of the menu to be output by the robot 100 is changed according to the preference of the user, the situation of the user, and the reaction of the user by using the information regarding the ingredient to be used. As the information regarding the ingredient to be used, information acquired by the robot 100 from the interaction with the user 10 is used.

Specifically, for example, in a case where it is determined that the ingredient to be used is meat, the behavior determination unit 236 performs control to change the menu to a menu using meat according to the preference of the user, the situation of the user, and the reaction of the user.

Furthermore, the behavior determination unit 236 may perform control such that the content of the menu to be output by the robot 100 is changed according to the preference of the user, the situation of the user, and the reaction of the user by using the information regarding the inventory of the refrigerator. The information regarding the inventory of the refrigerator may be acquired by the robot 100 from the interaction with the user 10 or may be acquired via the communication processing unit 280 connected to a camera or the like mounted on the refrigerator.

Specifically, for example, in a case where it is determined that there are yellowtail, Japanese radish, and carrot in the refrigerator, the behavior determination unit 236 performs control to change the menu to a menu using yellowtail, Japanese radish, and carrot according to the preference of the user, the situation of the user, and the reaction of the user.

Furthermore, the behavior determination unit 236 may perform control such that the content of the menu to be output by the robot 100 is changed according to the preference of the user, the situation of the user, and the reaction of the user by using the information regarding the physical condition and illness of the user. The information regarding the physical condition and the illness of the user may be acquired by the robot 100 from the interaction with the user 10 or may be acquired via the communication processing unit 280 connected to the wearable terminal worn by the user 10. Furthermore, the information regarding the illness of the user may be acquired using the information regarding the medicine that the user 10 is taking.

Specifically, for example, in a case where it is determined that the user is tired, the behavior determination unit 236 performs control to change the content to a content of a menu having a fatigue recovery effect according to the preference of the user, the situation of the user, and the reaction of the user. Furthermore, in a case where it is determined that the blood pressure is high, the behavior determination unit 236 performs control such that the menu is changed to a menu for lowering the blood pressure according to the preference of the user, the situation of the user, and the reaction of the user. Furthermore, in a case where it is determined that the user 10 is taking a medicine for diabetes, the behavior determination unit 236 performs control such that the menu is changed to a menu for lowering the blood glucose level according to the preference of the user, the situation of the user, and the reaction of the user.

Furthermore, the behavior determination unit 236 performs control such that the content of the menu to be output by the robot 100 is changed using the menu history data of the user. Specifically, the robot 100 stores the content of the menu output to the user in the storage unit as menu history data. Then, the behavior determination unit 236 performs control to change the content of the menu to exclude a menu for which a predetermined period has not elapsed since the output.

The behavior determination unit 236 selects a genre of cuisine to be output based on the emotion of the user 10 or the emotion of the robot 100, and controls the robot 100 to output the menu included in the selected genre. For example, the cuisines to be output may be classified into genres such as Japanese, Western, Chinese, Asian, and ethnic, and a menu included in the genre of cuisine to be output may be output.

With regard to the above embodiments, the following supplementary notes are further disclosed.

### Supplementary Note 1

A behavior control system including
a user state recognition unit that recognizes a user state including a behavior of a user,
an emotion determination unit that determines an emotion of the user or an emotion of a robot,
and a behavior determination unit that determines a behavior of the robot corresponding to the user state and the emotion of the user or the emotion of the robot based on a sentence generation model having an interaction function of causing the user to interact with the robot, in which
the behavior determination unit causes the robot to output a menu content based on the emotion of the user or the emotion of the robot.

### Supplementary Note 2

The behavior control system according to Supplementary Note 1, further including
an acquisition unit that acquires information regarding a temperature, humidity, or a season, and
the behavior determination unit performs control to change content of a menu to be output by the robot, using the information regarding the temperature, the humidity, or the season.

### Supplementary Note 3

The behavior control system according to Supplementary Note 1, further including an acquisition unit that acquires information regarding an ingredient, and
the behavior determination unit performs control to change content of a menu to be output by the robot, using the information regarding the ingredient to be used.

### Supplementary Note 4

The behavior control system according to Supplementary Note 1, further including
an acquisition unit that acquires information regarding an inventory of a refrigerator, in which
the behavior determination unit performs control to change content of a menu to be output by the robot, using the information regarding the inventory of the refrigerator.

### Supplementary Note 5

The behavior control system according to Supplementary Note 1, further including
an acquisition unit that acquires information regarding a physical condition of a user, and
the behavior determination unit performs control to change content of a menu to be output by the robot, using the information regarding the physical condition of the user.

### Supplementary Note 6

The behavior control system according to Supplementary Note 1, in which
the behavior determination unit performs control to change content of a menu to be output by the robot, using menu history data of a user.

### Supplementary Note 7

The behavior control system according to Supplementary Note 1, in which the robot is mounted on a stuffed toy or is connected to a control target device mounted on the stuffed toy in a wireless or wired manner.

### Other Embodiment 14

As another embodiment, the robot 100 may be applied to a robot mounted on a device such as an electrical appliance, a computer, an automobile, a motorcycle, or a toy, or the robot 100 may control a device such as an electrical appliance, a computer, an automobile, a motorcycle, or a toy. Furthermore, the robot 100 may be mounted on a stuffed toy, or the robot 100 may control a control target device (for example, a speaker or a camera) mounted on the stuffed toy. In a case where the robot 100 controls the control target device, for example, the robot 100 is connected to the control target device in a wireless or wired manner.

Furthermore, as another embodiment, the robot 100 described above may be mounted on a measurement device that measures a numerical value of body composition, or the robot 100 may control a control target device (for example, a speaker or a camera) mounted on the measurement device. In a case where the robot 100 controls the control target device, for example, the robot 100 is connected to the control target device in a wireless or wired manner. The numerical values of the body composition are numerical values relating to the composition of the body, such as weight, height, body fat percentage, muscle mass, skeletal muscle percentage, bone mass, basal metabolism, visceral fat level, and BMI. Examples of the measurement device include a scale, a height meter, and a body composition meter. Note that, in a case where the robot 100 is applied to a measurement device, for example, it is assumed that there are a plurality of users including family members, friends, and the like.

The measurement device includes a measuring unit that measures a numerical value of the body composition. The measuring unit includes various sensors, and measures a numerical value of a body composition by measuring a weight, a dimension, an electric resistance value, and the like of the user 10 using the sensors. In addition, the measuring unit is controlled by the robot 100, and the robot 100 acquires a measurement result (that is, numerical data) measured by the measurement unit. Note that the measuring unit may be controlled by a control unit different from the robot 100.

The robot 100 executes processing of presenting a response operation to the user whose numerical value of the body composition has been measured according to the preference of the user, the situation of the user, and the reaction of the user by Steps 1 to 5 below. Specifically, according to the preference of the user, the situation of the user, and the reaction of the user, the robot 100 executes, for example, processing of presenting a response operation of praising, encouraging, or reproving the user who has measured the numerical value of the body composition to the user 10 by Steps 1 to 5 below. As the response operations, praising, encouraging, and reproving are representative operations; however, it is not limited to these, and may include various response operations (for example, admonishing, leaning without speaking, and the like).

(Step 1) The robot 100 acquires the state of the user 10, the emotion value of the user 10, the emotion value of the robot 100, and the history data 222. Specifically, processing similar to above steps S100 to S103 is performed to acquire the state of the user 10, the emotion value of the user 10, the emotion value of the robot 100, and the history data 222. Further, the robot 100 acquires a measurement result by the measuring unit.

(Step 2) The robot 100 acquires, for example, the preference of the user 10 regarding responses from others and regarding the body.

Specifically, the behavior determination unit 236 determines, as a behavior of the robot 100, an utterance to the user 10 to ask about the preference of the user 10 regarding responses from others and regarding the body, and the behavior control unit 250 controls the control target 252 and makes an utterance to the user 10 to ask about the preference of the user 10 regarding responses from others and regarding the body. The user state recognition unit 230 recognizes the preference of the user 10 regarding responses from others and regarding the body based on the information (for example, the user's answer) analyzed by the sensor module unit 210.

Examples of the preference regarding the body include preference regarding body shape (for example, a thin type, an obese type, or the like), physique (for example, muscular, or the like), and exercise involving movement of the body. Examples of the preference regarding the response from another person include whether the user prefers to be complimented by another person, whether the user prefers to be encouraged by another person, and whether the user prefers to be reprimanded by another person.

(Step 3) The robot 100 determines a response operation to be presented to the user 10.

Specifically, the behavior determination unit 236 adds a fixed sentence such as “AT THIS TIME, WHAT IS THE RESPONSE OPERATION RECOMMENDED TO THE USER?” to text representing a measurement result by the measuring unit, a response from another person and a preference regarding the body of the user 10, an emotion of the user 10, an emotion of the robot 100, and content stored in the history data 222, inputs the text to a sentence generation model, and acquires recommended operation content. At this time, it is possible to provide the response operation suitable for the user 10 by taking into consideration not only the response from others and the preference of the user 10 regarding the body but also the emotion of the user 10 and the history data 222. Furthermore, by taking into consideration the emotion of the robot 100, it is possible to make the user 10 feel that the robot 100 has an emotion.

(Step 4) The robot 100 presents the response operation determined in Step 3 to the user 10 and acquires the reaction of the user 10.

Specifically, the behavior determination unit 236 determines, as a behavior of the robot 100, an utterance to the user 10 for presenting a response operation, and the behavior control unit 250 controls the control target 252 and performs an utterance to the user 10 for presenting the response operation. The user state recognition unit 230 recognizes the state of the user 10 based on the information analyzed by the sensor module unit 210, and the emotion determination unit 232 determines an emotion value indicating the emotion of the user 10 based on the information analyzed by the sensor module unit 210 and the state of the user 10 recognized by the user state recognition unit 230.

The behavior determination unit 236 determines whether the reaction of the user 10 is positive based on the state of the user 10 recognized by the user state recognition unit 230 and the emotion value indicating the emotion of the user 10.

(Step 5) In a case where the reaction of the user 10 is not positive, the robot 100 determines another response operation presented to the user 10.

Specifically, in a case where it is determined to present another response operation to the user 10 as the behavior of the robot 100, the behavior determination unit 236 adds a fixed sentence such as “ARE THERE ANY OTHER RESPONSE OPERATIONS RECOMMENDED TO THE USER?” to the text representing the measurement result by the measuring unit, the response from other persons to the user 10, the preference regarding the body, the emotion of the user 10, the emotion of the robot 100, and the content stored in the history data 222, inputs the text into a sentence generation model, and acquires the content regarding the recommended response operation. Then, the processing returns to Step 4 above, the response operation is presented to the user 10, and the processing of Steps 4 to 5 is repeated until it is determined that the reaction of the user 10 is positive.

In this regard, the robot 100 can execute processing of presenting a response operation to the user, whose numerical value of the body composition has been measured, in accordance with the preference of the user, the situation of the user, and the reaction of the user.

With regard to the above embodiments, the following supplementary notes are further disclosed.

### Supplementary Note 1

A behavior control system including
a user state recognition unit that recognizes a user state including a behavior of a user,
an emotion determination unit that determines an emotion of the user or an emotion of a robot,
and a behavior determination unit that determines a behavior of the robot corresponding to the user state and the emotion of the user or the emotion of the robot based on a sentence generation model having an interaction function of causing the user to interact with the robot, in which
the behavior determination unit determines, as the behavior, a response operation of the robot for the user whose numerical value of a body composition has been measured based on the sentence generation model.

### Supplementary Note 2

The behavior control system according to Supplementary Note 1, in which
the behavior determination unit determines, as the response operation, an operation of praising, encouraging, or reproving the user.

### Supplementary Note 3

The behavior control system according to Supplementary Note 1, in which the robot is mounted on a measurement device that measures a numerical value of body composition or controls a control target device mounted on the measurement device.

### Supplementary Note 4

The behavior control system according to Supplementary Note 1, in which the robot is mounted on a stuffed toy or controls a control target device mounted on the stuffed toy.

### Other Embodiment 15

As another embodiment, the robot 100 may be applied to a robot mounted on a device such as an electrical appliance, a computer, an automobile, a motorcycle, or a toy, or the robot 100 may control a device such as an electrical appliance, a computer, an automobile, a motorcycle, or a toy. Furthermore, the robot 100 may be mounted on a stuffed toy, or may be applied to a control device connected to a control target device (speaker or camera) mounted on the stuffed toy in a wireless or wired manner. Other embodiments are specifically configured as follows. For example, the robot 100 may be applied to control of an AI small talk system.

The AI small talk system controlled by the robot 100 functionally includes a news distribution unit that acquires and distributes news searched by the Internet search engine, and a comment adding unit that adds (for example, upload to the cloud) a comment on the distributed news.

The robot 100 executes processing of distributing news according to preference of the user, the situation of the user, and the reaction of the user by Steps 1 to 5-2 below. Furthermore, the robot 100 executes processing of giving a comment on the news distributed while having a conversation with the robot, by Steps 11 to 15-2 below.

(Step 1) The robot 100 acquires the state of the user 10, the emotion value of the user 10, the emotion value of the robot 100, and the history data 222. Specifically, processing similar to above steps S100 to S103 is performed to acquire the state of the user 10, the emotion value of the user 10, the emotion value of the robot 100, and the history data 222.

(Step 2) The robot 100 acquires a preference of the user 10 regarding news.

Specifically, the behavior determination unit 236 determines, as a behavior of the robot 100, an utterance to the user 10 to ask a question about a preference regarding the news, and the behavior control unit 250 controls the control target 252 and makes an utterance to the user 10 to ask the question about the preference regarding the news. The user state recognition unit 230 recognizes the preference of the user 10 regarding the news based on the information (for example, the user's answer) analyzed by the sensor module unit 210.

(Step 3) The robot 100 determines news proposed to the user 10.

Specifically, the behavior determination unit 236 adds a fixed sentence such as “AT THIS TIME, WHAT IS THE NEWS RECOMMENDED TO THE USER?” to the text representing the preference of the user 10 regarding the news, the emotion of the user 10, the emotion of the robot 100, and the content stored in the history data 222, inputs the text into the sentence generation model, and acquires the recommended content regarding the news. At this time, it is possible to propose the news suitable for the user 10 by taking into consideration not only the preference of the user 10 regarding the news but also the emotion of the user 10 and the history data 222. Furthermore, by taking into consideration the emotion of the robot 100, it is possible to make the user 10 feel that the robot 100 has an emotion.

(Step 4) The robot 100 proposes the news determined in Step 3 to the user 10 and acquires the reaction of the user 10.

Specifically, the behavior determination unit 236 determines, as a behavior of the robot 100, an utterance to the user 10 for proposing news, and the behavior control unit 250 controls the control target 252 and makes an utterance to the user 10 for proposing the news. The user state recognition unit 230 recognizes the state of the user 10 based on the information analyzed by the sensor module unit 210, and the emotion determination unit 232 determines an emotion value indicating the emotion of the user 10 based on the information analyzed by the sensor module unit 210 and the state of the user 10 recognized by the user state recognition unit 230.

The behavior determination unit 236 determines whether the reaction of the user 10 is positive based on the state of the user 10 recognized by the user state recognition unit 230 and the emotion value indicating the emotion of the user 10, and determines whether to execute processing of distributing news proposed to the user 10 or to propose another news to the user 10 as the behavior of the robot 100.

(Step 5-1) In a case where the reaction of the user 10 is positive, the robot 100 executes processing of distributing the proposed news.

Specifically, in a case where it is determined that the processing of distributing the proposed news to the user 10 is executed as the behavior of the robot 100, the behavior control unit 250 controls at least one of the display apparatus or the speaker, which is the control target 252, to execute the processing of distributing the news proposed to the user 10.

(Step 5-2) In a case where the reaction of the user 10 is not positive, the robot 100 determines another news to be proposed to the user 10.

Specifically, in a case where it is determined to propose another news to the user 10 as the behavior of the robot 100, the behavior determination unit 236 adds a fixed sentence such as “ARE THERE ANY OTHER NEWS RECOMMENDED TO THE USER?” to the text representing the preference of the user 10 regarding the news, the emotion of the user 10, the emotion of the robot 100, and the content stored in the history data 222, inputs the text to a sentence generation model, and acquires the recommended content regarding the news. Then, the processing returns to Step 4 described above, and the processing of Steps 4 to 5-2 described above is repeated until it is determined to execute the processing of distributing the news proposed to the user 10.

In this regard, the robot 100 can execute processing of distributing the news according to the preference of the user, the situation of the user, and the reaction of the user.

(Step 11) The robot 100 acquires the state of the user 10, the emotion value of the user 10, the emotion value of the robot 100, and the history data 222. Specifically, processing similar to above steps S100 to S103 is performed to acquire the state of the user 10, the emotion value of the user 10, the emotion value of the robot 100, and the history data 222.

(Step 12) The robot 100 acquires preference of the user 10 regarding the comment on the distributed news.

Specifically, the behavior determination unit 236 determines, as a behavior of the robot 100, an utterance to the user 10 to ask a question about a preference regarding a comment on the distributed news, and the behavior control unit 250 controls the control target 252 and makes an utterance to the user 10 to ask the question about the preference regarding a comment on the distributed news. The user state recognition unit 230 recognizes the preference of the user 10 regarding the comment on the distributed news based on the information (for example, the user's answer) analyzed by the sensor module unit 210.

(Step 13) The robot 100 determines a comment on the distributed news to be proposed to the user 10.

Specifically, the behavior determination unit 236 adds a fixed sentence such as “WHAT IS A COMMENT RECOMMENDED TO THE USER REGARDING THE DISTRIBUTED NEWS?” to the text representing the preference of the user 10 regarding the comment on the distributed news, the emotion of the user 10, the emotion of the robot 100, and the content stored in the history data 222, inputs the text into the sentence generation model, and acquires the recommended content regarding the comment on the distributed news. At this time, it is possible to propose the comment on the distributed news suitable for the user 10 by taking into consideration not only the preference of the user 10 regarding the comment on the distributed news but also the emotion of the user 10 and the history data 222. Furthermore, by taking into consideration the emotion of the robot 100, it is possible to make the user 10 feel that the robot 100 has an emotion.

(Step 14) The robot 100 proposes the comment on the distributed news determined in Step 13 to the user 10, and acquires the reaction of the user 10.

Specifically, the behavior determination unit 236 determines, as a behavior of the robot 100, an utterance to the user 10 for proposing a comment on the distributed news, and the behavior control unit 250 controls the control target 252 and makes an utterance to the user 10 for proposing the comment on the distributed news. The user state recognition unit 230 recognizes the state of the user 10 based on the information analyzed by the sensor module unit 210, and the emotion determination unit 232 determines an emotion value indicating the emotion of the user 10 based on the information analyzed by the sensor module unit 210 and the state of the user 10 recognized by the user state recognition unit 230. The behavior determination unit 236 determines whether the reaction of the user 10 is positive based on the state of the user 10 recognized by the user state recognition unit 230 and the emotion value indicating the emotion of the user 10, and determines, as the behavior of the robot 100, whether to execute processing of giving a comment on the distributed news proposed to the user 10 or to propose another comment on the distributed news to the user 10.

(Step 15-1) In a case where the reaction of the user 10 is positive, the robot 100 executes processing of giving a proposed comment on the distributed news.

Specifically, in a case where it is determined that the processing of giving the comment on the distributed news proposed to the user 10 is executed as the behavior of the robot 100, the behavior control unit 250 controls at least one of the display apparatus or the speaker, which is the control target 252, to execute the processing of giving the comment on the distributed news proposed to the user 10.

(Step 15-2) In a case where the reaction of the user 10 is not positive, the robot 100 determines another comment for the distributed news to be proposed to the user 10.

Specifically, in a case where it is determined that another comment on the distributed news is to be proposed to the user 10 as the behavior of the robot 100, the behavior determination unit 236 adds a fixed sentence such as “ARE THERE ANY OTHER COMMENTS RECOMMENDED TO THE USER ON THE DISTRIBUTED NEWS?” to the text representing the preference regarding the comment on the distributed news to the user 10, the emotion of the user 10, the emotion of the robot 100, and the content stored in the history data 222, inputs the text to a sentence generation model, and acquires recommended content regarding the comment on the distributed news. Then, the processing returns to Step 14, and
the processing of Steps 14 to 15-2 is repeated until it is determined to execute the processing of giving another comment to the distributed news proposed to the user 10.

In this regard, the robot 100 can execute processing of giving a comment on the distributed news while having a conversation according to the preference of the user, the situation of the user, and the reaction of the user.

With regard to the above embodiments, the following supplementary notes are further disclosed.

### Supplementary Note 1

A behavior control system including
a user state recognition unit that recognizes a user state including a behavior of a user,
an emotion determination unit that determines an emotion of the user or an emotion of a robot,
and a behavior determination unit that determines a behavior of the robot corresponding to the user state and the emotion of the user or the emotion of the robot based on a sentence generation model having an interaction function of causing the user to interact with the robot, in which
the behavior determination unit determines, as a behavior of the robot, to identify preferred news of the user, to distribute the identified news, and to add a comment on the distributed news.

### Supplementary Note 2

The behavior control system according to Supplementary Note 1, in which the robot is mounted on a stuffed toy or is connected to a control target device mounted on the stuffed toy in a wireless or wired manner.

### Other Embodiment 16

As another embodiment, the robot 100 may be applied to a robot mounted on a device such as an electrical appliance, a computer, an automobile, a motorcycle, or a toy, or the robot 100 may control a device such as an electrical appliance, a computer, an automobile, a motorcycle, or a toy. Furthermore, the robot 100 may be mounted on a stuffed toy, or may be applied to a control device connected to a control target device (speaker or camera) mounted on the stuffed toy in a wireless or wired manner. Other embodiments are specifically configured as follows. For example, the robot 100 may be applied to control of a driving lesson device that gives advice on driving of vehicles such as automobiles and motorcycles.

In a case where the robot 100 is applied to the driving lesson device, for example, the storage unit 220 includes driving skill data regarding a driving skill in addition to the reaction rule 221 and the history data 222. The driving skill data includes data regarding an appropriate traveling position of the host vehicle with respect to the traveling lane, data regarding appropriate acceleration/deceleration, and the like. Furthermore, the driving skill data may include appropriate operation timings of the accelerator pedal and the brake pedal, appropriate steering timing of the steering, an appropriate steering angle, and the like. Furthermore, the driving skill data may include data regarding a skill for parking the vehicle in a predetermined area such as a parking lot.

Furthermore, the robot 100 is configured to be able to communicate with vehicles such as an automobile and a motorcycle driven by the user, and acquires data of the vehicle at the time of driving. The history data 222 stored in the storage unit 220 includes driving data during driving of the user 10 as a history of behavior of user 10. As the driving data, for example, position information of the vehicle, a vehicle speed, a depression amount of an accelerator pedal, a depression amount of a brake pedal, a steering angle of a steering, and the like are stored as a set. Furthermore, data such as the line-of-sight direction, the heart rate, and the wakefulness level of the user 10 during driving may be stored as the driving data. Note that the driving skill data and the driving data may be stored in the server 300 or the like different from the storage unit 220.

The robot 100 executes processing of giving advice for improving the driving skill according to the preference of the user, the situation of the user, and the reaction of the user by Steps 1 to 5-2 below.

(Step 1) The robot 100 acquires the state of the user 10, the emotion value of the user 10, the emotion value of the robot 100, and the history data 222. Specifically, processing similar to above steps S100 to S103 is performed to acquire the state of the user 10, the emotion value of the user 10, the emotion value of the robot 100, and the history data 222. At this time, the driving data of the user 10 is acquired as the history data 222, and the driving tendency of the user 10 is ascertained.

(Step 2) The robot 100 acquires a preference of the user 10 regarding driving of the vehicle.

Specifically, the behavior determination unit 236 determines, as a behavior of the robot 100, an utterance to the user 10 to ask a question about a preference regarding the driving of the vehicle, and the behavior control unit 250 controls the control target 252 and makes an utterance to the user 10 to ask the question about the preference regarding the driving of the vehicle. The user state recognition unit 230 recognizes the preference of the user 10 regarding the driving of the vehicle based on the information (for example, the user's answer) analyzed by the sensor module unit 210.

(Step 3) The robot 100 determines a content of a driving to be proposed to the user 10.

Specifically, the behavior determination unit 236 adds a fixed sentence such as “WHAT KIND OF DRIVING DOES THE USER LIKE?” to the text representing the preference of the user 10 regarding the driving of the vehicle, the emotion of the user 10, the emotion of the robot 100, and the content stored in the history data 222, inputs the text into a sentence generation model, and acquires the recommended content regarding the driving of the vehicle. At this time, it is possible to propose the driving suitable for the user 10 by taking into consideration not only the preference of the user 10 regarding the driving of the vehicle but also the emotion of the user 10 and the history data 222. For example, the behavior determination unit 236 may change the form of advice based on the emotion of the user. In a case where the emotion of the user 10 is an unpleasant emotion such as “ANGER”, “GRIEF”, “DISCOMFORT”, “ANXIETY”, “SADNESS”, “WORRY”, or “SENSE OF EMPTINESS,” the aspect of the proposal to the user 10 may be changed depending on whether such an unpleasant emotion is present. Furthermore, by taking into consideration the emotion of the robot 100, it is possible to make the user 10 feel that the robot 100 has an emotion.

(Step 4) The robot 100 proposes the content of the driving determined in Step 3 to the user 10 and acquires the reaction of the user 10.

Specifically, the behavior determination unit 236 determines, as a behavior of the robot 100, an utterance to the user 10 for proposing the content of the driving, and the behavior control unit 250 controls the control target 252 and makes an utterance to the user 10 for proposing the content of the driving. Here, the “content of the driving” includes, for example, content such as a case where it is desired to enjoy driving at a low speed while observing surrounding scenery, a case where it is desired to travel safely and quickly to a destination while complying with laws and regulations, and the like. In addition, the “content of the driving” may include driving at a predetermined distance or more from a preceding vehicle traveling ahead, driving using adaptive cruise control (ACC), and ecological driving while being conscious of fuel consumption. The user state recognition unit 230 recognizes the state of the user 10 based on the information analyzed by the sensor module unit 210, and the emotion determination unit 232 determines an emotion value indicating the emotion of the user 10 based on the information analyzed by the sensor module unit 210 and the state of the user 10 recognized by the user state recognition unit 230. The behavior determination unit 236 determines whether the reaction of the user 10 is positive, based on the state of the user 10 recognized by the user state recognition unit 230 and the emotion value indicating the emotion of the user 10, and determines, as the behavior of the robot 100, whether to execute processing of giving advice in accordance with the content of the driving proposed to the user 10, or to propose another content of the driving to the user 10.

(Step 5-1) In a case where the reaction of the user 10 is positive, the robot 100 executes processing of giving advice according to the proposed content of the driving.

Specifically, in a case where it is determined to execute processing of giving advice of the driving according to the content of driving proposed to the user 10 as the behavior of the robot 100, the behavior control unit 250 gives advice of the driving to the user 10.

(Step 5-2) In a case where the reaction of the user 10 is not positive, the robot 100 determines the content of another driving to be proposed to the user 10.

Specifically, in a case where it is determined to propose another driving advice to the user 10 as the behavior of the robot 100, the behavior determination unit 236 adds a fixed sentence such as “WHAT OTHER TYPES OF DRIVING DOES THE USER LIKE?” to the text representing the preference of the user 10 regarding the driving of the vehicle, the emotion of the user 10, the emotion of the robot 100, and the content stored in the history data 222, inputs the text to a sentence generation model, and acquires the recommended content regarding the driving of the vehicle. Then, the processing returns to Step 4 described above, and the processing of Steps 4 to 5-2 described above is repeated until it is determined to execute the processing of continuing the driving advice proposed to the user 10.

In this regard, the robot 100 can execute processing of giving driving advice according to the preference of the user, the situation of the user, and the reaction of the user.

With regard to the above embodiments, the following supplementary notes are further disclosed.

### Supplementary Note 1

A behavior control system including
a user state recognition unit that recognizes a user state including a behavior of a user,
an emotion determination unit that determines an emotion of the user or an emotion of a robot,
and a behavior determination unit that determines a behavior of the robot corresponding to the user state and the emotion of the user or a behavior of the robot corresponding to the user state and the emotion of the robot based on a sentence generation model having an interaction function of causing the user to interact with the robot, in which
the behavior determination unit gives advice for improving a driving skill by engaging in conversation with a driver driving a vehicle based on at least one of the preference of the driver, the driving situation, or the reaction.

### Supplementary Note 2

The behavior control system according to Supplementary Note 1, in which the behavior determination unit changes the form of advice based on the emotion of the user.

### Supplementary Note 3

The behavior control system according to Supplementary Note 1, in which the robot is mounted on a stuffed toy and provided in a vehicle interior of the vehicle.

### Supplementary Note 4

The behavior control system according to Supplementary Note 1, in which the robot is connected to a control target device mounted on the stuffed toy in a wireless or wired manner.

### Other Embodiment 17

As another embodiment, the robot 100 may be applied to a robot mounted on a device such as an electrical appliance, a computer, an automobile, a motorcycle, or a toy, or the robot 100 may control a device such as an electrical appliance, a computer, an automobile, a motorcycle, or a toy. Furthermore, the robot 100 may be mounted on a stuffed toy, or may be applied to a control device connected to a control target device (speaker or camera) mounted on the stuffed toy in a wireless or wired manner. Other embodiments are specifically configured as follows. For example, the robot 100 may be applied to control of an AI social gathering excitement machine.

The AI social gathering excitement machine controlled by the robot 100 functionally includes a topic providing unit that provides a topic for exciting a social gathering.

The robot 100 executes processing of providing a topic that facilitates interaction between the participants of the social gathering according to the preference of the participants of the social gathering as the user, the situation of the user, and the reaction of the user by Steps 1 to 5-2 below.

(Step 1) The robot 100 acquires the state of the user 10, the emotion value of the user 10, the emotion value of the robot 100, and the history data 222. Specifically, processing similar to above steps S100 to S103 is performed to acquire the state of the user 10, the emotion value of the user 10, the emotion value of the robot 100, and the history data 222.

(Step 2) The robot 100 acquires the preference of the user 10 regarding the topic of the social gathering. The topics include a game for exciting the social gathering, a topic of conversation for exciting the social gathering, and the like.

Specifically, the behavior determination unit 236 determines, as a behavior of the robot 100, an utterance to the user 10 to ask a question about a preference regarding the topic of the social gathering, and the behavior control unit 250 controls the control target 252 and makes an utterance to the user 10 to ask the question about the preference regarding the topic of the social gathering. The user state recognition unit 230 recognizes the preference of the user 10 regarding the topic of the social gathering based on the information (for example, the user's answer) analyzed by the sensor module unit 210.

(Step 3) The robot 100 determines a topic of the social gathering proposed to the user 10.

Specifically, the behavior determination unit 236 adds a fixed sentence such as “AT THIS TIME, WHAT IS THE TOPIC OF THE SOCIAL GATHERING RECOMMENDED TO THE USER?” to the text representing the preference of the user 10 regarding the topic of the social gathering, the emotion of the user 10, the emotion of the robot 100, and the content stored in the history data 222, inputs the text into the sentence generation model, and acquires the recommended content regarding the topic of the social gathering. At this time, it is possible to propose the topic of the social gathering suitable for the user 10 by taking into consideration not only the preference of the user 10 regarding the topic of the social gathering but also the emotion of the user 10 and the history data 222. Furthermore, by taking into consideration the emotion of the robot 100, it is possible to make the user 10 feel that the robot 100 has an emotion.

(Step 4) The robot 100 proposes the topic of the social gathering determined in Step 3 to the user 10 and acquires the reaction of the user 10.

Specifically, the behavior determination unit 236 determines, as a behavior of the robot 100, an utterance to the user 10 for proposing a topic of the social gathering, and the behavior control unit 250 controls the control target 252 and makes an utterance to the user 10 for proposing the topic of the social gathering. The user state recognition unit 230 recognizes the state of the user 10 based on the information analyzed by the sensor module unit 210, and the emotion determination unit 232 determines an emotion value indicating the emotion of the user 10 based on the information analyzed by the sensor module unit 210 and the state of the user 10 recognized by the user state recognition unit 230.

The behavior determination unit 236 determines whether the reaction of the user 10 is positive based on the state of the user 10 recognized by the user state recognition unit 230 and the emotion value indicating the emotion of the user 10, and determines whether to execute processing of providing a topic of the social gathering proposed to the user 10 or to propose another topic to the user 10 as the behavior of the robot 100.

(Step 5-1) In a case where the reaction of the user 10 is positive, the robot 100 executes processing of providing a proposed topic of the social gathering.

Specifically, in a case where it is determined that the processing of providing the proposed topic of the social gathering to the user 10 is executed as the behavior of the robot 100, the behavior control unit 250 controls at least one of the display apparatus or the speaker, which is the control target 252, to execute the providing of the proposed topic of the social gathering proposed to the user 10.

(Step 5-2) In a case where the reaction of the user 10 is not positive, the robot 100 determines another topic to be proposed to the user 10.

Specifically, in a case where it is determined to propose another topic to the user 10 as the behavior of the robot 100, the behavior determination unit 236 adds a fixed sentence such as “ARE THERE ANY OTHER TOPICS OF THE SOCIAL GATHERING RECOMMENDED TO THE USER?” to the text representing the preference of the user 10 regarding the topic of the social gathering, the emotion of the user 10, the emotion of the robot 100, and the content stored in the history data 222, inputs the text to a sentence generation model, and acquires the recommended content regarding the topic of the social gathering. Then, the processing returns to Step 4 described above, and the processing of Steps 4 to 5-2 described above is repeated until it is determined to execute the processing of providing the proposed topic of the social gathering to the user 10.

In this regard, the robot 100 can execute processing of providing the topic of the social gathering according to the preference of the user, the situation of the user, and the reaction of the user.

With regard to the above embodiments, the following supplementary notes are further disclosed.

### Supplementary Note 1

A behavior control system including
a user state recognition unit that recognizes a user state including a behavior of a user,
an emotion determination unit that determines an emotion of the user or an emotion of a robot,
and a behavior determination unit that determines a behavior of the robot corresponding to the user state and the emotion of the user or the emotion of the robot based on a sentence generation model having an interaction function of causing the user to interact with the robot, in which
the behavior determination unit determines, as a behavior of the robot, providing a topic to a participant of a social gathering as the user, the topic facilitating interaction among the participants.

### Supplementary Note 2

The behavior control system according to Supplementary Note 1, in which the robot is mounted on a stuffed toy or is connected to a control target device mounted on the stuffed toy in a wireless or wired manner.

The disclosure of Japanese Patent Application No. 2023-67475 filed on Apr. 17, 2023, the disclosure of Japanese Patent Application No. 2023-67476 filed on Apr. 17, 2023, the disclosure of Japanese Patent Application No. 2023-67886 filed on Apr. 18, 2023, the disclosure of Japanese Patent Application No. 2023-67888 filed on Apr. 18, 2023, the disclosure of Japanese Patent Application No. 2023-67890 filed on Apr. 18, 2023, the disclosure of Japanese Patent Application No. 2023-67892 filed on Apr. 18, 2023, the disclosure of Japanese Patent Application No. 2023-67980 filed on Apr. 18, 2023, the disclosure of Japanese Patent Application No. 2023-68148 filed on Apr. 18, 2023, the disclosure of Japanese Patent Application No. 2023-68804 filed on Apr. 19, 2023, the disclosure of Japanese Patent Application No. 2023-68805 filed on Apr. 19, 2023, the disclosure of Japanese Patent Application No. 2023-68823 filed on Apr. 19, 2023, the disclosure of Japanese Patent Application No. 2023-68855 filed on Apr. 19, 2023, the disclosure of Japanese Patent Application No. 2023-69490 filed on Apr. 20, 2023, the disclosure of Japanese Patent Application No. 2023-69494 filed on Apr. 20, 2023, the disclosure of Japanese Patent Application No. 2023-69617 filed on Apr. 20, 2023, the disclosure of Japanese Patent Application No. 2023-69668 filed on Apr. 20, 2023, and the disclosure of Japanese Patent Application No. 2023-69669 filed on Apr. 20, 2023 are incorporated herein by reference in their entirety.

### REFERENCE SIGNS LIST

5 System
10, 11, 12 User
20 Communication network
100, 101, 102 Robot
200 Sensor unit
201 Microphone
202 Depth sensor
203 Camera
204 Distance sensor
210 Sensor module unit
211 Voice emotion recognition unit
212 Utterance understanding unit
213 Facial expression recognition unit
214 Face recognition unit
220 Storage unit
221 Reaction rule
222 History data
230 User state recognition unit
232 Emotion determination unit
234 Behavior recognition unit
236 Behavior determination unit
238 Memory control unit
250 Behavior control unit
252 Control target
280 Communication processing unit
300 Server
1200 Computer
1210 Host controller
1212 CPU
1214 RAM
1216 Graphic controller
1218 Display device
1220 Input/output controller
1222 Communication interface
1224 Storage device
1226 DVD drive
1227 DVD-ROM
1230 ROM
1240 Input/output chip

1. A behavior control system comprising:
a user state recognition unit that recognizes a state of a user including a behavior of the user;
an emotion determination unit that determines at least one of an emotion of the user or an emotion of a robot;
and a behavior determination unit that determines a behavior of the robot corresponding to the state of the user and the emotion of the user or a behavior of the robot corresponding to the state of the user and the emotion of the robot based on an interaction function of causing the user to interact with the robot, wherein
the robot is mounted on a conversational scheduler, and
the behavior determination unit sets a behavior plan of the user based on an interaction between the user and the robot.

2. The behavior control system according to claim 1, wherein the behavior control system notifies the user of the behavior plan of the user set by the behavior determination unit.

3. The behavior control system according to claim 2, wherein a notification mode when notifying the user of the behavior plan is changed according to a state of the user and an emotion of the user.

4. The behavior control system according to claim 1, wherein the robot is mounted on a stuffed toy or is connected to a control target device mounted on the stuffed toy in a wireless or wired manner.

5. (canceled)

6. A behavior control system comprising:
a user state recognition unit that recognizes a user state including a behavior of a user;
an emotion determination unit that determines an emotion of the user or an emotion of a robot;
and a behavior determination unit that determines a behavior of the robot corresponding to the user state and the emotion of the user or the emotion of the robot based on a sentence generation model having an interaction function of causing the user to interact with the robot, wherein
the behavior determination unit determines a walking method to be proposed to the user based on an interaction result with the user by the sentence generation model.

7. A behavior control system comprising:
a user state recognition unit that recognizes a user state including a behavior of a user;
an emotion determination unit that determines an emotion of the user or an emotion of a robot;
and a behavior determination unit that determines a behavior of the robot corresponding to the user state and the emotion of the user or the emotion of the robot based on a sentence generation model having an interaction function of causing the user to interact with the robot,
wherein the behavior determination unit presents the user with a condition or handling of clothes based on an interaction between the user and the robot.

8-20. (canceled)
